(19) United States
(12) Patent Application Publication
(10) Pub. No.: US 2026/0286332 A1
(43) Pub. Date: Sep. 24, 2026

(54) SITE-SPECIFIC ORTHOGONAL BIOCONJUGATE MODALITY AND APPLICATION THEREOF

(71) Applicant: GENEQUANTUM HEALTHCARE (SUZHOU) CO., LTD., Suzhou (CN)

(72) Inventors: Gang QIN, Suzhou (CN); Meijun XIONG, Suzhou (CN); Cao LV, Suzhou (CN); Lili SHI, Suzhou (CN)

(21) Appl. No.: 19/471,068

(22) PCT Filed: Mar. 29, 2024
(86) PCT No.: PCT/CN2024/084752

§ 371 Date: Sep. 30, 2025

(30) Foreign Application Priority Data
Mar. 30, 2023 (WO) PCT/CN2023/085098

(51) Int. Cl. C12N 9/64; A61K 47/68; C12N 9/24; C12N 9/96

(52) U.S. Cl. CPC C12N 9/6472; A61K 47/68033; A61K 47/6851; A61K 47/6879; C12N 9/2402; C12N 9/96; C12Y 302/01096; C12Y 304/2207

(57) ABSTRACT

Provided is the process of producing an immunoligand/payload conjugate, in particularly, relates to a novel enzyme site-specific conjugation technology with dual enzyme orthogonal catalysis, comprising: conjugating a first payload to an immunoligand through the enzymatic catalysis of a first enzyme, conjugating a second payload to the immunoligand through enzymatic catalysis of a second enzyme, wherein the first enzyme is different from the second enzyme.

### TECHNICAL FIELD

The disclosure relates to a process of producing an immunoligand/payload conjugate, in particularly, relates to a novel enzymatic site-specific conjugation technology with dual-enzyme orthogonal catalysis, and highly homogeneous and mutually orthogonal drug-loaded antibody-drug conjugates prepared thereby, especially a dual-payload antibody drug conjugate (dpADC).

### BACKGROUND

Antibody-drug conjugates (ADC) are composed of three parts: antibody, linker and small-molecule drug with high potency. The small-molecule payload is linked to an antibody that can specifically binds to the antigen over-expressed on the tumor cell surface, the resulting ADC is then internalized by the tumor cell to release the cytotoxic payload in the tumor microenviroment (TME) or inside the tumor cells, the released payload then exerts an anti-tumor effect with a minimized toxicity to the normal tissues. Therefore, ADC is also known as a class of targeted chemotherapy drugs with controllable side effects. There are 14 ADC drugs approved worldwide by 2023, and over 100 in the clinical development stage, while most of these ADCs are based on a single type of payload. However, the tumor is highly heterogeneous and prone to drug resistance. In traditional tumor chemotherapy, the combination of two or more chemotherapeutic agents is used to overcome the drug resistance and improve the anti-tumor efficacy (Jain S, Jain R, Das M, Agrawal A K, Thanki K, Kushwah V. Combinatorial bio-conjugation of gemcitabine and curcumin enables dual drug delivery with synergistic anticancer efficacy and reduced toxicity. RSC Adv 2014, 4, 29193.). For example, the combination of docetaxel and gemcitabine was used in clinical trials, the synergy of varied mechanisms of action and non-overlapping tumor killing effects was validated by enhanced patient survival in both the treatment of drug-sensitive and tolerant tumors models (Hensley M L, Maki R, Venkatraman E, Geller G, Lovegren M, Aghajanian C, et al. Gemcitabine and docetaxel in patients with unresectable leiomyosarcoma: results of a phase II trial. J Clin Oncol 2002, 20, 2824). However, the combined use of chemotherapy drugs may also have increaed toxicity. To go a step further, the ADC-based combination therapy is receiving a growing interest and being investigated in preclinical models as well as clinical studies. Generally speaking the most appealing ADC partners are those that offer synergistic or additive effects on cancer cells or TME without any unacceptable toxicities, currently the partners of ADC such as chemotherapeutic agents, molecularly targeted agents and immunotherapy are the forerunners that start to show elevated antitumor potency in a syngistic manner with ADC. (Fuentes-Antras J, Genta S, Vijenthira A, Siu L. L, Antibody-drug conjugates: in search of partners of choice Trends in Cancer 2023). Based on these existing research accomplishments, we envision that an even attacting therapeuatic solution to treat immune-resistant cancer with a minimized off-target toxicities would be a dpADC that combine MoA varied agents in one modality, two drugs can reach the target at the same time and exert their effects in a highly synergistic manner.

Currently, there are several types of conjugation technologies for the construction of dual-payload antibody-drug conjugate (Weil T, Kuan S L, Xu L. Contemporary Approaches for Site-Selective Dual Functionalization of proteins. Angew. Chem.int. Ed. 2021, 60, 13757.): combination of two kinds of chemical conjugation methods, combination of chemical and enzymatic conjugation methods, and combination of two enzymatic conjugation methods. The currently available methods for constructing dpADC are mainly the first two ways, while dual-enzyme-catalyzed orthogonal site-specific conjugation has hardly been reported.

With regard to the combination of two chemical conjugation methods, for example, Pfizer reported a first example of dual payload conjugation of an antibody and Fab fragment immobilized on solid phase (by binding to protein-A/L agarose beads) in 2016. Using a reduction-reoxidation strategy to uncap an engineered unnatural cysteine in bead bound Fab fragment, the uncapped cysteine was then conjugated to a first maleimide-functionalized linker-payload, following another wash step the inter-chain disulfide bond of Fab was then reduced with TCEP followed by conjugation of a second maleimide-based linekr-payload to afford the dual-payload antibody fragment conjugate. They also demonstrated solid phase dual-payload conjugation based on enzymatic and chemical conjugation relay. As a proof of concept, transglutaminase mediated conjugation introduced a bicyclo[6.1.0] nonyne (BCN) group to the antibody, followed by BCN-azide click reaction to install the first linker-payload, and then the second linker-payload was connected via cystine-maleimide chemical conjugation. Both the two protocols require a multi-step (more than 6 steps) process, which could bring about huge problems in CMC manufacturing. (Puthenveetil S, Musto S, Loganzo F, Tumey LN, O'Donnell CJ, Graziani E, Development of solid-phase site-specific conjugation and its application towards generation of dual labeled antibody and Fab drug conjugates Bioconjugate Chem. 2016, 27, 1030.).

Seagen reported a strategy of constructing dpADC by constructing a trifunctional linker and chemical conjugation step by step in 2017. The researchers synthesized a trifunctional linker containing two orthogonally protected cysteine and one maleimide connector. The maleimide functional group of the linker was coupled with the cysteine sulfhydryl group of the antibody by Michael addition reaction, and then was subjected to two cycles of selective deprotection and Michael addition to connect the toxin. A total of five chemical reactions were carried out to obtain MMAE/MMAF dual-payload ADC. This ADC showed stronger antigen-positive tumor cell killing effect than single-kind-drug loaded ADC in the cell activity test, but it did not show synergistic effect in the mouse activity test in vivo, which may be due to the consistent action mechanism of the two drugs. (Levengood M R, Zhang X, Hunter J H, Emmerton K K, Miyamoto J B, Lewis T S, Senter P D, Orthogonal Cysteine Protection Enables Homogeneous Multi-Drug Antibody-Drug Conjugates, Angew. Chem. Int. Ed. 2017, 56, 733.).

With regard to the combination of chemical and enzyme site-specific conjugation methods, for example, in 2019, Williams et al. reported that based on the strategy of transpeptidase catalysis and perfluoroaryl nucleophilic substitution reaction, they successfully constructed a dual-payload antibody-fluorescein conjugate, which has the potential to develop dpADC.

In 2022, Sutro also reported site-specific dpADC conjugation method by designing a transglutaminase recognition motif (Qtag) at the C-terminus of the antibody light chain, which has the amino acid sequence GLLQGA (SEQ ID NO: 48), and then pairing said Qtag-LC with a heavy chain containing the unnatural amino acid pAMF, and then Qtag-LC undergoes enzyme-catalyzed targeted coupling to the cytotoxic-PEG-primary amine compound via transglutaminase, while the pAMF residue in HC is capable of coupling to the TLR agonist-PEG-DBCO by SPAAC for conjugation (CN 114206392 A).

As a summary, the reported methods of constructing dpADC are mainly based on either dual chemical conjugation relay or enzymatic and chemical conjugation relay, both require a multi-step process, which might be CMC complicated and time consuming, and chemical conjugation often leads to a heterogenous DAR distribution, furthermore they are also more liable to pre-mature release in systemic circulation due to the unstable linker design originated from the intact cysteine-maleimide ring structure. All these will cause problems in CMC manufacturing as well as safety concerns in clinical applications. To address this, a more facile, simple, efficient conjugation technology for the preparation of homogenous dpADC is highly needed.

### SUMMARY

In the first aspect, provided is an enzymatic conjugation process for preparing an immunoligand/payload conjugate, comprising the following steps:
conjugating a first payload to an immunoligand through the enzymatic catalysis of a first enzyme,
conjugating a second payload to the immunoligand through enzymatic catalysis of a second enzyme,
wherein the first enzyme is different from the second enzyme.

In some embodiment, the enzymatic catalysis step of the first enzyme and the enzymatic catalysis step of the second enzyme are performed simultaneously or sequentially. In an embodiment, the enzymatic conjugation process comprises conjugating simultaneously the first payload and the second payload molecule to the immunoligand through the enzymatic catalysis of the first and the second enzyme. In another embodiment, the enzymatic conjugation process comprises conjugating a first payload to an immunoligand through the enzymatic catalysis of a first enzyme to obtain a first conjugate; and conjugating a second payload to the first conjugate through the enzymatic catalysis of a second enzyme, thereby obtaining the immunoligand/payload conjugate.

In some embodiments, the first enzyme comprises a ligase and/or the second enzyme comprises an endoglycosidase. In some embodiments, the first enzyme comprises an endoglycosidase and/or the second enzyme comprises a ligase.

In an embodiment, the endoglycosidase is N-acetylglucosaminidase, which covalently connects a donor containing oxazoline oligosaccharide to an immunoligand containing GlcNAc motif, and the donor containing oxazoline oligosaccharide further contains a payload.

In another general aspect, provided is a ligase fusion protein and/or endoglycosidase fusion protein. In an embodiment, the ligase is covalently linked to the self-labelling protein tag to form a ligase fusion protein. In an embodiment, the endoglycosidase is covalently linked to the self-labelling protein tag to form an endoglycosidase fusion protein.

In the second aspect, provided are immobilized enzymes, wherein the first enzyme and the second enzyme are independently immobilized on a support.

In the third aspect, provided is an immunoligand/payload conjugate prepared by the process of the present disclosure.

In the fourth aspect, provided is use of the immunoligand/payload conjugate prepared by the process of the present disclosure for the manufacture of a medicament for treating a proliferative disease.

In the fifth aspect, provided is a method for treating a proliferative disease, comprising administering a therapeutically effective amount of the immunoligand/payload conjugate prepared by the present disclosure to a subject in need thereof.

In the sixth aspect, provided is a dual-drug ADC comprising an antibody having a first linker payload conjugated to the light chain C-terminal and a second linker-payload conjugated to the heavy chain Asn-297, wherein the payloads in the first linker payload and the second linker payload are different.

### BRIEF DESCRIPTION OF THE DRAWINGS

FIG. 1 shows the SDS-PAGE electrophoregram of the purified endoglycosidase fusion protein (Halo-Endo S2-His) of the present disclosure. In the figure, 1 indicates that the sample is from cell fluid, 2 indicates that the sample is from supernatant fluid, 3 indicates that the sample is from flow-through fluid (the composition of the balance buffer used is 50 mM Tris, 150 mM NaCl, 20 mM imidazole, pH 7.4), and 4 indicates that the sample is from washing solution (the composition of washing solution is 50 mM Tris, 150 mM NaCl, 80 mM imidazole, pH 7.4). 5 indicates that the sample comes from the eluent (the composition of the eluent is: 50 mM Tris, 150 mM NaCl, 500 mM imidazole, pH 7.4), 6 indicates that the sample comes from the eluent (the composition of the eluent is: 50 mM Tris, 150 mM NaCl, 500 mM imidazole, pH 7.4), and 7 indicates that the sample comes from magnetic beads; The molecular weight of endoglycosidase fusion protein (Halo-Endo S2-His) is about 130 kDa.

FIG. 2 shows the ring-open molecular structure of the linker 2-DM1 intermediate (n is an integer between 1 and 100, and x is a —OH or —NH₂ group; A and B are isomers).

FIG. 3 shows an SDS-PAGE electrophoregram of the supernatant of the immobilized endoglycosidase fusion protein (Halo-Endo S2-His) in different incubation times.

FIG. 4 shows the results of SDS-PAGE assay analysis of antibody-drug conjugate of ADC-1.

FIG. 5 shows the results of HIC-HPLC assay analysis of antibody-drug conjugate of ADC-1.

FIG. 6 shows the results of SEC-HPLC assay analysis of antibody-drug conjugate of ADC-1.

FIG. 7 shows the results of HIC-HPLC assay analysis of antibody-drug conjugate of ADC-2.

FIG. 8 shows the results of SEC-HPLC assay analysis and analysis of antibody-drug conjugate of ADC-2.

FIG. 9 shows the results of SDS-PAGE assay analysis of one-step method and two-step method of dpADC-1.

FIG. 10 shows the results of RP-HPLC assay analysis of ADC drug dpADC-1.

FIG. 11 shows the results of SEC-HPLC assay analysis of ADC drug dpADC-1.

FIG. 12 shows the results of analysis of ADC drug dpADC-2 by RP-HPLC.

FIG. 13 shows the results of analysis of ADC drug dpADC-2 by SEC-HPLC.

FIG. 14 shows the results of inhibitory effects of different drugs on tumor cell proliferation.

FIG. 15 shows the results of the bystander killing effect of ADC drug dpADC-1 on ErbB2/Her2 negative tumor cells.

FIG. 16 shows the results of the bystander killing effect of ADC drug dpADC-2 on the proliferation of tumor cells with different expression levels of ErbB2/Her2.

FIG. 17 shows the results of analysis of ADC drug dpADC-3 by RP-HPLC.

FIG. 18 shows the results of analysis of ADC drug dpADC-3 by SEC-HPLC.

FIG. 19 shows the effect of ADC drug dpADC-3 on the Osimertinib-resistant NSCLC PDX model mice with moderate expression levels of ErbB3/Her3 in vivo. FIG. 19A shows the inhibitory effect of dpADC-3 on the Osimertinib-resistant NSCLC PDX model mice with moderate expression levels of ErbB3/Her3. FIG. 19B shows the effect of dpADC-3 on the body weight of the Osimertinib-resistant NSCLC PDX model mice with moderate expression levels of ErbB3/Her3.

FIG. 20 shows the effect of ADC drug dpADC-3 on the Osimertinib-sensitive NSCLC PC-9 model mice with low expression levels of ErbB3/Her3 in vivo. FIG. 20A shows the inhibitory effect of dpADC-3 on the Osimertinib-sensitive NSCLC PC-9 model mice with low expression levels of ErbB3/Her3. FIG. 20B shows the effect of dpADC-3 on the body weight of the Osimertinib-sensitive NSCLC PC-9 model mice with low expression levels of ErbB3/Her3.

### DETAILED DESCRIPTION

### General Definitions

Unless defined otherwise, all technical and scientific terms used herein have the same meaning as is commonly understood by one of skill in the art. In addition, the terms and experimental procedures relating to protein and nucleic acid chemistry, molecular biology, cell and tissue culture, microbiology and immunology are those terms and common procedures widely used in the art. When a trade name is present herein, it refers to the corresponding commodity or the active ingredient thereof. All patents, published patents applications and publications cited herein are hereby incorporated by reference. Meanwhile, for better understanding of the present disclosure, definitions and explanations of relevant terms are provided below.

As used herein, the expression “at least one” or “one or more” refers to one, two, three, four, five, six, seven, eight, nine or more, one hundred, two hundred, three hundred, four hundred, five hundred, six hundred, seven hundred, eight hundred, nine hundred or more, etc. As used herein, “a” and “an” unless clearly indicated to the contrary, should be understood to mean “at least one”.

When a certain amount, concentration, or other value or parameter is set forth in the form of a range, a preferable range, or a preferable upper limit or a preferable lower limit, it should be understood that it is equivalent to specifically revealing any range formed by combining any upper limit or preferable value with any lower limit or preferable value, regardless of whether the said range is explicitly recited. Unless otherwise stated, the numerical ranges listed herein are intended to include the endpoints of the range and all integers and fractions (decimals) within the range. For example, the expression “i” is an integer of 2 to 20 means that i is any integer of 2 to 20, for example, i can be 2, 3, 4, 5, 6, 7, 8, 9, 10, 11, 12, 13, 14, 15, 16, 17, 18, 19 or 20. Other similar expressions should also be understood in a similar manner.

The terms “about” or “approximately”, when used in connection with a numerical variable, such as a concentration, an isoelectric point (pI), a pH, a temperature or a certain range, generally means that the value of the variable and all values of the variable are within experimental error (for example, within a 95% confidence interval for the mean) or within ±10% of a specified value, or a wider range.

The term “optional” or “optionally” means the event described subsequent thereto may, but not necessarily happen, and the description includes the cases wherein the said event or circumstance happens or does not happen.

The expression “comprising” or similar expressions “including”, “containing” and “having” are open-ended, and do not exclude additional unrecited elements, steps, or ingredients. The expression “consisting of” excludes any element, step, or ingredient not designated. The expression “consisting essentially of” means that the scope is limited to the designated elements, steps or ingredients, plus elements, steps or ingredients that are optionally present that do not substantially affect the essential and novel characteristics of the claimed subject matter. It should be understood that the expression “comprising” encompasses the expressions “consisting essentially of” and “consisting of”.

As used herein, the definition of “biomolecule” encompasses proteins, nucleic acids, lipids, carbohydrates, small nucleotides, amino acids and derivatives thereof.

As used herein, a “nucleic acid” or a “polynucleotide” refers to a polymer of at least two nucleotides or nucleotide derivatives joined together by phosphodiester bonds, including deoxyribonucleic acid (DNA) and ribonucleic acid (RNA).

As used herein, a “vector” is a vehicle used to transfer exogenous nucleic acids into a host cell, where the exogenous nucleic acids are amplified or expressed. As used herein, the definition of “vector” encompasses plasmids, linearized plasmids, viral vectors, cosmids, phage vectors, phagemids, artificial chromosomes (e.g., yeast artificial chromosomes and mammalian artificial chromosomes), etc. As used herein, a vector could be expressible and/or replicable inside a host cell, meaning that the vector is able to express RNA polynucleotides or polypeptides and/or to produce multiple copies of the vector in the host cell. To be “expressible” or “replicable”, a vector could comprise nucleic acid sequences or elements operably linked to a promoter. As used herein, “operably linked” with reference to nucleic acid sequences or elements means that these nucleic acid sequences are functionally related to each other. For example, a promoter can be operably linked to a nucleic acid sequence encoding a polypeptide, whereby the promoter regulates or mediates the transcription of the nucleic acid. Those skilled in the art could select and use appropriate vectors for a particular purpose.

As used herein, “peptide”, “polypeptide” or “protein” refers to two or more amino acids covalently joined. Unless otherwise specified, these terms are interchangeably.

As used herein, “sequence identity” has an art-recognized meaning and the percent of sequence identity between two polypeptides can be calculated by aligning the two sequences using publicly available algorithms, such as the Basic Local Alignment Search Tool (BLAST) and the Fast Adaptive Shrinkage/Thresholding Algorithm (FASTA) (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994). While there are a number of methods to measure identity between two polypeptides, the term “identity” is well known to skilled artisans (Carrillo, H & Lipman, D 7 SIAM J Applied Math 48:1073 (1988)).

As used herein, the term “variant” refers to a protein having substitutions, deletions, insertions of one or more residues when compared to a reference protein. The reference protein can be a naturally occurring protein that can be isolated from natural source (i.e., a wild-type protein) or an engineered protein. As used herein, the function or activity of a variant, such as a sortase A variant or a Halo tag variant, is substantially similar with or comparable to or higher than that of the reference sortase A or Halo tag, respectively. As used herein, the function or activity of a variant, such as an Endo S2 variant or a Halo tag variant, is substantially similar with or comparable to or higher than that of the reference Endo S2 or Halo tag, respectively.

As used herein, the term “endoglycosidase” refers to an enzyme that has activities: 1) catalyzes the hydrolysis of internal glycosidic bonds of oligosaccharide chains and polysaccharides, and can be used to cleave polysaccharides from glycoproteins; 2) transfer the oligosaccharide moiety to the acceptor. In some embodiments, the “endoglycosidase” of present disclosure can be used to hydrolyze the β-(1→4) glycosidic bond between two N-acetyl glucose at the N-glycosylation site of the Fc region of the antibody, and also can be used to transfer oxazoline oligosaccharide to GlcNAc-anitibody acceptor. “Glucoside transfer activity” refers to the catalytic activation of saccharides linked to different receptor molecules, such as proteins, nucleic acids, oligosaccharides, lipids and small molecules. In present disclosure, a donor with oxazoline oligosaccharide can be connected to GlcNAc in Fc region of an antibody.

As used herein, the term “Halo tag” refers to a halogenated alkane dehalogenase or a variant thereof, which removes the halogen from the halogenated alkyl substrate (e.g., a reagent comprising a halogenated alkyl moiety-(CH₂)₂₋₃₀—X, where X is a halogen such as F, Cl, Br, I, especially Cl or Br) and forms a covalent bond with the remainder of the substrate. Mutant haloalkane dehalogenases have been described in, e.g., WO2006/093529 and WO2008/054821, which are incorporated herein by reference. Mutant halogenated alkane dehalogenases that can be used in the present disclosure can include but are not limited to mutants of Xanthobacter dehalogenase (such as Xanthobacter autotrophicus dehalogenase (DhIA)) or Rhodococcus dehalogenase (such as Rhodococcus rhodococcus dehalogenase (DhaA)), such as containing one or more substituents at the catalytic triad residue, for example, replace His272 with Phe/Ala/Gly/Gln/Asn or replace Asp106 with Cys or other alternatives, as described in WO2008/054821. Provided that the mutant haloalnated alkane dehalogenase is able to form covalent bonds with haloalkyl substrates.

As used herein, “His tags” are polypeptides composed of histidine, such as His-His(His₂), His-His-His(His₃), His-His-His-His(His₄)(SEQ ID NO: 49), His-His-His-His-His(His₅) (SEQ ID NO: 50), His-His-His-His-His-His (His₆) (SEQ ID NO: 51), His-His-His-His-His-His-His-His-His-His (His₁₀) (SEQ ID NO: 52).

As used herein, the term “fusion protein” refers to the protein product that is co-expressed by two or more genes obtained through DNA recombination technology. For example, by deleting the termination codon of the coding gene of the first protein, and then connecting the second protein gene with the termination codon, the co-expression of the two genes can be achieved. The endolycosidase fusion protein may further contain one or more additional elements, such as additional peptides or tags, such as Halo tags and/or His tags. In some embodiments, the endoglycosidase fusion protein basically retains the desired characteristics. The ligase fusion protein can further contain one or more additional elements, such as additional peptides or tags. In some embodiments, the ligase fusion protein basically retains the desired characteristics.

In the specific context of the present disclosure, the position of amino acids in the protein is defined as follows: (i) starting from the N-terminal; and (ii) designating the first amino acid position at the N-terminal as 1. The amino acid (such as Ser) at a given position (such as site 34) can be expressed as Ser34. An amino acid (such as His) at a given amino acid position (such as site 272) is replaced by another amino acid (such as Phe), which can be expressed as His272Phe. As used herein, a “ligase” refers to an enzyme that can catalyze the covalent linkage of two or more molecules. A ligase can specifically catalyze the conjugation between a first moiety comprising the recognition motif of the ligase donor substrate and a second moiety comprising the recognition motif of the ligase acceptor substrate to produce a target conjugate.

As used herein, the term “transpeptidation reaction” refers to a chemical reaction in which one or more amino acids (such as a peptide) is transferred from one molecule to another molecule. A transpeptidase is an enzyme that is able to catalyze a transpeptidation reaction between a donor substrate and an acceptor substrate. In a simplified transpeptidation reaction catalyzed by a sortase, the sortase first cleaves the recognition motif of a ligase donor substrate (also referred to as a donor recognition motif, such as LPXTG (SEQ ID NO: 35), when SrtA is used) to form a substrate-enzyme intermediate through formation of a thioester bond; next, the recognition motif of a ligase acceptor substrate (also referred to as an acceptor recognition motif, such as GGG) nucleophilically attacks the thioester bond to release the enzyme and form a new peptide bond between the two substrates. A transpeptidation reaction often results in conjugation of two parties to form a conjugate.

As used herein, the term “resin” refers to organic polymers that have softening or melting range after being heated, have a tendency to flow under the action of external forces during softening, and are solid, semi-solid or liquid at normal temperature. “Halogenated resin” refers to a new compound formed by replacing at least one functional group in the resin with a halogenated group.

As used herein, the term “conjugation” refers to the covalent linkage of at least two parties (e.g., at least two molecules or at least two ends of the same molecule).

As used herein, a “conjugate” can be prepared from at least two parties (e.g., at least two molecules or at least two ends/side chains of the same molecule) through covalent linkage.

As used herein, a “bioconjugate” refers to a conjugate with at least one of the conjugated parties being a biomolecule. Examples of bioconjugates include therapeutic molecules conjugated to polymer, lipid, antibody, peptide, aptamer, or small molecular ligands, such as siRNA conjugates, peptide hormone conjugates, peptide-peptide conjugates, peptide-drug conjugates, antibody-drug conjugates and multispecific antibodies, or the like.

The term “targeting molecule” refers to a molecule that has an affinity for a particular target (e.g., receptor, cell surface protein, cytokine, etc.). A targeting molecule can deliver the payload to a specific site in vivo through targeted delivery. A targeting molecule can recognize one or more targets. The specific target sites are defined by the targets it recognizes. For example, a targeting molecule that targets a receptor can deliver a cytotoxin to a site containing a large number of the receptor. Examples of targeting molecules include, but are not limited to antibodies, antibody fragments, binding proteins for a given antigen, antibody mimics, scaffold proteins having affinity for a given target, ligands, and the like.

As used herein, the term “antibody-drug conjugate (ADC)” refers to a conjugate comprising an antibody or an antibody fragment conjugated to a payload covalently.

As used herein, the terms “activity”, “enzymatic activity” and “catalytic activity” of a ligase (such as a sortase) refer to the ability of the ligase to catalyze a conjugation reaction and can be used interchangeably. As used herein, the catalytic activity of a sortase in a conjugation reaction, for example, a conjugation reaction between an antibody and a payload, can be expressed as conjugation efficiency (conjugation efficiency=(molars of conjugated antibody: molars of total antibody)×100%) or DAR (Drug-to-Antibody Ratio, average drug to antibody ratio for a given preparation of antibody drug conjugate) distribution.

As used herein, the term “antibody (Ab)” is an immunoglobulin (Ig) molecule or a derivative thereof that specifically binds to an antigen through at least one antigen-binding site. A “conventional” or “full-length” antibody typically consists of four polypeptides: two heavy chains (HC) and two light chains (LC). As used herein, the definition of “antibody” encompasses conventional antibodies, recombinant antibodies, multispecific antibodies (e.g., bispecific antibodies), fully human antibodies, non-human antibodies, humanized antibodies, chimeric antibodies, intrabodies, diabodies, nanobodies (i.e., single-domain antibodies, VHH domains), and anti-idiotypic antibodies. Also contemplated are members of any immunoglobulin type (e.g., IgG, IgM, IgD, IgE, IgA and IgY), any class (e g., IgG1, IgG2, IgG3, IgG4, IgAQ1 and IgA2) or subclass (e.g., IgG2a and IgG2b), or any derivatives thereof.

Those skilled in the art will understand that the categories of heavy chains include gamma, mu, alpha, delta or epsilon (γ, μ, α, δ, ε), among which there are some subclasses (for example, γ1-γ4). The nature of the chain determines that the “types” of antibodies are IgG, IgM, IgA, IgG or IgE respectively. Immunoglobulin subclasses (isoforms), such as IgG1, IgG2, IgG3, IgG4, IgG5, etc., have been fully characterized and the functional specificity given by them is also known. All kinds of immunoglobulins are within the protection scope of the disclosure. In some embodiments, immunoglobulin molecule is IgG. IgG usually contains two identical light chain polypeptides with a molecular weight of about 23,000 Daltons and two identical heavy chain polypeptides with a molecular weight of about 53,000-70,000. The four chains are connected by disulfide bond in “Y” configuration, and the light chain starts from the “Y” mouth and continues to surround the heavy chain through the variable region.

Light chain can be divided into kappa (κ) or lambda (λ). Each heavy chain can bind to k or λ light chain. Generally speaking, when immunoglobulin is produced by hybridoma, B cell or genetically engineered host cell, its light chain and heavy chain are bound by covalent bond, and the “tail” part of the two heavy chains is bound by covalent disulfide bond or non-covalent bond. In the heavy chain, the amino acid sequence extends from the N-terminal of the Y configuration forked end to the C-terminal at the bottom of each chain. The immunoglobulin K light chain variable region is Vκ; the immunoglobulin λ light chain variable region is Vλ.

Both light and heavy chains are divided into regions of structural and functional homology. The terms “constant” and “variable” are used according to function. The variable regions of the light chain (VL) and heavy chain (VH) determine the antigen recognition and specificity. The constant regions of light and heavy chains give important biological properties, such as secretion, Fc receptor binding, complement binding, etc. Conventionally, the number of constant regions increases as they become farther away from the antigen-binding site or amino terminal of the antibody. The N-terminal part is the variable region and the C-terminal part is the constant region; the CH3 and CL domains contain the carboxyl ends of the heavy chain and the light chain, respectively.

In naturally occurring antibodies, the six “complementary decision regions” or “CDRs” present in each antigen-binding domain are short, non-contiguous sequences of antigen-specific binding amino acids that form antigen-binding domains, assuming that the antibody assumes its three-dimensional conformation in an aqueous environment. The remaining amino acids in the antigen-binding domain, known as the “framework” region, show small intermolecular variability. Most of the framework regions are β-folded conformations, and CDR forms a circular structure connected to them, or in some cases forms a part of the β-folded structure. Therefore, the framework region positions the CDR in the correct orientation by forming a scaffold and thus through interchain non-covalent interactions. The antigen-binding domain of a CDR with a specific location forms a surface complementary to the epitope on the antigen that facilitates non-covalent binding of the antibody to its antigenic epitope. For a given heavy or light chain variable region, amino acids containing CDRs and framework regions can be identified by known methods by those skilled in the art (Kabat, E. et al., U.S. Department of Health and Human Services, Sequences of Proteins of Immunological Interest, (1983) and Chothia and Lesk, J.Mol.Biol., 196:901-917 (1987)).

CDRs as defined by Kabat and Chothia include the overlap or subset of amino acid residues when compared to each other. Nevertheless, CDRs that refer to antibodies or their variants in any sense are within the scope of the present disclosure. The exact residue number containing a specific CDR will vary depending on the sequence and size of the CDR. Those skilled in the art can generally determine which specific residues the CDR contains based on the amino acid sequence of the variable region of the antibody. A numbering system for variable region sequences applicable to any antibody was also defined by Kabat et al. Those skilled in the art can apply the “Kabat number” system to any variable region sequence without relying on other experimental data other than the sequence itself. The “Kabat number” refers to the numbering system proposed by Kabat et al, U.S. Dept. of Health and Human Services in “Sequence of Proteins of Immunological Interest” (1983). Antibodies can also use the EU numbering system.

The “light chain constant region” consists of a portion of the amino acid sequence from the light chain of the antibody. Preferably, the light chain constant region (CL) comprises at least one of a constant κ structural domain or a constant λ structural domain. A “light chain-heavy chain pair” is a collection of light and heavy chains that can form a dimer through a disulfide bond between the CL domain of the light chain and the CH1 domain of the heavy chain.

The “Fc region” is the tail region of the antibody, which interacts with some proteins of the cell surface receptor and complement system. The property allows the antibody to activate the immune system. In IgG, IgA and IgD antibody isoforms, the Fc region consists of two identical protein fragments derived from the second and third constant regions of the two heavy chains of the antibody. In IgM and IgE antibody isoforms, the Fc region contains three heavy chain constant structural domains (CH2-4). The Fc region of IgG has a highly conserved N-glycosylation site. Glycosylation of Fc fragments is required for Fc receptor-mediated activity and different glycoforms have different effects on the pharmacological properties of therapeutic antibodies.

Antibodies can be prepared by using conventional recombinant DNA techniques. Cell lines for antibody production can be selected, constructed and cultured using techniques well known to those skilled in the art. These techniques are described in various laboratory manuals and major publications. In this regard, technical references suitable for use in the present disclosure, such as Current Protocols in Immunology, edited by Coligan et al, Green Publishing Associates and Wiley-Interscience, John Wiley and Sons, New York (1991), Recombinant DNA Technology for Production of Protein Therapeutics in Cultured Mammalian Cells, D. L. Hacker, F. M. Wurm, in Reference Module in Life Sciences, 2017 are described below. The entire contents, including supplements, are incorporated herein by reference.

In some embodiments, the DNA encoding the antibody can be synthesized according to the amino acid sequence of the antibody as described herein by conventional methods, placed in an expression vector, and then transfected into host cells, and the transfected host cells are cultured in medium to produce the monoclonal antibody. In some embodiments, the expression antibody vector includes at least one promoter element, an antibody coding sequence, a transcription termination signal, and a polyA tail. Other elements include enhancer, Kozak sequence and donor and acceptor sites for RNA splicing on both sides of insertion sequence. Efficient transcription can be obtained with pre- and post-SV40 promoters, long terminal repeat sequences from retroviruses such as RSV, HTLV1, HIVI and early promoters of cytomegaloviruses, and also by using promoters of some other cells such as actin promoters. Suitable expression vectors may include pIRES1neo, pRetro-Off, pRetro-On, PLXSN, or Plncx, pcDNA3.1(+/−), pcDNA/Zeo(+/−), pcDNA3.1/Hygro(+/−), PSVL, PMSG, pRSVcat, pSV2dhfr, pBC12MI and pCS2, etc. Frequently used mammalian cells include 293 cells, Cos1 cells, Cos7 cells, CV1 cells, mouse L cells and CHO cells.

In some embodiments, the inserted gene fragment needs to contain a screening marker. Common screening markers include dihydrofolate reductase, glutamine synthetase, neomycin resistance, thaumatin resistance and other screening genes to facilitate the screening isolation of successfully transfected cells. The constructed plasmids were transfected into the host cells without the above genes, and the transfected cells were cultured in selective medium to produce the desired protein.

As used herein, an “antibody fragment” of an antibody refers to any portion of an antibody comprising fewer amino acid residues than a full-length antibody, such as an antigen-binding fragment that contains at least a portion of the variable domains (e.g., one or more CDRs) of the antibody and specifically binds to the same cognate antigen as the full-length antibody, or an Fc fragment that contains heavy chain constant regions of the antibody and binds to Fc receptors on the cell surface. Antibody fragments can be obtained by various methods, such as chemical or enzymatic treatment, chemical synthesis or recombinant DNA technology. Examples of antibody fragments include, but are not limited to, Fv (the fragment variable region), scFv (single-chain Fv fragment), dsFv (disulfide-stabilized variable fragment), scdsFv (single-chain disulfide-stabilized variable fragment), diabody, Fd (the fragment difficult), Fab (the fragment antigen binding), scFab (single-chain Fab), Fab′, F(ab′)₂, Fc (the fragment crystallizable region) and any derivatives thereof.

As used herein, the term “payload” refers to a functional moiety which is comprised in a conjugate, for example, linked via a linker. Examples of payload include, but are not limited to, small molecule compounds (also referred to as small molecule drugs, e.g., inhibitors and toxins (such as cytotoxins)), radionuclides (e.g., ²²⁵Ac, ²¹¹At, ²¹²Bi, ²¹³Bi, ⁶⁷Ga, ¹²³I, ¹²⁴I, ¹²⁵I, ¹³¹I, ¹¹¹In, ¹⁷⁷Lu, ^{191m}Os, ^{195m}Pt, ¹⁸⁶Re, ¹⁸⁸Re, ¹¹⁹Sb, ¹⁵³Sm, ^{99m}Tc, ²²⁷Th and ⁹⁰Y), glycans, PEG moieties, nucleic acids and analogues (e.g., interfering RNAs), tracer molecules (e.g., fluorophores and fluorescent molecules), polypeptides (e.g., protein tags, bioactive peptides, enzymes, antibodies and antibody fragments, and protein toxins) and peptidomimetics. As used herein, payloads comprising a linker (such as a linker comprising a recognition motif of the ligase substrate) and a payload as described above are considered.

As used herein, the term “natural amino acid” refers to an amino acid that is a protein constituent amino acid, including the common twenty amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine), and the less common selenocysteine and pyrrolysine.

As used herein, the term “unnatural amino acid” refers to an amino acid that is not a protein constituent amino acid. In particular, the term refers to an amino acid that is not a natural amino acid as defined above.

As used herein, the term “peptidomimetic” refers to a compound that mimics the conformation and desirable features of a particular peptide.

As used herein, a “receptor” refers to a structure inside or on the surface of a cell that binds a specific substance and causes a specific effect in the cell. Receptors may include T-cell receptors, B-cell receptors, and receptors of signaling molecules, cell growth factors and cytokines as described herein.

As used herein, the term “cell growth factor” refers to any substance that is capable of stimulating cellular growth, healing, proliferation, survival and differentiation. Examples of growth factors may include, but are not limited to, epidermal growth factor (EGF), fibrablast growth factor (FGF), transforming growth factor (TGF), platelet-derived growth factor (PDGF), teratocarcinoma-derived growth factor (TDGF), insulin-like growth factor (IGF), nerve growth factor (NGF), vascular endothelial growth factor (VEGF) and erythropoietin (EPO).

As used herein, the term “cytokine” refers to any substance released by the cells of the immune system and having an effect on other cells. Examples of cytokines may include chemokines, lymphokines, colony-stimulating fectors (CSFs), monocyte chemoattractant proteins (MCPs), angiogenesis factors, interleukins, interferons, tumor necrosis factors (TNFs), growth factors, and other secreted and cell surface molecules that transmit signals to other cells. Cytokines include, but are not limited to, INFα, INFβ, INFγ, IL-1, IL-2, IL-4, IL-6, IL-8/CXCL8, IL-10, IL-12, IL-13, IL-15, IL-17, IL-18, IL-23, IP-10/CXCL10, eotaxin/CCL11, MCP-1/CCL2, MIP-1α/CCL4, RANTES/CCL5, TNFα, TNFβ and growth factors.

A small molecule compound refers to a molecule with a size comparable to that of an organic molecule commonly used in medicine. The term does not encompass biological macromolecules (e.g., proteins, etc.), but encompasses low molecular weight peptides or derivatives thereof such as dipeptides, tripeptides, tetrapeptides, pentapeptides and the like. Typically, the molecular weight of the small molecule compound can be, for example, about 100 to about 2000 Da, about 200 to about 1000 Da, about 200 to about 900 Da, about 200 to about 800 Da, about 200 to about 700 Da, about 200 to about 600 Da, about 200 to about 500 Da. As used herein, a small molecule compound may also be known as a drug.

Cytotoxin refers to a substance that inhibits or prevents the expression activity of a cell, cellular function, and/or causes destruction of cells. In some cases, the cytotoxins currently used in ADCs may be more toxic than commonly used chemotherapeutic drugs. Examples of cytotoxins include, but are not limited to, drugs that target the following targets: microtubule cytoskeleton, DNA, RNA, kinesin-mediated protein transport, regulation of apoptosis. The drug that targets microtubule cytoskeleton may be, for example, a microtubule-stabilizing agent or a tubulin polymerization inhibitor. Examples of microtubule-stabilizing agents include but are not limited to taxanes. Examples of tubulin polymerization inhibitors include but are not limited to maytansinoids, auristatins, vinblastines, colchicines, and dolastatins. The DNA-targeting drug can be, for example, a drug that directly disrupts the DNA structure or a topoisomerase inhibitor. Examples of drugs that directly disrupt DNA structure include but are not limited to DNA double strand breakers, DNA alkylating agents, DNA intercalators. The DNA double strand breakers can be, for example, an enediyne antibiotic, including but not limited to dynemicin, esperamicin, neocarzinostatin, uncialamycin, and the like. The DNA alkylating agent may be, for example, a DNA bis-alkylator (i.e., DNA-cross linker) or a DNA mono-alkylator. Examples of DNA alkylating agents include but are not limited to pyrrolo[2,1-c][1,4]benzodiazepine (PBD) dimer, 1-(chloromethyl)-2,3-dihydrogen-1H-benzo[e]indole (CBI) dimer, CBI-PBD heterodimer, dihydroindolobenzodiazepine (IGN) dimer, duocarmycin-like compound, and the like. Examples of topoisomerase inhibitors include but are not limited to camptothecins and anthracyclines. The RNA-targeting drug may be, for example, a drug that inhibits splicing, and examples thereof include but are not limited to pladienolide. Drugs that target kinesin-mediated protein transport can be, for example, mitotic kinesin inhibitors including, but not limited to, kinesin spindle protein (KSP) inhibitors. Cytotoxin also includes those inhibiting cell proliferation, for example, tyrosine kinase inhibitors (TKIs), which include but are not limited to Lapatinib, Neratinib, Pyrroltinib, Afatinib, Gefitinib, Erlotinib and Osimertinib, or the derivatives thereof. In some preferable embodiments, cytotoxin is Osimertinib. In some preferable embodiments, cycotoxin is a derivative of Osimertinib, for example, cycotoxin has the following structure:

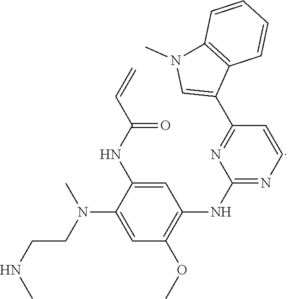 [SMILES: C=CC(=O)Nc1cc(Nc2nccc(-c3cn(C)c4ccccc34)n2)c(OC)cc1N(C)CCNC]

A spacer is a structure that is located between different structural modules and can spatially separate the structural modules. The definition of spacer is not limited by whether it has a certain function or whether it can be cleaved or degraded in vivo. Examples of spacers include but are not limited to amino acids and non-amino acid structures, wherein non-amino acid structures can be, but are not limited to, amino acid derivatives or analogues. “Spacer sequence” refers to an amino acid sequence serving as a spacer, and examples thereof include but are not limited to a single amino acid such as Leu, Gin, etc., a sequence containing a plurality of amino acids, for example, a sequence containing two amino acids such as GA, etc., or, for example, GGGS (SEQ ID NO: 36), GGGGSGGGGS (SEQ ID NO: 37), etc. Other examples of spacers include, for example, self-immolative spacers such as PAB (p-aminobenzyl), and the like.

The term “alkyl” refers to a straight or branched saturated aliphatic hydrocarbon group consisting of carbon atoms and hydrogen atoms, which is connected to the rest of the molecule through a single bond. The alkyl group may contain 1 to 20 carbon atoms, referring to C₁-C₂₀ alkyl group, for example, C₁-C₄ alkyl group, C₁-C₃ alkyl group, C₁-C₂ alkyl, C₃ alkyl, C₄ alkyl, C₃-C₆ alkyl. Non-limiting examples of alkyl groups include but are not limited to methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethyl butyl, 1,1-dimethylbutyl, 2,3-dimethylbutyl, 1,3-dimethylbutyl or 1,2-dimethylbutyl, or their isomers. A bivalent radical refers to a group obtained from the corresponding monovalent radical by removing one hydrogen atom from a carbon atom with free valence electron(s). A bivalent radical has two connecting sites which are connected to the rest of the molecule. For example, an “alkylene” or an “alkylidene” refers to a saturated divalent hydrocarbon group, either straight or branched. Examples of alkylene groups include but are not limited to methylene (—CH₂—), ethylene (—C₂H₄—), propylene (—C₃H₆—), butylene (—C₄H₈—), pentylene (—C₅H₁₀—), hexylene (—C₆H₁₂—), 1-methylethylene (—CH(CH₃)CH₂—), 2-methylethylene (—CH₂CH(CH₃)—), methylpropylene, ethylpropylene, and the like.

The term “cycloalkyl” refers to a cyclic saturated aliphatic group consisting of a carbon atom and a hydrogen atom, which is connected to the rest of the molecule by a single bond. Cycloalkyl can have 3-10 carbon atoms, i.e., “C₃-C₁₀ cycloalkyl”, such as cyclopropyl, cyclobutyl, cyclopentyl, cyclohexyl, cycloheptyl, cyclooctyl, cyclonononyl or cyclodecyl. The term “cycloalkylidene” refers to a divalent cycloalkyl group.

The term “heterocyclyl” means that one or more carbon atoms of the above-mentioned cycloalkyl are replaced by heteroatoms selected from nitrogen, oxygen and sulfur, such as aza, oxa or sulfur cyclopropyl, aza, oxa or sulfur cyclobutyl, pyrrolidinyl, pyrazolidinyl, imidazolidinyl, tetrahydrofuranyl, tetrahydrothiophenyl, piperidinyl, piperazinyl, tetrahydropyranyl, or tetrahydrothiopyranyl. The term “subheterocyclyl” refers to a divalent heterocyclyl group.

When referring to “substitution” herein, unless otherwise stated, the relevant substituent is selected from alkyl, halogen, amino, monoalkylamino, dialkylamino, nitro, cyano, formyl, alkylcarbonyl, carboxyl, alkylcarbonyloxy, aminocarbonyl, alkyloxycarbonyl, monoalkylaminocarbonyl, dialkylaminocarbonyl, formylamino alkylcarbonylamino, formyl(monoalkyl)amino, or alkylcarbonyl(monoalkyl)amino.

As used herein, when a group is combined with another group, the connection of the groups may be linear or branched, provided that a chemically stable structure is formed. The structure formed by such a combination can be connected to other moieties of the molecule via any suitable atom in the structure, preferably via a designated chemical bond. For example, when describing a combination of a C₁₋₄ alkylene with one of the groups including —CH₂—, —NH—, —(CO)—, —NH(CO)—, —(CO)NH—, the C₁₋₄ alkylene may form a linear connection with the above groups, such as C₁₋₄ alkylene-CH₂—, C₁₋₄ alkylene-NH—, C₁₋₄ alkylene-(CO)—, C₁₋₄ alkylene-NH(CO)—, C₁₋₄ alkylene-(CO) NH—, —CH₂-C₁₋₄ alkylene, —NH—C₁₋₄ alkylene, —(CO)—C₁₋₄ alkylene, —NH(CO)—C₁₋₄ alkylene, —(CO)NH—C₁₋₄ alkylene. The resulting bivalent structure can be further connected to other moieties of the molecule.

As used herein, the term “isoelectric point (pI)” is the pH (potential of hydrogen) value of an aqueous solution of a molecule (such as a protein) at which the molecule has no net surface charge and is expressed as pH units. The pI of a protein can be experimentally measured using methods well-known in the art, such as, imaged capillary isoelectric focusing (iCIEF) and capillary isoelectric focusing (CIEF). Different biomolecules (proteins, nucleic acids, polysaccharides, etc.) with different pIs may be differently charged at a given pH, allowing them to be separated by methods such as ion exchange chromatography or isoelectric focusing.

As used herein, a molecule having an “alkaline pI” means that the pI of the molecule is above 7.0. As used herein, a molecule having an “acidic pI” means that the pI of the molecule is below 7.0.

As used herein, a “protein tag” refers to a polypeptide that can be introduced to a molecule of interest to facilitate the detection, isolation, immobilization or capture of the molecule of interest, or improve one or more properties (such as expression level, solubility and stability) of the molecule of interest.

Ion exchange chromatography (IEX) separates biomolecules based on differences in their net surface charge and differences in their affinity towards the ion exchanger (also referred to as the medium, resin or stationary phase). It is a commonly used technique for biomolecule purification. For example, in the anion exchange chromatography, a protein with a pI below the buffer pH will have negative net surface charge and bind to the positively charged anion exchanger; however, another protein with a pI above the buffer pH will have positive net surface charge and do not bind to the positively charged anion exchanger, and will thereby pass through the medium along with the buffer.

As used herein, the term “support” refers to a water-insoluble substance that can be isolated from a reaction mixture in solid or semi-solid form, such as a surface, a gel, a polymer, a matrix, a particle, a resin, a bead or a membrane.

The term “ultrafiltration” or “UF” refers to membrane filtration technique which employs controlled pore, semi-permeable membranes to concentrate or fractionate dissolved molecules. Molecules much larger than the pores are retained in the feed solution and are concentrated in direct proportion to the volume of liquid that passes through the membrane. The pore size of the ultrafiltration membrane is generally between 1-100 nm.

The term “diafiltration” or “DF” refers to a technique that uses ultrafiltration membranes to completely remove, replace or lower the concentration of salts or solvents from solutions containing proteins, peptides, nucleic acids, and other biomolecules. The process selectively utilizes permeable (porous) membrane filters to separate the components of solutions and suspensions based on their molecular size. Ultrafiltration and diafiltration can be used in combination and referred to as UF/DF.

The following detailed description of the disclosure is intended to illustrate the non-limiting embodiments to enable those skilled in the art understand the technical solution, principle and practical application of the disclosure and those skilled in the art can modify and implement the disclosure in many forms.

### Dual-Enzyme Catalyzed Site-Specific Conjugation Process

The Present Disclosure Provides an Enzymatic Conjugation Process for Preparing an Immunoligand/Payload Conjugate, Comprising the Following Steps:
conjugating a first payload to an immunoligand through the enzymatic catalysis of a first enzyme,
conjugating a second payload to the immunoligand through enzymatic catalysis of a second enzyme,
wherein the first enzyme is different from the second enzyme.

In some embodiment, the enzymatic catalysis step of the first enzyme and the enzymatic catalysis step of the second enzyme are performed simultaneously or sequentially. In an embodiment, the enzymatic conjugation process comprises conjugating simultaneously the first payload and the second payload molecule to the immunoligand through the enzymatic catalysis of the first and the second enzyme. In another embodiment, the enzymatic conjugation process comprises conjugating a first payload to an immunoligand through the enzymatic catalysis of a first enzyme to obtain a first conjugate; conjugating a second payload to the first conjugate through the enzymatic catalysis of a second enzyme, thereby obtaining the immunoligand/payload conjugate.

In some embodiments, the first enzyme comprises a ligase and/or the second enzyme comprises an endoglycosidase. In some embodiments, the first enzyme comprises a ligase. In some embodiments, the second enzyme comprises an endoglycosidase. In some embodiments, the first enzyme comprises an endoglycosidase and/or the second enzyme comprises a ligase. In some embodiments, the first enzyme comprises an endoglycosidase. In some embodiments, the second enzyme comprises a ligase.

### Ligase

The ligase of the present disclosure can be any ligase of interest. Particularly, it can specifically catalyze the conjugation between a first moiety comprising the recognition motif of the ligase donor substrate and a second moiety comprising the recognition motif of the ligase acceptor substrate to produce a target conjugate.

In some embodiments, the ligase is a transpeptidase or a variant thereof. The transpeptidase can be naturally occurring or engineered. In some preferable embodiments, the ligase is a sortase, such as a sortase A (SrtA), sortase B (SrtB), sortase C (SrtC), sortase D (SrtD), sortase E (SrtE) or sortase F (SrtF), but not limited to. A “sortase” or “sortase enzyme” herein refers to an enzyme having sortase activity to catalyze a transpeptidation reaction, including for example, class A, class B, class C, class D, class E and class F sortases of the sortase enzyme superfamily (see, e.g., Dramsi, et al., Sorting sortases: a nomenclature proposal for the various sortases of Gram-positive bacteria, Research in Microbiology, (2005), 156:289-297; Bradshaw, et al., Molecular features of the sortase enzyme family, FEBS Journal, (2015), 282:2097-2114; Malik and Kim, A comprehensive in silico analysis of sortase superfamily, J Microbiol., (2019), 57 (6): 431-443; and EP3647419A1), but not limited thereto. Such an enzyme may be referred to as a SrtA, SrtB, SrtC, SrtD, SrtE or SrtF, but not limited thereto. A sortase can be naturally occurring or engineered. Naturally occurring sortase enzymes can be found in a variety of gram-positive bacteria, such as any strain, species or subspecies of the genera of Streptococcus (e.g., Streptococcus pneumoniae and Streptococcus pyogenes), Staphylococcus (e.g., Staphylococcus argenteus and Staphylococcus aureus), Bacillus (e.g., Bacillus anthracis) and Listeria (e.g., Listeria monocytogenes), but are not limited to. An engineered sortase, such as a sortase variant with substitutions, deletions or insertions of one or more amino acid residues, can be obtained from its natural counterpart via methods known in the art, such as protein engineering and chemical synthesis. Also contemplated are other variants (such as those with one or more active groups or labels) of any wild-type sortase known in the art. The provision is that the variant has identical or similar function of the wild-type sortase. Those skilled in the art will readily be able to identify a sortase and assign it to a specific class based on its sequence and other characteristics. However, the definition of sortase is not limited by any classification method or nomenclature system.

In some particular embodiments, the ligase is a sortase A (SrtA). A SrtA can be naturally occurring or engineered. Examples of SrtA include those described in, for example, U.S. Pat. No. 7,238,489 and Malik and Kim, 2019, such as those from any strain, subspecies or species of the genera of Streptococcus (e.g., Streptococcus pnetmoniae and Streptococcus pyogenes) Staphylococcus (e.g., Staphylococcus argenteus and Staphylococcus aureus), Streptomyces (e.g., Streptomyces coelicolor). Bacillus (e.g., Bacillus anthracis), Lactobacillus (e.g., Lactobacillus plantanim) and Listeria (e.g., Listeria monocytogenes), but are not limited to. The amino acid sequences of various SrtA can be found in, for example, U.S. Pat. No. 7,238,489 or public sequence databases (such as GenBank and Uniprot), the relevant content of which is incorporated herein by reference. Exemplary amino acid sequences of naturally occurring SrtA useful in the present disclosure can be proteins of Uniprot Accession Numbers: Q2FV99, A0A3S0JRJ4, A0A2T4Q430, A0A507SMZ3, A0A1F2JEX6, A0A364UNR7, A0A1J3ZU75, A0A0M2NSU2, A0A432A5V1, A0A1J4HB57, A0A4Q8MXV4, W1W5Z3, A0A2T4KDK7, A0A2K4DQX6, A0A2T4KHW3, A0A380FYB6, A0A2K4C0Y9, A0A4Q9WQB8, A0A121AFU6, A0A1Q8DH59, A0A5B2YTH7, A0A533IYI6, Q4L923, A0A1F1M8Z4, A0A2A1KC84 and A0A133Q671, but are not limited to. Engineered SrtA have been reported in various literatures, for example, WO 2016/014501, the relevant content of which is incorporated herein by reference. For example, an engineered SrtA having one or more substitutions (such as Pro94Arg, Aspl60Asn, Aspl65Ala, Lysl90Glu, Lysl96Thr, Glul05Lys and Glul08Gln) when compared to Q2FV99, or a truncated SrtA with an N-terminal 59 amino acids deletion compared to Q2FV99 as described in WO 2016/014501 may be considered. The amino acid sequence of a SrtA variant can have a sequence identity of at least about 85%, at least about 90%, at least about 95%, or at least about 99% with any other amino acid sequence described above. Also contemplated are variants (such as those with one or more active groups or labels) of any wild-type SrtA known in the art. The provision is that the variant has identical or similar function of the wild-type SrtA.

In some embodiments, the SrtA comprises an amino acid sequence selected from the group consisting of SEQ ID NOs: 1-26 (WT). In some other embodiments, the SrtA comprises an amino acid sequence selected from the group consisting of SEQ ID NOs: 1-26 and comprises amino acid substitutions at positions 34, 100, 105 and 136. In some embodiments, the amino acid residues at positions 34, 100, 105 and 136 are substituted with Ser, Asn, Ala and Thr (i.e., [Ser34][Asn100][Alal05][Thrl36], SNAT (SEQ ID NO: 53)), Tyr, Asn, Ala and Thr (i.e., [Tyr34][Asn100][Alal05][Thrl36], YNAT (SEQ ID NO: 54)), Trp, Asn, Asp and Thr (i.e., [Trp34][Asnl00][Aspl05][Thrl36], WNDT (SEQ ID NO: 55)), or Val, Asn, Asn and Ser (i.e., [Val34][Asnl100][Asnl05][Serl36], VNNS (SEQ ID NO: 56)), respectively. In a particular embodiment, the sortase A comprises the amino acid sequence of SEQ ID NO: 27, which is the SNAT counterpart of SEQ ID NO: 1.

In some embodiments, the sortase A comprises an amino acid sequence having a sequence identity of at least about 85%, at least about 90%, at least about 95%, or at least about 99% with an amino acid sequence selected from the group consisting of SEQ ID NOs: 1-26.

In some embodiments, the sortase A comprises an amino acid sequence having a sequence identity of at least about 85%, at least about 90%, at least about 95%, or at least about 99% with an amino acid sequence selected from the group consisting of SEQ ID NOs: 1-26 and comprises amino acid substitutions of SNAT, YNAT, WNDT or VNNS at positions 34, 100, 105 and 136.

In another aspect, provided is a SrtA comprising an amino acid sequence selected from the group consisting of SEQ ID NOs: 1-26 and comprising amino acid substitutions of SNAT, YNAT, WNDT or VNNS at positions 34, 100, 105 and 136 or an amino acid sequence having a sequence identity of at least about 85%, at least about 90%, at least about 95%, or at least about 99% thereto. In another particular aspect, provided is a SrtA comprising the amino acid sequence of SEQ ID NO: 27 or an amino acid sequence having a sequence identity of at least about 85%, at least about 90%, at least about 95%, or at least about 99% thereto.

In some embodiments, the sortase A comprises an amino acid sequence selected from any one of SEQ ID NO:1-27 or an amino acid sequence having at least about 85%, at least about 90%, at least about 95% or at least about 99% sequence identity therewith.

### Ligase Fusion Proteins

In a general aspect, provided is a ligase fusion protein comprising a ligase and self-labelling protein tags. In some embodiments, the ligase fusion protein comprising a ligase and a Halo tag. In some embodiments, the ligase fusion protein comprises a ligase and one or more self-labelling protein tags. In some embodiments, the ligase fusion protein comprises a ligase, a Halo tag and a His tag.

### Halo Tag

A Halo tag is a mutant haloalkane dehalogenase or a variant thereof that removes the halogen from a haloalkyl substrate (e.g., an agent comprising a haloalkyl moiety —(CH₂)₂₋₃₀—X, wherein X is a halogen like F, Cl, Br, I, particularly Cl or Br) and forms covalent linkage with the remaining moiety of the substrate. Mutant haloalkane dehalogenases have been described in, for example, WO 2006/093529 and WO 2008/054821, the relevant content of which is incorporated herein by reference. Mutant haloalkane dehalogenases useful in the present disclosure may include, but are not limited to, mutants of Xanthobacter dehalogenases (such as Xanthobacter antotrophiciis dehalogenase (DhIA)) or Rhodococcus dehalogenases (such as Rhodococcus rhodochrous dehalogenase (DhaA)), such as those comprising one or more substitutions at the catalytic triad residues, such as substitution of His272 with Phe/Ala/Gly/Gln/Asn or Asp 106 with Cys or other substitutions as described in WO 2008/054821. The provision is that the mutant haloalkane dehalogenase is able to form covalent linkage with a haloalkyl substrate.

In some preferable embodiments, the Halo tag comprises the amino acid sequence of SEQ ID NO: 28. In some embodiments, the Halo tag comprises an amino acid sequence having at least about 85%, at least about 90%, at least about 95%, or at least about 99% sequence identity with SEQ ID NO: 28.

### Embodiments Further Comprising Additional Elements and/or Modifications

Optionally, the ligase fusion protein may further comprise one or more additional elements, such as an additional polypeptide or a label. Preferably, the ligase fusion protein substantially preserves the desired properties. Those of skills in the art can select a suitable element based on the desired function or property of the fusion protein. Methods for introducing such elements are known in the art.

The additional polypeptide can be a protein tag with a desired property. Examples of protein tags may include, but be not limited to, reporter proteins, binding tags and solubility-enhancement tags. Examples of reporter proteins include, but are not limited to, fluorescent proteins (e.g., green fluorescent protein and its variants), AP (Alkaline Phosphatase) and HRP (Horseradish Peroxidase). A binding tag can effectively bind to a corresponding binding partner in a covalent or non-covalent manner. Examples of binding tags include, but are not limited to, polyhistidine tag (i.e., His tag, e.g., His₆ or His₈ tag), Fc tag (the constant region (domain 3 and 4) of immunoglobulin heavy-chain), calmodulin tag, maltose-binding protein (MBP), glutathione-S-transferase (GST), S tag (which interacts with ribonuclease S-protein), peptides that bind avidin/streptavidin/neutravidin (e.g., SBP tag, Strep tag and Strep tag II), Halo tag, SNAP tag and CLIP tag (engineered mutants of the DNA repair protein O⁶-alkylguanine-DNA allcyltransferase) and variants thereof. A solubility-enhancement tag, when expressed as a part of a recombinant protein, can typically enhance the expression level and solubility of the recombinant protein. Examples of solubility-enhancement tags include, but are not limited to, GB1 tag (the Bl domain of Streptococcal protein G), the Z domain of Staphylococcal protein A, SUMO (Small ubiquitin-related modifier), thioredoxin, GST and MBP. It is appreciated that the property of a protein tag does not constitute any limitation on the embodiments, and a protein tag can have one or more properties, for example, a reporter protein or a binding tag can be a solubility-enhancement tag as well.

The additional polypeptide can also be a short peptide that can serve as a linker, a spacer or an enzyme-cleavable sequence (such as a TEV protease recognition motif or a thrombin recognition motif). In some embodiments, a linker peptide (such as a polyglycine stretch, (G₄S)ₙ (SEQ ID NO: 57) wherein G is glycine, S is serine, and n is an integer of 1-6, preferably n is an integer of 2-5) which is rigid or flexible may be inserted between the ligase and the Halo tag to ensure the proper function of the fusion protein using methods known in the art. In some embodiments, the linker peptide is (G₄S)₂. In some embodiments, the ligase fusion protein comprises the amino acid sequence of SEQ ID NO: 29.

The ligase fusion protein may comprise one or more modifications, wherein the ligase, the Halo tag and the additional polypeptide (when applicable) are independently modified through, for example, substitution, deletion, addition, insertion of one or more amino acids, or introduction of moieties or active groups at one or more suitable residues, as long as the desired biological activities or functions of the modified fusion protein are substantially similar with that of the corresponding fusion protein.

### The pI of Ligase Fusion Protein

In some embodiments, the ligase has an isoelectric point (pI) of about 7.5 to about 10.0, the Halo tag has a pI of about 4.5 to about 5.0, and the pI of the ligase fusion protein is about 2.0 to about 4.5 pH units lower than that of the ligase. In those embodiments, comprising one or more additional elements (such as additional polypeptides or labels as defined above or a combination thereof) and/or modifications (such as amino acid substitutions, deletions, additions, insertions, or moieties or active groups), it is preferable that the desired pI difference between the ligase fusion protein and the ligase is achieved. In some embodiments, the additional polypeptide (if applicable) may have a specific pI that helps to achieve the desired pI of the ligase fusion protein.

In some embodiments, the pI of the ligase fusion protein is about 4.5 to about 6.5, such as about 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4 or 6.5. In some preferable embodiments, the pI of the ligase fusion protein is about 5.0 to about 6.0.

In some embodiments, the pI of the ligase is about 7.5 to about 8.5, such as 7.5, 7.6, 7.7, 7.8, 7.9, 8.0, 8.1, 8.2, 8.3, 8.4 or 8.5. In some embodiments, the pI of the ligase is about 8.6 to about 9.5, such as 8.6, 8.7, 8.8, 8.9, 9.0, 9.1, 9.2, 9.3, 9.4 or 9.5. In some embodiments, the pI of the ligase is about 9.6 to about 10.0, such as about 9.6, 9.7, 9.8, 9.9 or 10.0.

In some particular embodiments, the pI of the fusion protein is about 5.0 to about 6.0, and the pI of the ligase is about 7.6 to about 9.7.

In some embodiments, the ligase is a sortase. The sortase can be selected from SrtA, SrtB, SrtC, SrtD, SrtE and SrtF.

In some preferable embodiments, the SrtA comprises an amino acid sequence selected from the group consisting of SEQ ID Nos: 1-12 (WT). In some other embodiments, the SrtA comprises an amino acid sequence selected from the group consisting of SEQ ID Nos: 1-12 and comprises amino acid substitutions at positions 34, 100, 105 and 136. In some embodiments, the amino acid residues at positions 34, 100, 105 and 136 are substituted with Ser, Asn, Ala and Thr (SNAT), Tyr, Asn, Ala and Thr (YNAT), Trp, Asn, Asp and Thr (WNDT), or Val, Asn, Asn and Ser (VNNS), respectively. The pIs of these SrtA are listed in Table 1.

TABLE 1

| | WT | SNAT | YNAT | WNDT | VNNS |
|---|---|---|---|---|---|
| SEQ ID NO: 1 | 7.673 | 8.508 | 8.473 | 7.675 | 8.508 |
| SEQ ID NO: 2 | 8.867 | 9.114 | 9.066 | 8.874 | 9.114 |
| SEQ ID NO: 3 | 9.445 | 9.458 | 9.403 | 9.306 | 9.458 |
| SEQ ID NO: 4 | 8.874 | 9.123 | 9.075 | 8.881 | 9.123 |
| SEQ ID NO: 5 | 8.867 | 9.114 | 9.066 | 8.874 | 9.114 |
| SEQ ID NO: 6 | 9.114 | 9.306 | 9.255 | 9.123 | 9.306 |
| SEQ ID NO: 7 | 8.868 | 9.114 | 9.066 | 8.874 | 9.114 |
| SEQ ID NO: 8 | 8.867 | 9.114 | 9.066 | 8.874 | 9.114 |
| SEQ ID NO: 9 | 9.132 | 9.471 | 9.414 | 9.319 | 9.471 |
| SEQ ID NO: 10 | 8.896 | 9.33 | 9.275 | 9.141 | 9.33 |
| SEQ ID NO: 11 | 9.414 | 9.659 | 9.605 | 9.544 | 9.659 |
| SEQ ID NO: 12 | 8.896 | 9.33 | 9.275 | 9.141 | 9.33 |

In some embodiments, the sortase A comprises an amino acid sequence having a sequence identity of at least about 85%, at least about 90%, at least about 95%, or at least about 99% with an amino acid sequence selected from the group consisting of SEQ ID Nos: 1-12.

In some embodiments, the sortase A comprises an amino acid sequence having a sequence identity of at least about 85%, at least about 90%, at least about 95%, or at least about 99% with an amino acid sequence selected from the group consisting of SEQ ID NOs: 1-12 and comprises amino acid substitutions of SNAT, YNAT, WNDT or VNNS at positions 34, 100, 105 and 136.

In a particular embodiment, the sortase A comprises the amino acid sequence of SEQ ID NO: 27. It is the SNAT counterpart of SEQ ID NO: 1 and has a pI of 8.508.

In some embodiments, a linker peptide (such as a polyglycine stretch, (G₄S)ₙ wherein G is glycine, S is serine, and n is an integer of 1-6, preferably n is an integer of 2-5) which is rigid or flexible may be inserted between the ligase and the Halo tag to ensure the proper function of the fusion protein using methods known in the art. In some embodiments, the linker peptide is (G₄S)ₙ. In some embodiments, the ligase fusion protein comprises the amino acid sequence of SEQ ID NO: 29.

### Methods for Obtaining the Ligase Fusion Protein

The ligase, the Halo tag and the additional polypeptide (when applicable) can be fused in any manner. In some embodiments, the ligase is located at the N-terminal of the Halo tag. In some embodiments, the Halo tag is located at the N-terminal of the ligase. In some embodiments, a linker peptide (such as a polyglycine stretch, (G₄S)ₙ, wherein G is glycine, S is serine, and n is an integer of 1-6, preferably n is an integer of 2-5) which is rigid or flexible may be inserted between the ligase and the Halo tag to ensure the proper function of the fusion protein using methods known in the art. In some embodiments, the linker peptide is (G₄S)ₙ. In some embodiments, the ligase fusion protein comprises the amino acid sequence of SEQ ID NO: 29

The ligase fusion protein can be obtained using various techniques known in the art, such as expression from a nucleic acid obtained by recombinant DNA techniques, chemical synthesis, enzyme-catalyzed conjugation or chemical conjugation methods. In some preferable embodiments, the ligase fusion protein is a recombinant protein encoded by a nucleic acid comprising nucleic acid sequences encoding the ligase and the Halo tag. The recombinant protein can be expressed in and purified from a suitable host cell, such as a mammalian cell, a bacterium, a yeast cell or an insect cell, preferably a bacterium, such as E. coli.

### Nucleic Acids and Vectors

Also provided is a nucleic acid encoding the ligase fusion protein according to the present disclosure, comprising a first polynucleotide encoding the ligase and a second polynucleotide encoding the Halo tag according to the present disclosure, wherein the first and second polynucleotides are operably linked to a promoter. In some embodiments, the nucleic acid according to the present disclosure further comprises a third polynucleotide encoding an additional polypeptide operably linked to the ligase and the Halo tag. Examples of the third polypeptide are as described above.

In some embodiments, the first polynucleotide encodes a sortase A, and the second polynucleotide encodes a Halo tag. In some embodiments, the first polynucleotide encodes a sortase A having an amino acid sequence selected from the group consisting of SEQ ID NOs: 1-26, and the second polynucleotide encodes a Halo tag having the amino acid sequence of SEQ ID NO: 28. In a particular embodiment, the first polynucleotide encodes a SrtA having the amino acid sequence of SEQ ID NO: 27 and the second polynucleotide encodes a Halo tag having the amino acid sequence of SEQ TD NO: 28. In another particular embodiment, the nucleic acid encodes a ligase fusion protein having the amino acid sequence of SEQ ID NO: 29. It is appreciated by those of skill in the art that one or more nucleotides in the nucleic acid can be optimized without departing from the spirit of the present disclosure.

In some embodiments, the nucleic acid according to the present disclosure is prepared as recombinant nucleic acids, which may further comprise one or more additional polynucleotides, such as regulatory elements and polynucleotides encoding protein tags. Such regulatory elements may regulate the expression of the fusion protein according to the present disclosure, including, but not limited to, enhancers, insulators, internal ribosome entry sites (IRES). The recombinant nucleic acids comprising the nucleic acid according to the present disclosure can be prepared using molecular cloning techniques well known in the art, for example, chemical synthesis, site-specific mutagenesis and polymerase chain reaction (PCR) techniques (see Sambrook, J., E. F. Fritsch, and T. Maniatis (1989), Molecular cloning: a laboratory manual, 2nd ed., Cold Spring Harbor Laboratory, Cold Spring Harbor, N.Y).

In some embodiments, the nucleic acid according to the present disclosure is cloned into a vector, preferably, an expression vector expressible in a host cell (e.g., a bacterial, mammalian, yeast or insect cell). Those skilled in the art are able to select suitable expression vector based on the nature of the ligase fusion protein and the host cell to be used. In some embodiments, the vector is a bacterial expression vector expressible in bacteria, such as E. coli. In some embodiments, the expression vector can further contain one or more selective marker genes, for example, a neomycin or puromycin resistance gene. After expression, the fusion protein can be purified using methods known m the art according to the protein tag used. Depending on the type of host cells and purification strategies to be used, those of skills in the art are able to select suitable expression vectors, promoters, regulatory elements and protein tags.

### Immobilized Ligase

In another general aspect, provided is an immobilized ligase, comprising the ligase fusion protein according to the present disclosure immobilized to a support.

The support may be in solid form or semi-solid form made of any material. Non-limiting examples of the support may include, but are not limited to, a resin (e.g., an agarose resin, silicone resin, polymethyl methacrylate resin, epoxy resin or cellulose resin), gel (such as an alginate hydrogel), a bead/microsphere/particle (e.g., a polystyrene bead, a magnetic particle), a plate, a well, a tube, a film, a membrane, a matrix and glass (e.g., a glass slide).

In some preferable embodiments, the support is a resin. In some more preferable embodiments, the support is selected from the group consisting of agarose resin, silicone resin, polymethyl methacrylate resin and cellulose resin. In a particular embodiment, the support is a highly crosslinked agarose resin.

Methods of enzyme immobilization are known in the art, such as adsorption, covalent or non-covalent binding, entrapment, encapsulation, and cross linking. It is desirable that a maximum enzymatic activity of the ligase is preserved after immobilization and a minimum amount of free ligase is present in the conjugate product after the conjugation reaction. Preferably, the support is modified on the surface to comprise one or more functional groups such that the ligase fusion protein can be covalently immobilized on the support.

Preferably, the support comprises one or more chemically active functional groups that can form covalent bond with reactive groups (such as amines, thiols and carboxylates) of the ligase fusion protein or with reactive groups in a haloalkyl substrate, or the support comprises one or more binding partners of a corresponding binding tag/affinity label that is comprised in the ligase fusion protein. Correspondence relationship between chemically active functional groups and the reactive groups or correspondence relationship between binding tags/affinity labels and binding partners are well-known in the art. In some embodiments, the ligase is covalently immobilized on the support through a self-labelling protein tag, for example, a SNAP tag, a CLIP tag, a His tag, a Halo tag or a variant thereof. In an embodiment, the ligase is immobilized on the support by a covalent interaction between the haloalkyl linker and a Halo tag.

In some embodiments, the support comprises chemically active functional groups that can form covalent bond with reactive groups (such as amines, thiols and carboxylates) on the ligase fusion protein or with reactive groups in a haloalkyl substrate. In some particular embodiments, the support comprises functional groups selected from the group consisting of cyanate ester, isothiocyanates, isocyanates, carbodiimides, N-Hydroxysuccinimide (NHS) esters, amines, carbonates, epoxides, maleimides, haloacetyls, aziridines, ethyl chloroformate and aliphatic aldehydes.

In some embodiments, the support is an epoxy-activated resin, a CNBr (cyanogen bromide)-activated resin or an NHS-activated resin, preferably an epoxy-activated resin. In some particular embodiments, the support is an epoxy-activated agarose resin, preferably an epoxy-activated highly crosslinked agarose resin. In some preferable embodiments, before reacting with the haloalkyl substrate, the epoxy-activated resin is pre-processed to introduce amino group. In some preferable embodiments, the pre-processing of the epoxy-activated resin is performed using ammonia. In some preferable embodiments, the pre-processing of the epoxy-activated resin results in introduction of amino group on the oxirane ring and ring-opening of the oxirane ring gives a hydroxy group. Such hydroxy group are optionally end-capped in subsequent procedures of the preparation of the support. In a particular embodiment, the pre-processing of the epoxy-activated resin results in introduction of amino group on the oxirane ring and ring-opening of the oxirane ring gives a hydroxy group which is optionally esterified using esterification agent (e.g., acetylation agent like Ac₂O) in subsequent procedures of the preparation of the support. Such preprocessed epoxy-activated resin is within the scope of “epoxy-activated resin” as defined above. In some preferable embodiments, the resin is an agarose resin (such as a highly crosslinked agarose resin) or a polymethyl methacrylate resin.

In some other embodiments, the support comprises one or more binding partners of a corresponding binding tag/affinity label that is comprised in the ligase fusion protein, such as an additional tag or affinity label. Correspondence relationship between reactive groups or between binding tags/affinity labels and binding partners are well-known in the art. Examples of binding tags/affinity labels and the corresponding binding partners may include, but are not limited to, His tag and Ni²⁺, biotin/SPB tag/Strep tag/Strep tag II and streptavidin/avidin/neutravidin, GST tag and Glutathione, Fc tag and Protein A, calmodulin tag and Ca, MBP and amylose, S tag and ribonuclease S-protein, SNAP tag and benzylguanine (BG) derivatives, and CLIP tag and benzylcytosine (BC) derivatives.

In some preferable embodiments, the support is functionalized to form covalent interaction with the Halo tag by comprising haloalkyl linkers. The haloalkyl linkers can be introduced to the support by covalently connecting one or more functional groups comprised by the support to one or more reactive groups in haloalkyl substrates, and the support obtained thereby is also known as a haloalkyl linker-modified support. The haloalkyl linker-modified support is within the scope of “support” as defined above. Examples of haloalkyl substrates include but are not limited to those described in e.g., US20060024808A1 and WO2006093529. Haloalkyl substrates and methods for preparing such supports are described in, for example, U.S. Pat. Nos. 7,429,472, 7,888,086 and 8,202,700, Japanese Pat. No. 4748685, the relevant content of which is incorporated herein by reference.

The haloalkyl substrate may comprise a haloalkyl moiety comprising a primary or secondary halo group, preferably a primary halo group. The halo group in the haloalkyl moiety is selected from F, Cl, Br and I, preferably selected from Cl and Br. In some embodiments, the haloalkyl substrate has a structure of the following formula (I):

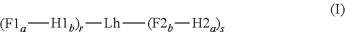

wherein,
F1 and F2 are independently a moiety comprising a reactive group which can form covalent bond with chemically active functional groups comprised by the support;
H1 and H2 are independently selected from halo C₂₋₃₀ alkyl;
Lh is a chemical bond or is a C₃₋₂₀₀ alkylene, and wherein one or more (—CH₂—) structures in the alkylene is optionally replaced by —O—, —NH—, —(CO)—, —NH(CO)— and —(CO)NH—;
Lh is optionally substituted with 1, 2 or 3 substituents selected from —O—C₁₋₁₀ alkyl, —NH—C₁₋₁₀ alkyl, —(CO)—C₁₋₁₀ alkyl, —NH(CO)—C₁₋₁₀ alkyl and —(CO)NH—C₁₋₁₀ alkyl;
a is 0 or 1, b is 0 or 1, provided that a and b are different;
r is an integer of 1 to 100;
s is an integer of 1 to 100.

In some embodiments, r is an integer of 1 to 10, for example, 1, 2, 3, 4, 5, 6, 7, 8, 9 or 10. In some embodiments, s is an integer of 1 to 10, for example, 1, 2, 3, 4, 5, 6, 7, 8, 9 or 10. In some embodiments, reactive group in F1 or F2 is selected from amino, amines, thiol group, thiols and active esters. In some embodiments, the active ester contains one or more carboxylic acid radicals (such as in carbonic acid monoester of suitable alcohol or phenol, e.g., electron-deficient phenol like 4-nitrophenol; or such as in NHS esters or sulfo-NHS esters) or one or more sulfonic acid radicals (such as in methane sulfonic acid active ester, e.g., MsO—).

In a particular embodiment, F1 or F2 is

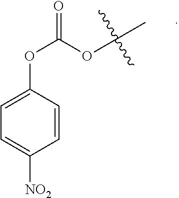 [SMILES: *OC(=O)Oc1ccc([N+](=O)[O-])cc1]

In some embodiments, H1 and H2 are independently selected from halo C₂₋₂₀ alkyl, preferably halo C₂₋₁₀ alkyl, especially halo C₆ alkyl. In some particular embodiments, the alkyl in H1 or H2 is a linear alkyl. In a particular embodiment. H1 or H2 is (CH₂)₂₋₃₀—X, preferably (CH₂)₂₋₂₀—X, more preferably (CH₂)₂₋₁₀—X, especially (CH₂)₆—X, wherein X is a halogen selected from F, Cl, Br and I.

In some preferable embodiments, the support is HaloLink™ resin (Promega).

In some more preferable embodiments, the support is a resin that may comprise a haloalkyl linker comprising the structure of —(CH₂)₂₋₃₀—X, wherein X is a halogen selected from F, Cl, Br and I. In a particular embodiment, the support is a haloalyl linker-modified resin, preferably an agarose resin or a polymethyl methacrylate resin, more preferably a highly crosslinked agarose resin.

In some particular embodiments, a is 1, b is 0, r is 1, s is 1, F1 is

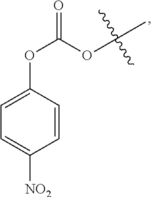 [SMILES: *OC(=O)Oc1ccc([N+](=O)[O-])cc1]

### Lh is

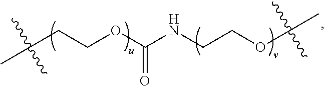

H2 is (CH₂)₂₋₂₀—Cl, and chloroalkyl substrate having the structure of formula (I-1):

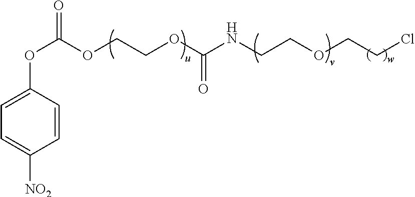

wherein, u is an integer of 1 to 20, v is an integer of 0 to 20, and w is an integer of 1 to 19.

In a particular embodiment, u is 3, v is 2, and w is 5, and the chloroalkyl substrate has the following structure of formula (I-1-1):

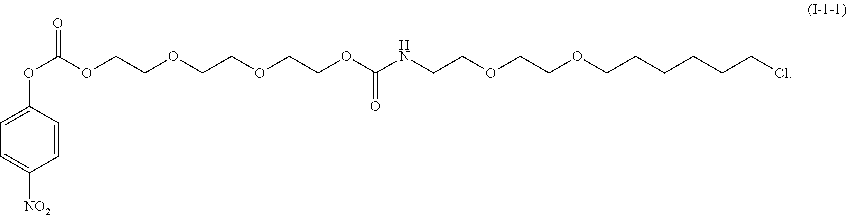 [SMILES: O=C(NCCOCCOCCCCCCCl)OCCOCCOCCOC(=O)Oc1ccc([N+](=O)[O-])cc1]

In a particular embodiment, the support is a chloroalkyl linker-modified support and has the structure of formula (II):

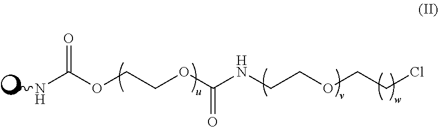

wherein u is an integer of 1 to 20, v is an integer of 0 to 20, and w is an integer of 1 to 19; [symbol] depicts the support and is a resin, a bead, a membrane, a gel, a matrix, a film, a plate, a well, a tube, a glass slide or a surface, preferably a resin, more preferably an agarose resin, a silicone resin, a polymethyl methacrylate resin or cellulose resin, and most preferably a highly crosslinked agarose resin. Note that for the sake of clarity, only a single chloroalkyl-linker moiety is depicted attached to the support, but it is understood that there would be many such chloroalkyl-linker moieties attached to the support.

In an embodiment, the chloroalkyl linker-modified support as shown in formula (II) is prepared using a resin, a bead, a membrane, a gel, a matrix, a film, a plate, a well, a tube, a glass slide or a surface as denoted by [symbol] , with the chloroalkyl substrate of formula (I-1).

In a particular embodiment, the chloroalkyl linker-modified support as shown in formula (II) is prepared from a preprocessed epoxy-activated resin: which is prepared by introduction of amino group on the oxirane ring of the epoxy-activated resin, and ring-opening of the oxirane ring during the preprocessing gives a hydroxy group which is optionally esterified using Ac₂O in subsequent procedures of the preparation of the support, and the support as shown in formula (II) has the structure of formula (II-1):

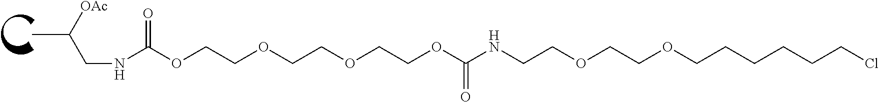

wherein,
the substructure

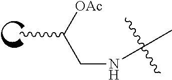 [SMILES: *NCC(C)OC(C)=O]

represents the preprocessed epoxy-activated resin, wherein the moiety

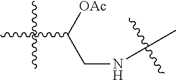

represents an oxirane ring which is reacted with amino group and ring-opened to give a hydroxy group esterified subsequently to form AcO—, and the moiety [symbol] represents the other part of the preprocessed epoxy-activated resin.

In some embodiments, the immobilized ligase has the following structure:
Support ---- Linker ---- HaloTag ---- Ligase
wherein
Support is a solid support, e.g., selected from resin, a bead, a membrane, a gel, a matrix, a film, a plate, a well, a tube, a glass slide or a surface, preferably a resin, more preferable an agarose resin, a silicone resin, a polymethyl methacrylate resin or cellulose resin, most preferably a highly crosslinked agarose resin;
Linker is a linker moiety, covalently bound to the Support, e.g., comprising a chain of 10 to 60 carbon atoms, optionally comprising one or more ether, ester, carbamate, and/or amide bonds, e.g., a linker moiety of Formula (II-1′) or (II′)

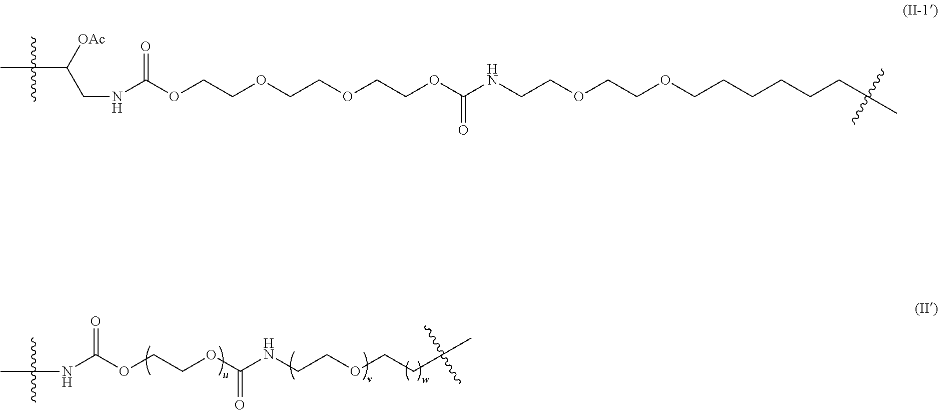

wherein u is an integer of 1-20, v is an integer of 0-20, and w is an integer of 1 to 19;
HaloTag is a Halo tag (haloalkane dehalogenase polypeptide), covalently bound to the linker;
Ligase is a ligase polypeptide;
wherein one or more “---- Linker ---- HaloTag ---- Ligase” moieties are bound to the same Support.

In some embodiments, an immobilized ligase comprising the linker moiety of Formula (II-1′) is obtained from the following reactions: 1) reaction of one or more chloroalkyl substrates with the support, forming a chloroalkyl linker-modified support; and 2) the subsequent reaction of the chloroalkyl linker-modified support with HaloTag (for example, the Halo tag comprised in the ligase fusion protein), obtaining the immobilized ligase.

### Endoglycosidase

The endoglycosidase of the present disclosure can be any endoglycosidase of interest. Particularly, the endoglycosidase is N-acetylglucosaminidase, which covalently connects a donor containing oxazoline oligosaccharide to an immunoligand containing GlcNAc motif, and the donor containing oxazoline oligosaccharide further contains a payload.

In some embodiments, the oxazoline oligosaccharide is one or more selected from the group consisting of: disaccharide oxazoline, trisaccharide oxazoline, tetrasaccharide oxazoline, pentasaccharide oxazoline, hexasaccharide oxazoline, heptasaccharide oxazoline, octasaccharide oxazoline, nonasaccharide oxazoline, decasaccharide oxazoline and undecasaccharide oxazoline.

In some embodiments, the oxazoline oligosaccharide has the following structure: the first hexose group or a derivative thereof-(the second hexose group or a derivative thereof)_{f}-β-D-glucopyranosyl oxazoline,
wherein f is 0, 1, 2, 3, 4, 5 or 6; β-D-glucopyranosyl oxazoline has the structure of:

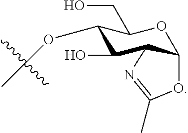

In some embodiments, the first hexose group or a derivative thereof in the oxazoline oligosaccharide is selected from glucosyl, mannosyl, galactosyl, or a derivative thereof; and/or the carbon at the 6-position of the first hexose group is in —C(O)— form.

In some embodiments, the second hexose group or a derivative thereof in the oxazoline oligosaccharide is independently selected from glucosyl, mannosyl, galactosyl or a derivative thereof in each occurrence.

In some embodiments, each monosaccharide portion of the oligosaccharide structure is linked by β-(1→4) glycosidic bonds.

In some embodiments, the first hexose group derivative and the second hexose group derivative in the oxazoline oligosaccharide are independently selected from a derivative in which a hydroxyl group of the uronic acid or monosaccharide is replaced by an acylamino group.

In some embodiments, the oxazoline oligosaccharide has the following structure:
the first hexose group or a derivative thereof-β-D-glucopyranosyl oxazoline, wherein the first hexose group or a derivative thereof is mannose or a derivative thereof.

In some embodiments, the oxazoline oligosaccharide has the following structure:
the first hexose group or a derivative thereof-β-D-glucopyranosyl oxazoline, wherein the first hexose group or a derivative thereof is a galactose or a derivative thereof.

In a preferable embodiment, the structure of the oxazoline oligosaccharide is as follows:

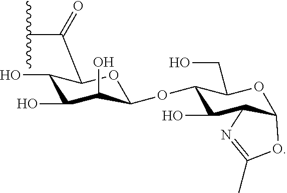

In some embodiments, the endoglycosidase is N-acetylglucosaminidase. In some embodiments, the N-acetylglucosaminidase is at least one selected from Endo S (Streptococcus pyogenes endoglycosidase-S), Endo F3 (Elizabethkingia miricola endoglycosidase-F3), Endo S2 (Endoglycosidase S2, Streptococcus pyogenes endoglycosidase-S2), Endo Sd (Endoglycosidase-Sd, Streptococcus pyogenes endoglycosidase-Sd) and Endo CC (Endoglycosidase-CC, Streptococcus pyogenes endoglycosidase-CC) or a variant thereof. In some embodiments, the N-acetylglucosaminidase is at least one selected from Endo H, Endo D, Endo F2, Endo F3, Endo M, Endo CC1, Endo CC2, Endo Om, Endo S and Endo S2 or a variant thereof. In some preferable embodiments, the N-acetylglucosaminidase is Endo S2 or a variant thereof.

### Endoglycosidase Fusion Protein

In another general aspect, provided is an endoglycosidase fusion protein comprising an endoglycosidase and a Halo tag. In some embodiments, the endoglycosidase fusion protein consists of a covalently linked endoglycosidase and a Halo. In some embodiments, the amino terminus of endoglycosidase is covalently connected with a Halo tag. In some embodiments, one end of endoglycosidase is covalently connected with a Halo tag, and the other end is covalently connected with a His tag. In some embodiments, the amino terminus of endoglycosidase is covalently connected with a Halo tag, and the carboxyl terminus is covalently connected with a His tag. In some embodiments, the amino terminus of endoglycosidase is covalently connected with a Halo tag, and the carboxyl terminus is covalently connected with a His tag, and the endoglycosidase is Endo S2. In an embodiment, the endoglycosidase fusion protein comprises an amino acid sequence of SEQ ID NO: 30, or an amino acid sequence having at least 90% identity therewith, or an amino acid sequence having one or more conservative amino acid substitutions compared to SEQ ID NO: 30. In some embodiments, the endoglycosidase fusion protein comprises or consists of an amino acid sequence as shown in SEQ ID NO: 30. In some embodiments, the endoglycosidase fusion protein comprises an amino acid sequence as shown in position 1 to position 1150th in SEQ ID NO: 30, or has at least 90% identity compared to the amino acid shown in position 1 to position 1150th in SEQ ID NO: 30, or has one or more conservative amino acid substitutions compared to the amino acid shown in position 1 to position 1150th in SEQ ID NO: 30. In some embodiments, the endoglycosidase fusion protein comprises or consists of the amino acid sequence shown in SEQ ID NO: 30 from position 1 to position 1150th.

### His Tag

In some embodiments, the His tag is a plurality of consecutive histidine residues. In some embodiments, the His tag is 3 histidine, 4 histidine, 5 histidine, 6 histidine, 7 histidine, 8 histidine, 9 histidine or 10 amino acids. In an embodiment, the His tag is His₆. In an embodiment, the His tag is His₈. In an embodiment, the His tag is His₁₀.

### The pI of Endoglycosidase Fusion Protein

In some embodiments, the pI of the endoglycosidase fusion protein is about 4-7. In some embodiments, the pI of the endoglycosidase fusion protein is about 4, 4.1, 4.3, 4.5, 4.7, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, or the interval value between any two values thereof (including the endpoint value).

### Methods for Obtaining the Endoglycosidase Fusion Protein

In one aspect, provided is a process for preparing the endoglycosidase fusion protein of the present disclosure, comprising: (a) providing a nucleic acid sequence of endoglycosidase; (b) one end of the nucleic acid sequence of the endoglycosidase is connected with the nucleic acid sequence of the Halo tag; the obtained nucleic acid sequence is cloned into a suitable vector, and then the vector is transformed into a suitable host cell, and the endoglycosidase fusion protein of the present disclosure is expressed in the host cell. In some embodiments, the method for preparing an endoglycosidase fusion protein of the present disclosure comprises: (a) providing a nucleic acid sequence of an endoglycosidase; (b) one end of the nucleic acid sequence of the endoglycosidase is connected with the nucleic acid sequence of the Halo tag; (c) the other end of the nucleic acid sequence of endoglycosidase is connected with the nucleic acid sequence of His tag.

### Immobilized Endoglycosidase

In one aspect, the present disclosure provides an immobilized endoglycosidase fusion protein comprising the endoglycosidase fusion protein of the present disclosure immobilized on a support.

In some embodiments, the endoglycosidase is covalently immobilized on the support through a self-labelling protein tag, for example, a SNAP tag, a CLIP tag, a His tag, a Halo tag or a variant thereof.

In some embodiments, the support comprises a haloalkyl linker, so that the endoglycosidase fusion protein is immobilized on the support through covalent interaction between the haloalkyl linker and the Halo tag.

In some embodiments, the support comprises a chloroalkyl linker such that the endoglycosidase fusion protein is immobilized on the support by covalent interaction between the chloroalkyl linker and the Halo tag. In some embodiments, the support that the endoglycosidase fusion protein immobilized on is the same as described in the above part of “Immobilized Ligase”.

In some embodiments, the immobilized endoglycosidase fusion protein has the following structure:
Support ---- Linker ---- Halo Tag ---- EndoS/EndoS2 ---- His Tag, or,
Support ---- Linker ---- Halo Tag ---- EndoS/EndoS2
wherein,
Support is a solid support, e.g., selected from resin, a bead, a membrane, a gel, a matrix, a film, a plate, a well, a tube, a glass slide or a surface. In an embodiment, the support is a resin. In an embodiment, the support is an agarose resin, a silicone resin, a polymethyl methacrylate resin or cellulose resin. In a particular embodiment, the support is a highly crosslinked agarose resin. In some embodiments, the support is selected from the group consisting of NHS activated resin, CNBr activated resin and epoxy activated resin;
Linker is a linker moiety, covalently bound to the Support, e.g., comprising a chain of 10 to 60 carbon atoms, optionally comprising one or more ether, ester, carbamate, and/or amide bonds, e.g., a linker moiety of Formula (II-1′) or (II′)

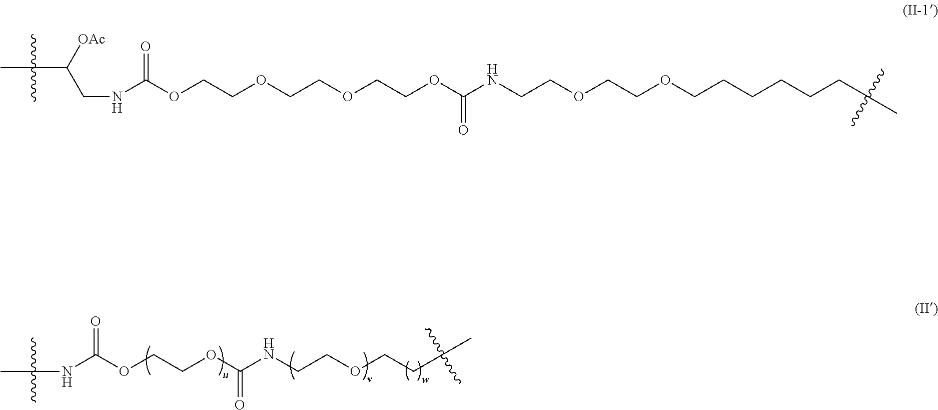

wherein u is an integer of 1-20, v is an integer of 0-20, and w is an integer of 1 to 19;
HaloTag is a Halo tag (haloalkane dehalogenase polypeptide), covalently bound to the linker;
EndoS/EndoS2 is a endoglycosidase;
wherein one or more “---- Linker ---- HaloTag ---- EndoS ---- HisTag” moieties are bound to the same Support.

In some embodiments, the immobilized endoglycosidase fusion protein has the following structure:
Support ---- Linker ---- Halo Tag ---- EndoS/EndoS2
wherein the Support, Linker, Halo Tag and EndoS/EndoS2 are as defined above;
wherein one or more “---- Linker ---- HaloTag ---- EndoS” moieties are bound to the same Support.

### Conjugation by Ligase

### Conjugation by Ligase Site-Specific Catalysis

In some embodiments, the conjugate has the structure of formula (III), the first moiety comprises T, and the second moiety comprises a linker-payload intermediate of formula (IV),

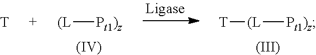

T comprises an immunoligand, which is optionally modified to have one of the recognition motif of the ligase donor substrate and the recognition motif of the ligase acceptor substrate;
L comprises a linker, which comprises the other of the recognition motif of the ligase donor substrate and the recognition motif of the ligase acceptor substrate;
P comprises a payload;
z is an integer of 1-20;
t1 is an integer of 1-20.
t1 denotes the number of payloads conjugated with a single linker to form the linker-payload intermediate of formula (IV). z denotes the number of formula (IV) compounds conjugated with a single T to form the compound of formula (III).

In an embodiment, z is selected from the following values: an integer of 1 to 10, 1 to 8, 1 to 6 or 1 to 4. In another embodiment, z is 1 or 2. In a very special embodiment, z is 2.

In some embodiments, the immunoligand is selected from an antibody, a modified antibody format, an antibody derivative or fragment and/or a mimetic antibody; preferably, the immunoligand contains an Fc terminus, which is optionally modified to have one of the recognition motif of the ligase donor substrate and the recognition motif of the ligase acceptor substrate. In another embodiment, T is a receptor, an antibody or an antibody fragment, which is optionally modified to have one of the recognition motif of the ligase donor substrate and the recognition motif of the ligase acceptor substrate. In a preferable embodiment, T is a molecule comprising an Fc fragment and an antigen-binding fragment of antibody, which is optionally modified to have one of the recognition motif of the ligase donor substrate and the recognition motif of the ligase acceptor substrate. In another embodiment, T is a soluble receptor, which is optionally modified to have one of the recognition motif of the ligase donor substrate and the recognition motif of the ligase acceptor substrate.

In some embodiments, T is a targeting molecule, which is optionally modified to have one of the recognition motif of the ligase donor substrate and the recognition motif of the ligase acceptor substrate. Targets recognized by the targeting molecules (such as antibodies or antigen-binding fragments thereof include but are not limited to CD19, CD22, CD25, CD30/TNFRSF8, CD33, CD37, CD44v6, CD56, CD70, CD71, CD74, CD79b, CD117/KIT, CD123, CD138, CD142, CD 174, CD227/MUC1, CD352, CLDN1&2, DLL3, ErbB2/HER2, ErbB3/HER3, GPNMB, ENPP3, Nectin-4, EGFRvTTT, SLC44A4/AGS-5, mesothelin, CEACAM5, PSMA, TIM1, LY6E, LIV1, Nectin4, SLITRK6, HGFR/cMet, SLAMF7/CS1, EGFR, BCMA, AXL, NaPi2B, GCC, STEAP1, MUC16, ETBR, EphA2, 5T4, FOLR1, LAMP1, Cadherin 6, FGFR2, FGFR3, CA6, CanAg, Integnn ccV, TDGF1, Ephnn A4, Trop2, PTK7, NOTCH3, C44A, FLT3.

In an embodiment, the targeting molecule is an anti-human HER2 antibody or antigen-binding fragment thereof, which is optionally modified to have one of the recognition motif of the ligase donor substrate and the recognition motif of the ligase acceptor substrate. Examples of anti-human HER2 antibodies include but are not limited to Pertuzumab and Trastuzumab.

In a preferable embodiment, the anti-human HER2 antibody is a recombinant antibody selected from the group consisting of monoclonal antibody, chimeric antibody, humanized antibody, antibody fragment, and antibody mimic. In an embodiment, the antibody mimic is selected from the group consisting of scFv, minibody, diabody, nanobody.

In an embodiment, the targeting molecule is an anti-human HER3 antibody or antigen-binding fragment thereof, which is optionally modified to have one of the recognition motif of the ligase donor substrate and the recognition motif of the ligase acceptor substrate. Examples of anti-human HER3 antibodies include but are not limited to Partritumab.

In a preferable embodiment, the anti-human HER3 antibody is a recombinant antibody selected from the group consisting of monoclonal antibody, chimeric antibody, humanized antibody, antibody fragment, and antibody mimic. In an embodiment, the antibody mimic is selected from the group consisting of scFv, minibody, diabody, nanobody.

For the conjugation with the compound of formula (IV), the targeting molecule of the present disclosure may comprise a modified moiety to connect with D1 or D2 in the compound of formula (V), namely the moiety in the linker which comprises a recognition motif of the ligase acceptor or donor substrate, cf. below. The introduction position of such modified moiety is not limited, for example, when the targeting molecule is an antibody, its introduction position can be, but not limited to, located at the C-terminal or the N-terminal of the heavy chain or light chain of the antibody.

In an alternative embodiment, a modified moiety for the conjugation with D1 or D2 in the compound of formula (V) can be introduced at a non-terminal position of the heavy chain or light chain of the antibody using, for example, chemical modification methods.

In an embodiment, the targeting molecule of the present disclosure is an antibody or antigen-binding fragment thereof, which may comprise terminal modification. A terminal modification refers to a modification at the C-terminal or N-terminal of the heavy chain or light chain of the antibody, which for example comprises a ligase recognition motif. In another embodiment, the terminal modification may further comprise spacer Sp2 comprising 2-100 amino acids, wherein the antibody, Sp2 and the ligase recognition motif are sequentially linked. In a preferable embodiment, Sp2 is a spacer sequence containing 2-20 amino acids. In a particular embodiment, Sp2 is a spacer sequence selected from the group consisting of GA, GGGS and GGGGSGGGGS, especially GA.

In a preferable embodiment, the light chain of the antibody or antigen-binding fragment thereof includes 3 types: wild-type (LC); the C-terminus modified light chain (LCCT), which is modified by direct introduction of a ligase recognition motif LPXTG, and C-terminus modified light chain (LCCT_{L}), which is modified by introduction of short peptide spacers plus the ligase donor substrate recognition motif LPXTG. The heavy chain of the antibody or antigen-binding fragment thereof includes 3 types: wild-type (HC); the C-terminus modified heavy chain (HCCT), which is modified by direct introduction of a ligase recognition motif LPXTG; and C-terminus modified heavy chain (HCCT_{L}), which is modified by introduction of short peptide spacers plus the ligase donor substrate recognition motif LPXTG. X can be any natural or non-natural single amino acid. When z in the compound of formula (IV) is 1 or 2, the combination of the above heavy and light chains can form 8 preferable antibody molecules, see the amino acid sequence table.

In a preferable embodiment, the light chain of the antibody or antigen-binding fragment thereof includes 3 types: wild-type (LC); the N-terminus modified light chain (LCNT), which is modified by direct introduction of a ligase recognition motif GGG, and N-terminus modified light chain (LCNT_{L}), which is modified by introduction of short peptide spacers plus the ligase acceptor substrate recognition motif GGG. The heavy chain of the antibody or antigen-binding fragment thereof includes 3 types: wild-type (HC); the N-terminus modified heavy chain (HCNT), which is modified by direct introduction of a ligase recognition motif GGG; and N-terminus modified heavy chain (HCNT_{L}), which is modified by introduction of short peptide spacers plus the ligase acceptor substrate recognition motif GGG.

The conjugates of the present disclosure can further comprise a payload. The payload is as described in the present disclosure.

### Linker of Ligase

In an embodiment, the linker, namely L in formula (III), is a compound of formula (V):

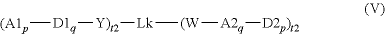

wherein,
D1 and D2 are independently a moiety comprising a recognition motif of the ligase acceptor or donor substrate;
A1 and A2 independently represents a bond connecting to the payload, or a moiety comprising a reactive group which can be conjugated with a payload;
Lk is a chemical bond, L₁-L₂-L₃ or L₁-L₂-L₃-L₄ or L₄-L₁-L₂-L₃ or L₄;
L₁ and L₃ are each independently selected from the group consisting of:
—CH₂—, —NH—, —(CO)—, —NH(CO)—, —(CO)NH—; and combination of a C₁₋₄ alkylene with one of the following groups: —CH₂—, —NH—, —(CO)—, —NH(CO)—, —(CO)NH—;
L₂ is absent or is a C₇₋₃₄ alkylene, and wherein one or more (—CH₂—) structures in the alkylene is optionally replaced by —O—;
L₁, L₂ and L₃ are each optionally and independently substituted with 1, 2 or 3 substituents selected from —OR₁ and —NR₁R₂;
R₁ and R₂ are each independently selected from the group consisting of hydrogen, —C₁₋₆ alkyl, —(CO)—C₁₋₆ alkyl and —S(═O)₂—C₁₋₆ alkyl;
L₄ is a peptide sequence (amide bond is formed by the condensation reaction of α-amino and carboxyl groups), wherein it contains an optionally derivatized Lys (Lysine) (number 1-100), or contains an optionally derivatized Cys (Cysteine) (number 1-100);
Y and W are each independently absent or selected from the group consisting of a cleavable sequence, spacer Sp1, and the combination thereof;
the cleavable sequence comprises an amino acid sequence which can be cleaved by enzyme, and the cleavable sequence comprises 1-10 ammo acids;
Sp1 is selected from the group consisting of a spacer sequence containing 1-20 amino acids, PAB, and the combination thereof;
p is 0 or 1, q is 0 or 1, provided that p and q are different;
t2 is as defined in formula (III).

In an embodiment, the linker of formula (V) is connected to the payload through A1 or A2, and is connected to the immunoligand T through the conjugation of D1 or D2 with the ligase acceptor substrate recognition motif comprised by the immunoligand T. Optionally, the ligase recognition motif in the immunoligand T exits in the form of a modified moiety, which is introduced to the immunoligand through, for example, recombinant methods or chemical modification methods.

In an embodiment, L₁, L₂ and L₃ are independently substituted with 1, 2, or 3 substituents seleted from —OR₁ and —NR₁R₂. Substitutions occur, for example, on (—CH₃), (—CH₂—) or

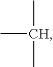

especially on (—CH₂—).

In an embodiment, L₂ is a C₇₋₃₄ alkylene, wherein the alkylene is a straight or a branched alkylene group, and optionally one or more of the (—CH₂—) structures in the alkylene can be replaced by —O—, and the alkylene is optionally substituted with 1; 2 or 3 substituents selected from —OR₁ and —NR₁R₂. In yet another embodiment, L₂ is selected from groups optionally substituted with 1, 2 or 3 substituents selected from —OR₁ and —NR₁R₂, wherein the said groups are as follows: methylene, ethylene, propylene, butylene, pentylene, hexylene, 1-methylethylene, 2-methylethylene, 2-methylpropylene and 2-ethylpropylene. In another embodiment, L₂ is —(C₂H₄—O)ᵢ—C₁₋₄ alkylene, i is an integer of 2 to 10. “—(C₂H₄—O)ᵢ—” represents a structure formed by polymerization of PEG units, wherein i indicates the number of PEG units. In another embodiment, L₂ is —(C₂H₄—O)ᵢ—C₁₋₂ alkylene. In a particular embodiment, L₂ is —(C₂H₄—O)ᵢ—C₂H₄—. In another embodiment, L₂ is —C₁₋₄alkylene-(O—C₂H₄)ᵢ—. In another embodiment, L₂ is —C₁₋₂alkylene-(O—C₂H₄)ᵢ—. In a particular embodiment, L₂ is —C₂H₄—(O—C₂H₄)ᵢ—. In an embodiment, i is selected from the following values: 2-10, 2-8, 2-6, 2-4 or 4-6. In a particular embodiment, i is 4.

In another embodiment of L₄ based on the desired number of conjugations, the ε-amino of lysine can either be used to introduce a maleimide functional group in A1 or A2 moieties by a suitable bifunctional crosslinking agent, or be used to form an amido bond with the α-carboxyl group of another lysine to form a branched chain, and then the α- and ε-aminos of the lysine in the branched chain can be used to introduce maleimide groups by a suitable bifunctional crosslinker. And so on, by increasing the number of the lysine in the main chain and/or branched side chain, the number of A1 or A2 moieties introduced by such a moiety La can achieve 1-1000.

In another embodiment of L₄, based on the desired number of conjugations, the mercapto group of each cysteine can be used to react with a maleimide functional group in A1 or A2. A1 or A2 can thus be connected to Lk. A1 and A2 each further comprises a reactive group which can be conjugated with a payload. By increasing the number of the cysteine in L₄, for example in the main chain and/or branched side chain of L₄, the number of A1 or A2 moieties introduced by such a moiety L₄ can achieve 1-1000.

In an embodiment, L₄ is optionally derivatized lysine.

In a preferable embodiment, the derivatization of lysine is selected from the group consisting of: 1) amidation of the carboxyl group, the resulting amide NH₂ being optionally substituted with a C₁₋₆ alkyl group; 2) linkage of the carboxyl group and/or the amino group to an amino acid fragment comprising 1-10 amino acids or a nucleotide fragment comprising 1-10 nucleotides, wherein the amino acid fragment is preferably Gly.

In an embodiment, Y and W are each independently absent or selected from the group consisting of a cleavable sequence, spacer Sp1, and the combination thereof. In a particular embodiment, Y is absent. In another particular embodiment, W is absent. In yet another particular embodiment, Y and W are both absent. In an embodiment, the cleavable sequence comprises an amino acid sequence that can be recognized as enzyme substrate and can be cleaved by the enzyme. In a particular embodiment, the cleavable sequence can be enzymatically cleaved in the lysosomal of the cell. In another particular embodiment, the cleavable sequence can be cleaved by protease, in particular by cathepsins. In yet another particular embodiment, the cleavable sequence can be cleaved by glutaminase. In an embodiment, the cleavable sequence is selected from the group consisting of a cathepsin restriction site, a glutaminase restriction site, and combinations thereof. In an embodiment, the cleavable sequence is selected from Phe-Lys, Val-Cit, Val-Lys, Gly-Phe-Leu-Gly (SEQ ID NO: 58), Ala-Leu-Ala-Leu (SEQ ID NO: 59), Val-Ala-PABC and the combination thereof.

In an embodiment, Y and W are each independently absent or selected from spacer Sp1. In another embodiment, Sp1 is a spacer sequence comprising 1-10, preferably 1-6, more preferably 1-4 amino acids. In a particular embodiment, Sp1 is Leu. In another particular embodiment, Sp1 is Gln. In an embodiment, Sp1 is PAB. In an embodiment, Sp is PABC. In yet another embodiment, Y and W are each independently selected from the group consisting of Phe-Lys-PAB, Val-Cit-PAB, Val-Ala-PABC and Val-Lys-PAB.

In an embodiment, the amino acids comprised by Y and/or W may be natural or unnatural. In a particular embodiment, Y is absent, or is amino acid fragment 1. Amino acid fragment 1 comprises 1-30 natural or unnatural amino acids, which are each independently the same or different. And amino acid fragment 1 is selected from the group consisting of: a cleavable sequence comprising 1-10 amino acids, a spacer sequence comprising 1-20 amino acids, and the combination thereof. In another particular embodiment, W is absent, or is amino acid fragment 2. Amino acid fragment 2 comprises 1-30 natural or unnatural amino acids, which are each independently the same or different. And amino acid fragment 2 is selected from the group consisting of: a cleavable sequence comprising 1-10 amino acids, a spacer sequence comprising 1-20 amino acids, and the combination thereof.

In an embodiment, p=0, q=1, the structure of the compound of formula (V) is as shown in the following formula (V-1):

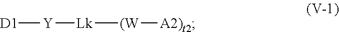

wherein, A2, D1, Y, Lk, and W are as defined in formula (V), respectively.

In another embodiment, p=1, q=0, the structure of the compound of formula (III) is as shown in the following formula (V-2):

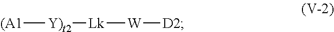

wherein, A1, D2, Y, Lk and W are as defined in formula (V), respectively.

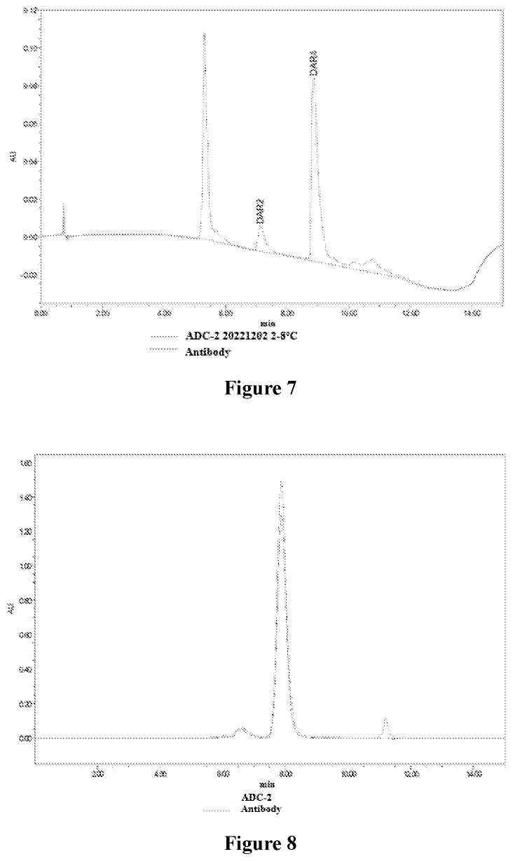

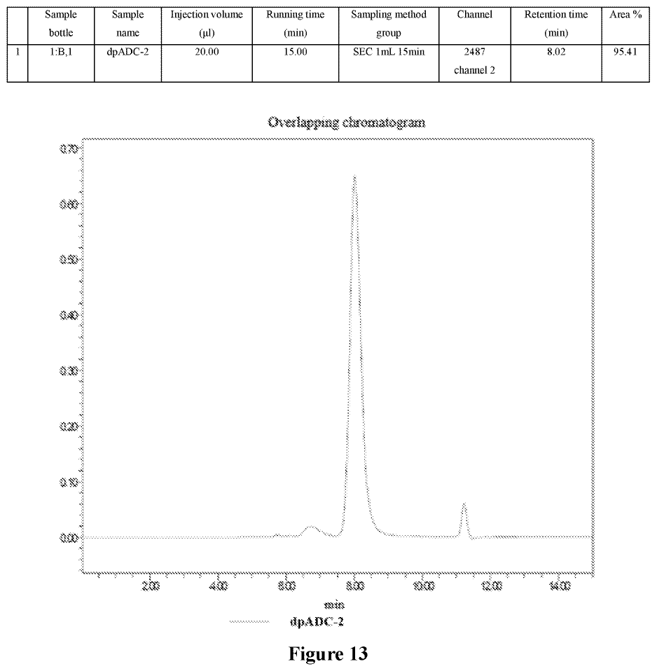

In an embodiment, suitable linker can be selected from those in any one of FIGS. 13 to 16 in WO 2014177042 A. In yet another embodiment, suitable linker can be selected from those in any one of FIGS. 7 to 10 in WO2015165413A.

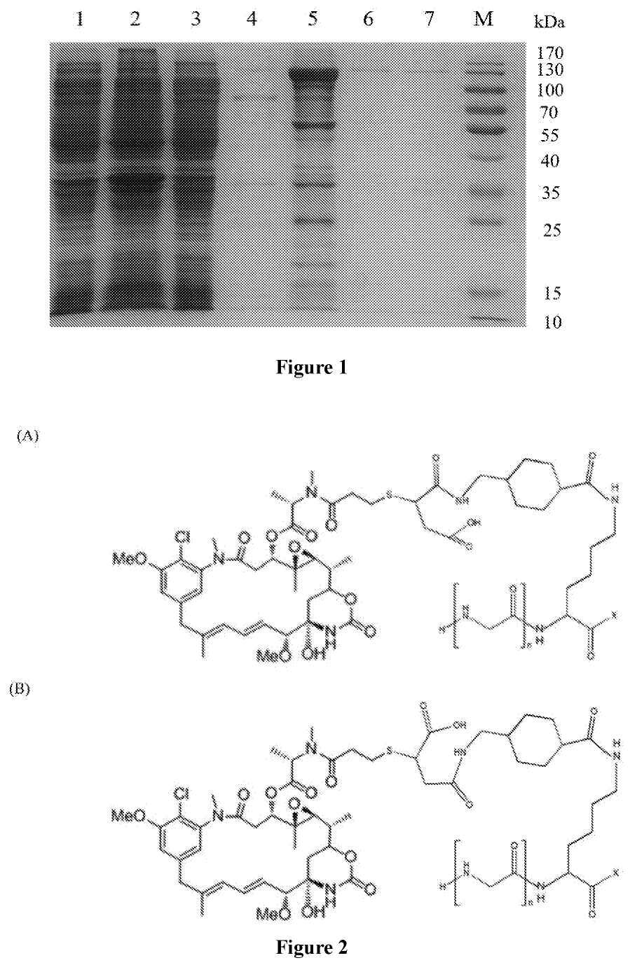

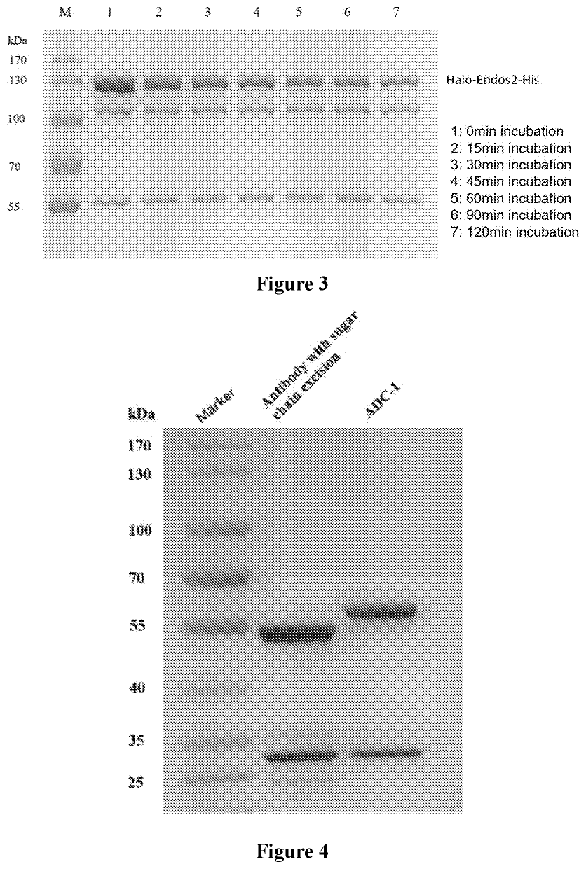

In an embodiment, suitable linker can be selected from those in any one of FIGS. 1 to 12 in WO 2014177042 A. In yet another embodiment, suitable linker can be selected from those in any one of FIGS. 3 to 6 in WO2015165413A.

In an embodiment, suitable linker can be selected from those in any one of Figures in page 16-29 of WO2022218331A1.

In an embodiment, suitable linker has the structure of formula (V′):

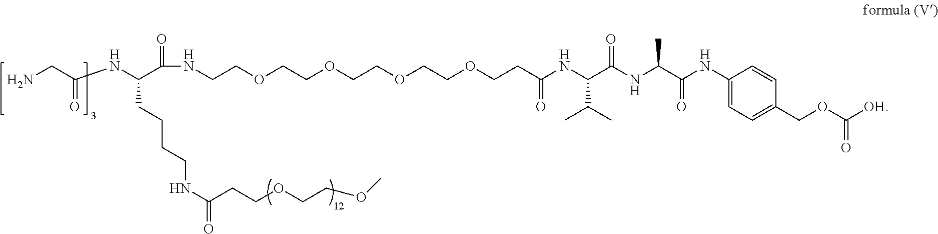

### Moiety Comprising Recognition Motif of the Ligase Acceptor or Donor Substrate

In an embodiment, the ligase is a transpeptidase. In an embodiment, the ligase is selected from the group consisting of a natural transpeptidase, an unnatural transpeptidase, variants thereof, and the combination thereof. Unnatural transpeptidase enzymes can be, but are not limited to, those obtained by engineering of natural transpeptidase.

In a preferable embodiment, the ligase is selected from the group consisting of a natural Sortase, an unnatural Sortase, and the combination thereof. The species of natural Sortase include SrtA, Srt B, SrtC, SrtD, SrtE, SrtF, etc. (see, e.g., US20110321183A1 and EP3647419A1). The type of ligase corresponds to the ligase recognition motif and is thereby used to achieve specific coupling between different molecules or structural fragments.

In an embodiment, the recognition motif of the ligase acceptor substrate is selected from the group consisting of oligomeric glycine, oligomeric alanine, and a mixture of oligomeric glycine/alanine having a degree of polymerization of 3-10. In a particular embodiment, the recognition motif of the ligase acceptor substrate is Gₙ, wherein G is glycine (Gly), and n is an integer of 3 to 10.

In some embodiments, the ligase is a SrtA, and the donor recognition motif can be LPXTG, wherein X is any natural or unnatural amino acid. In some embodiments, the ligase is a SrtB, and the donor recognition motif can be NPXTG (SEQ ID NO: 38), wherein X is any natural or unnatural amino acid. In some embodiments, the ligase is a SrtC, and the donor recognition motif can be LPXTG, wherein X is any natural or unnatural amino acid. In some other embodiments, the ligase is a SrtD, and the donor recognition motif can be LPXTA (SEQ ID NO: 39), wherein X is any natural or unnatural amino acid. In yet some other embodiments, the ligase is a SrtE, and the donor recognition motif can be LAXTG (SEQ ID NO: 40), wherein X is any natural or unnatural amino acid. In some other embodiments, the ligase is a SrtF, and the donor recognition motif can be LPXTG, wherein X is selected from the group consisting of A, R, N, D, Q, I, L and K.

In another particular embodiment, the ligase is a SrtA from Staphylococcus aureus. Accordingly, the ligase recognition motif may be the typical recognition motif LPXTG of the enzyme. In yet another particular embodiment, the recognition motif of the ligase donor substrate is LPXTGJ (SEQ ID NO: 41), and the recognition motif of the ligase acceptor substrate is Gₙ, wherein X can be any single amino acid that is natural or unnatural; J is absent, or is an amino acid fragment comprising 1-10 amino acids, optionally labeled. In an embodiment, J is absent. In yet another embodiment, J is an amino acid fragment comprising 1-10 amino acids, wherein each amino acid is independently any natural or unnatural amino acid. In another embodiment, J is Gₘ, wherein m is an integer of 1 to 10. In yet another particular embodiment, the recognition motif of the ligase donor substrate is LPETG (SEQ ID NO: 42). In another particular embodiment, the recognition motif of the ligase donor substrate is LPETGG (SEQ ID NO: 43). In an embodiment, the ligase is SrtB from Staphylococcus aureus and the corresponding donor substrate recognition motif can be NPQTN (SEQ ID NO: 44). In another embodiment, the ligase is SrtB from Bacillus anthracis and the corresponding donor substrate recognition motif can be NPKTG (SEQ ID NO: 45). In yet another embodiment, the ligase is SrtA from Streptococcus pyogenes and the corresponding donor substrate recognition motif can be LPXTGJ, wherein J is as defined above. In another embodiment, the ligase is SrtE from Streptomyces coelicolor, and the corresponding donor substrate recognition motif can be LAXTG. In yet another embodiment, the ligase is SrtA from Lactobacillus plantarum and the corresponding donor substrate recognition motif can be LPQTSEQ (SEQ ID NO: 46). The ligase recognition motif can also be other totally new recognition sequence for transpeptidase optimized by manual screening.

When coupling LPXTGJ with Gₙ, the upstream peptide bond of the glycine in the LPXTGJ sequence is cleaved by Sortase A, and the resulting intermediate is linked to the free N-terminal of Gₙ to generate a new peptide bond. The resulting amino acid sequence is LPXTGₙ (SEQ ID NO: 47). The sequences Gₙ and LPXTGJ are as defined above.

In some particular embodiments, the ligase is a SrtA from Staphylococcus aureus, the donor recognition motif is LPETGG, the acceptor recognition motif is GGG.

### Moiety Comprising Reactive Group

In an embodiment, A1 and A2 in formula (V) are each independently selected from the group consisting of amino compound, maleimide and derivative thereof, thiol compound, pyridyldithiol compound, haloacetic acid (haloacetylic acid), isocyanate. In another embodiment, the reactive groups in A1 and A2 are each independently selected from the group consisting of amino group, maleimide group, thiol group, pyridyldithio group, haloacetyl group, and isocyanate group.

In an embodiment, according to the structure of the reactive group therein, A1 and A2 can each independently covalently couple with a Michael acceptor (the acceptor molecule of Michael addition) through a disulfide bond, a thioether bond, a thioester bond, or a urethane bond. In a particular embodiment, A1 and A2 are each independently selected from optionally derivatized cysteines.

In another particular embodiment, A1 and A2 are each independently selected from optionally derivatized cysteines. In a preferable embodiment, the derivatization of cysteine is selected from the group consisting of: 1) amidation of the carboxyl group, the resulting amide NH₂ being optionally substituted with a C₁₋₆ alkyl group; 2) acylation of the amino group; and 3) linkage of the carboxyl group and/or the amino group to an amino acid fragment comprising 1-10 amino acids or a nucleotide fragment comprising 1-10 nucleotides, wherein the amino acid fragment is preferably Gly. In a particular embodiment, the derivatization of cysteine refers to amidation or linkage to glycine for the carboxyl group of cysteine.

In an embodiment, A2 is

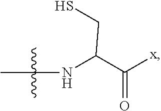

wherein x is selected from the group consisting of hydrogen, OH, NH₂, an amino acid fragment comprising 1-10 amino acids, and a nucleotide fragment comprising 1-10 nucleotides. In an embodiment, A1 is

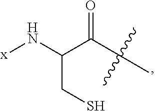

wherein x is selected from the group consisting of hydrogen, an amino acid fragment comprising 1-10 amino acids, and a nucleotide fragment comprising 1-10 nucleotides. In an embodiment, acylation of the amino group refers to the substitution with a C₁₋₆ alkylcarbonyl group for the amino group of cysteine.

In some embodiments of the linking unit of formula (V-1), wherein t2 is 1, D1 is Gₙ, G is glycine, A2 is

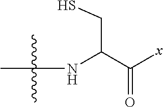

and the structure of the compound of formula (V-1) is as shown in the following formula (V-1-1):

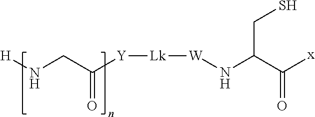

wherein n is an integer of 3 to 10;
x is selected from the group consisting of hydrogen, OH, NH₂, an amino acid fragment comprising 1-10 amino acids, a nucleotide fragment comprising 1-10 nucleotides;
Lk is L₁-L₂-L₃;
L₁, L₂, L₃, t, Y and W are as defined in formula (V), respectively.

In a preferable embodiment, in formula (V-1-1), x is selected from OH, NH₂ and Gly.

In a particular embodiment, in formula (V-1-1), both Y and W are absent, Lk is L₁-L₂-L₃, L₁ is —NH—, L₃ is —(CO)—, L₂ is —(C₂H₄—O)ᵢ—C₂H₄—, i=4, and the structure of the compound of formula (V-1-1) is as shown in the following formula (V-1-1-1):

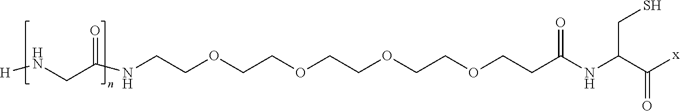

In a particular embodiment, in formula (V-1-1), W is absent, Y is L, L is leucine (Leu), Lk is L₁-L₂-L₃, L₁ is —NH—, L₃ is —(CO)—, L₂ is —(C₂H₄—O)ᵢ—C₂H₄—, i=4, and the structure of the linking unit is as follows (V-1-1-2):

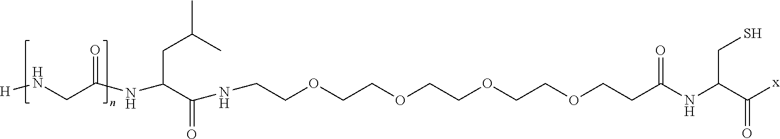

In yet a particular embodiment, in formula (V-1-1), W is absent, Y is Q, Q is glutamine (Gln), Lk is L₁-L₂-L₃, L₁ is —NH—, L₃ is —(CO)—, L₂ is —(C₂H₄—O)ᵢ—C₂H₄—, i=4, and the structure of the linking unit is as follows (V-1-1-3):

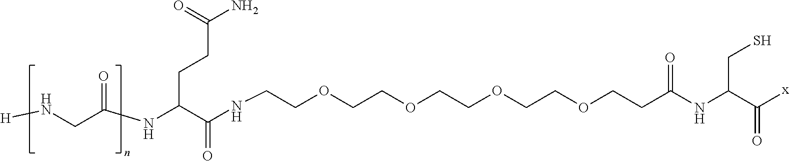

In a particular embodiment, in formula (V-1-1), both Y and W are absent, Lk is L₁-L₂-L₃, L₁ is —NH—, L₃ is —(CO)—, L₂ is —C₅H₁₀—, and the structure of the linking unit is as follows (V-1-1-4):

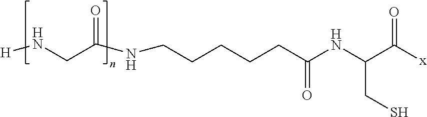

In yet a particular embodiment, in formula (V-1-1), both Y and W are absent, Lk is L₁-L₂-L₃, L₁ is —NH—, L₃ is —(CO)—, L₂ is —C₅H₁₀-group substituted with one —NR₁R₂ group, R₁ is hydrogen, R₂ is —(CO)CH₃, and the structure of the linking unit is as follows (V-1-1-5):

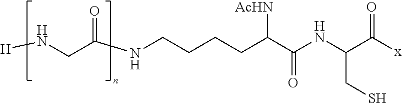

In some embodiments of the linking unit of formula (V-2), when t2 is 1, D2 is LPXTG and A1 is

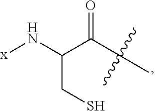

the structure of the compound of formula (V-2) is as shown in the following formula (V-2-1):

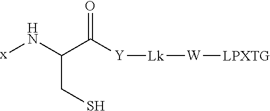

wherein x is selected from hydrogen, an amino acid fragment comprising 1-10 amino acids, a nucleotide fragment comprising 1-10 nucleotides;
Lk is L₁-L₂-L₃;
L₁, L₂, L₃, Y and W are as defined in formula (V), respectively.

In an embodiment, x is hydrogen.

In an embodiment, A1 and A2 are each independently a maleimide functional group. The maleimide functional group is introduced into the molecule of formula (V) by a suitable bifunctional cross-linking agent.

In a preferable embodiment, the bifunctional cross-linking agent for introducing maleimide functional group include, but are not limited to, N-succinimidyl 4-(N-maleimidomethyl)cyclohexane-1-carboxylate (SMCC), the “long chain” SMCC analogue N-[alpha-maleimidoacetoxy] succinimide ester (AMAS), N-gamma-Maleimidobutyryl-oxysuccinimideester (GMBS), 3-MaleiMidobenzoic acid N-hydroxysucciniMide ester (MBS), 6-maleimidohexanoic acid N-hydroxysuccinimide ester (EMCS), N-succinimidyl 4-(4-maleimidophenyl) butyrate (SMPB), Succinimidyl 6-[(beta-maleimidopropionamido) hexanoate (SMPH), Succinimidyl 4-(N-maleimidomethyl) cyclohexane-1-carboxy-(6-amidocaproate) (LC-SMCC), N-succinimidyl 11-(maleimido) undecanoate (KMUS), and a bifunctional crosslinking agent comprising N-hydroxy succinimide-(polyethyleneglycol alcohol)ₙ (SM (PEG)ₙ), wherein n indicated there are 2, 4, 6, 8, 12 or 24 polyethylene glycol (PEG) units. The exemplary maleimide functional groups introduced to A1 or A2 immediately after reacting with bifunctional cross-linking agent may be those listed in the following table:

| Bifunctional | Exemplary maleimide |
|---|---|
| cross-linking | functional group introduced to |
| agent | A1 or A2 |
| SMCC | 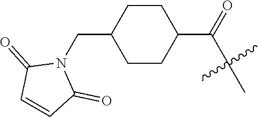 [SMILES: *C(=O)C1CCC(CN2C(=O)C=CC2=O)CC1] |
| | mcc |
| GMBS | 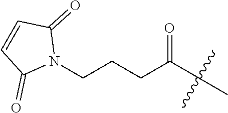 [SMILES: *C(=O)CCCN1C(=O)C=CC1=O] |
| EMCS | 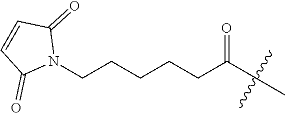 [SMILES: *C(=O)CCCCCN1C(=O)C=CC1=O] |
| | mc |
| SMPH | 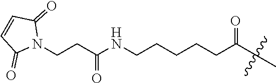 [SMILES: *C(=O)CCCCCNC(=O)CCN1C(=O)C=CC1=O] |
| KMUS | 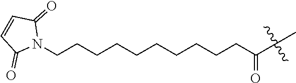 [SMILES: *C(=O)CCCCCCCCCCN1C(=O)C=CC1=O] |
| AMAS | 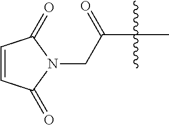 [SMILES: *C(=O)CN1C(=O)C=CC1=O] |
| MBS | 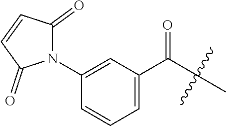 [SMILES: *C(=O)c1cccc(N2C(=O)C=CC2=O)c1] |
| SMPB | 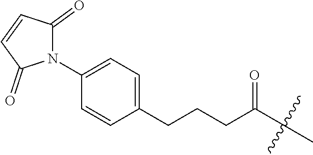 [SMILES: *C(=O)CCCc1ccc(N2C(=O)C=CC2=O)cc1] |
| LC -SMCC | 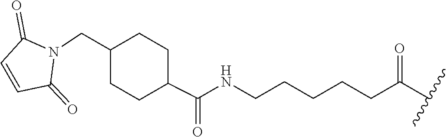 [SMILES: *C(=O)CCCCCNC(=O)C1CCC(CN2C(=O)C=CC2=O)CC1] |
| a | a maleimide functional group |
| bifunctional | comprising |
| crosslinking agent comprising SM (PEG)ₙ | 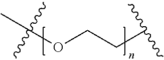 (PEG)ₙ |

In an embodiment, A1 and A2 are each independently selected from me and mcc.

In some embodiments of the linker of formula (V-1), wherein t2 is 1, D1 is Gₙ, G is glycine, A2 is mcc, W is absent, Lk is L₄, L₄ is an optionally derivatized lysine, and the structure of the compound of formula (V-1) is as shown in the following formula (V-1-2):

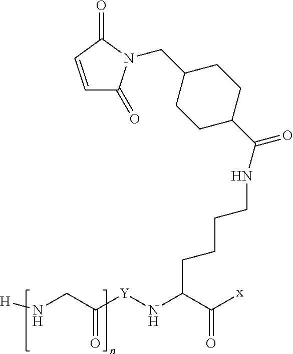

wherein n is an integer of 3 to 10;
x is selected from the group consisting of hydrogen, OH, NH₂, an amino acid fragment comprising 1-10 amino acids, a nucleotide fragment comprising 1-10 nucleotides;
Y is as defined in formula (V).

In an embodiment, in formula (V-1-2), Y is absent, and the structure of the linker is as follows (linker 2):

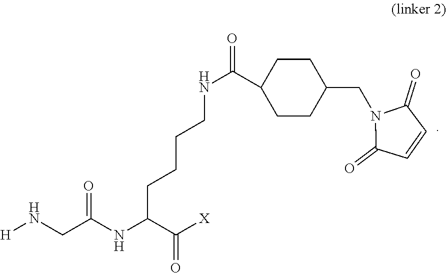

In a particular embodiment, in formula (V-1-2), Y is absent, n=3, x is OH, and the structure of the linker is as follows (linker LU104):

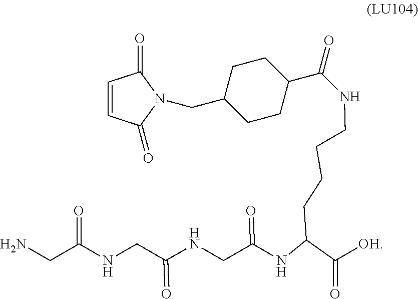 [SMILES: NCC(=O)NCC(=O)NCC(=O)NC(CCCCNC(=O)C1CCC(CN2C(=O)C=CC2=O)CC1)C(=O)O]

### Linker-Payload Compound of Endoglycosidase

Also provided is a linker-payload compound with formula (VI):

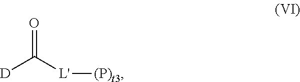

wherein
P′ is payload;
D-C(O)-L′- is linker;
D-C(O)- is a disaccharide structure

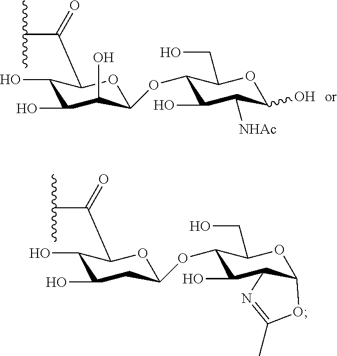

L′ is a connecting moiety. For example, it can be chemically (for example, by hydrolysis) or biologically (for example, by enzyme catalysis) disconnected from P′ to release P′. L′ is directly connected with carbonyl group in D-C(O)— via —NH— therein, wherein when L′ is a linear connecting moiety, it is connected with 1 P′, and t3 is 1, while when L′ is a branched connecting moiety, each branch can be connected with 1 P′, and t3 is an integer greater than 1 (for example, 2, 3, 4, 5, 6, 7, 8, 9 or 10).

### Payload

In the present disclosure, the payload may be selected from a small molecule compound (for example, a small molecule with various mechanisms of action, including various traditional small molecule drugs, a photoacoustic dynamic therapy drug, a photothermal therapy drug, etc., such as a chemotherapy drug, a small molecule targeted drug, an immune agonist, etc., for example, a traditional cytotoxic drug such as a cisplatin, paclitaxel, 5-fluorouracil, cyclophosphamide and bendamustine; a small molecule targeted drug, such as imatinib mesylate, gefitinib and anlotinib; an immune agonist, such as a STING agonist and a TLR agonist), a nucleic acid and a nucleic acid analogue, a tracer molecule (including a fluorescent molecule, a biotin, a fluorophore, a chromophore, a spin resonance probe and a radioactive label, etc.), a short peptide, a polypeptide, a peptidomimetic and a protein. In an embodiment, the payload is selected from the group consisting of small molecule compounds and nucleic acid molecules. In a preferable embodiment, the payload is selected from small molecular compounds. In a more preferable embodiment, the payload is selected from the group consisting of cytotoxins and fragments thereof.

In an embodiment, the payload is a cytotoxin or a fragment thereof, with optional derivatization to connect to the linker.

In an embodiment, the cytotoxin is selected from the cytotoxin is selected from the group consisting of taxanes, maytansinoids, auristatins, epothilones, combretastatin A-4 phosphate, combretastatin A-4 and a derivative thereof, a indole-sulfonamides, vinblastines such as vinblastine, vincristine, vindesine, vinorelbine, vinflunine, vinglycinate, anhydrovinblastine, dolastatin 10 and an analogue thereof, halichondrin B, eribulin, indole-3-oxoacetamide, podophyllotoxin, 7-diethylamino-3-(2′-benzoxazolyl)-coumarin (DBC), discodermolide, laulimalide, camptothecins and a derivative thereof, mitoxantrone, mitoguanidine hydrazone, nitrogen mustard, nitrosoureasm, aziridines, benzodopa, carboquone, meturedepa, uredepa, alacinomycin, actinomycin, antramycin, bleomycins, actinomycin C, carabicin, carminomycin, sarkomycin, actinomycin D, daunorubicin, detorubicin, adriamycin, epirubicin, esorbicin, idarubicin, macellomycin, mitomycins, nogalamycin, olivomycin, peplomycin, porfiromycin, puromycin, ferric adriamycin, rodorubicin, rufocromomycin, streptozocin, zinostatins, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, thiamiprine, thioguanine, ancitabine, gemcitabine, enocitabine, azacitidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and proteasome inhibitors. In an embodiment, the cytotoxin is selected from vinblastines, colchicines, taxanes, auristatins, maytansinoids, calicheamicin, doxorubicin, duocarmycin, SN-38, cryptophycin analogue, deruxtecan, duocarmazine, centanamycin, dolastansine, pyrrolobenzodiazepine and exatecan and a derivative thereof. In an embodiment, the cytotoxin is selected from auristatins, especially MMAE, MMAF or MMAD. In another embodiment, the cytotoxin is selected from exatecan and a derivative thereof, for example DX8951f. In an embodiment, the cytotoxin is selected from DXd-(1) and DXd-(2); preferably DXd-(1).

In an embodiment, the cytotoxin is maytansinoids, such as DM1, etc. It should be noted that when a cytotoxin containing a sulfhydryl moiety is used, the sulfhydryl moiety can react with a maleimide moiety to form a thiosuccinimide, such as maytansinoids, such as DM1, and the cytotoxin can be directly linked through the thiosuccinimide. In this case, it can be understood that in some embodiments, the payload and the sulfhydryl moiety together constitute a cytotoxin, so in this case, the payload represents the rest of the cytotoxin molecule except the sulfhydryl moiety.

In an embodiment, the cytotoxin is a compound of the following formula (i)

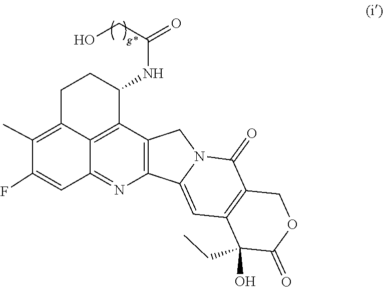

wherein, g is an integer of 1-6.

In an embodiment, g is an integer of 1-3, preferably 1.

In an embodiment, the cytotoxin is selected from the following compounds 1-15; wherein the wavy bond represents a linking site to the linker.

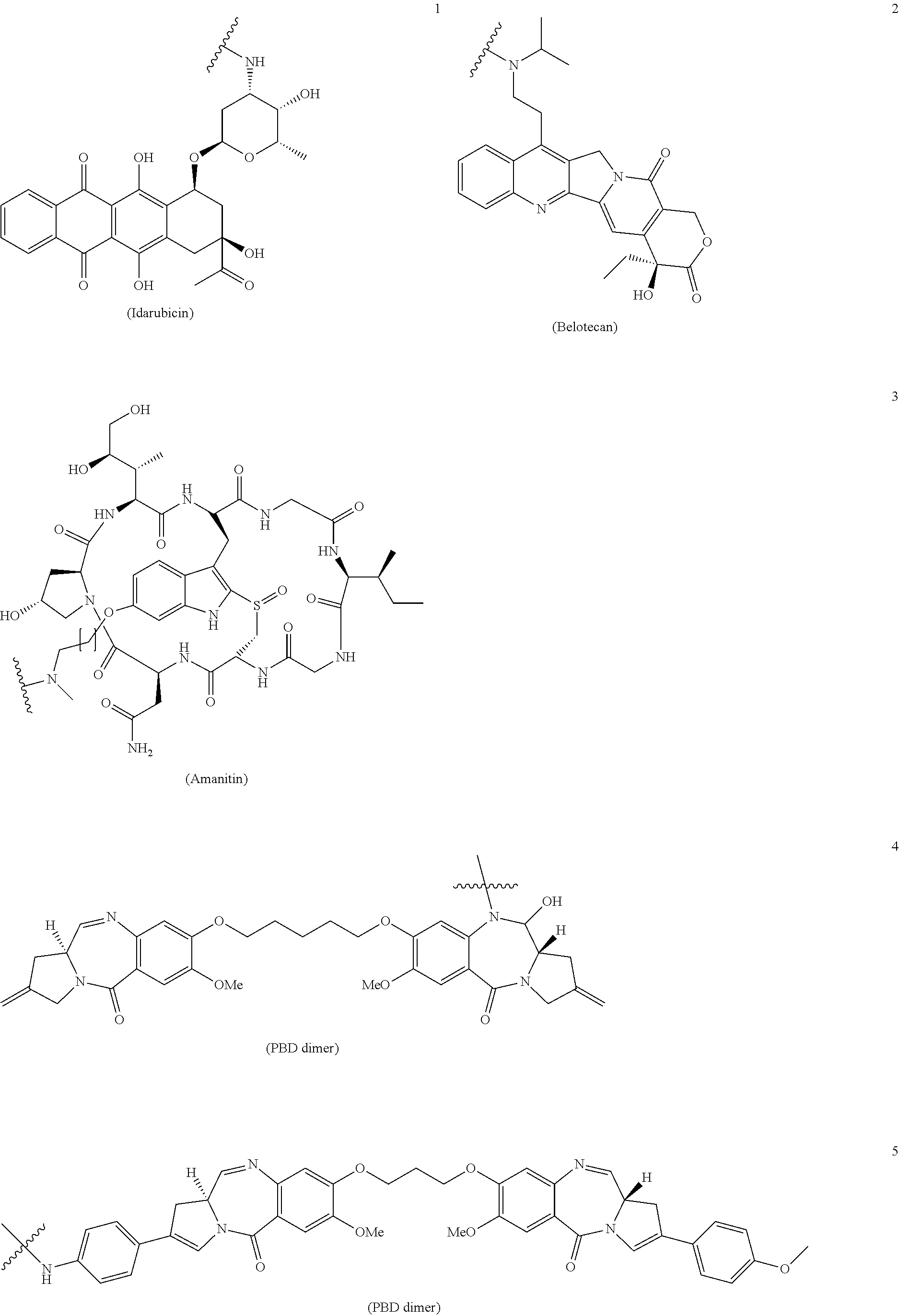

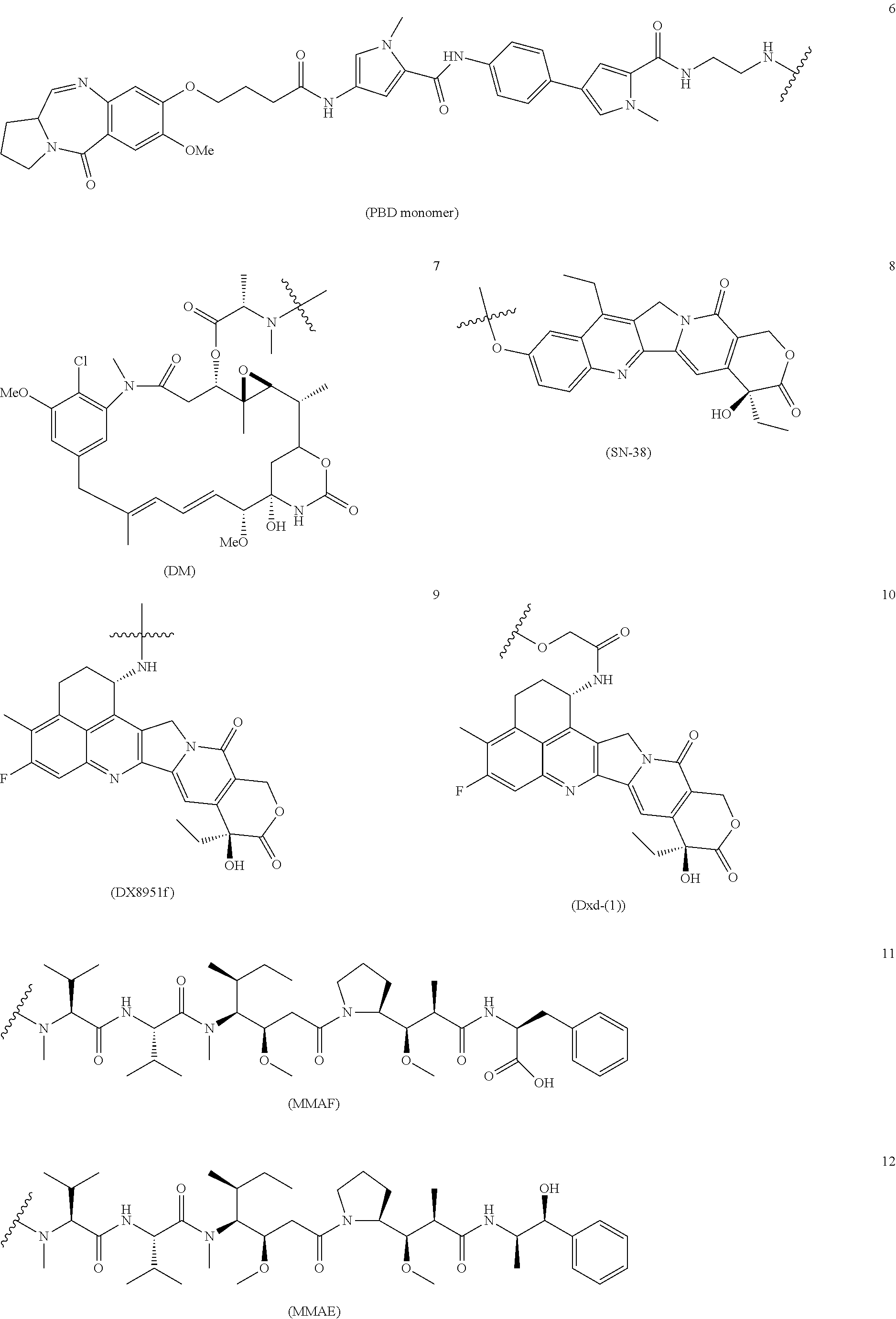

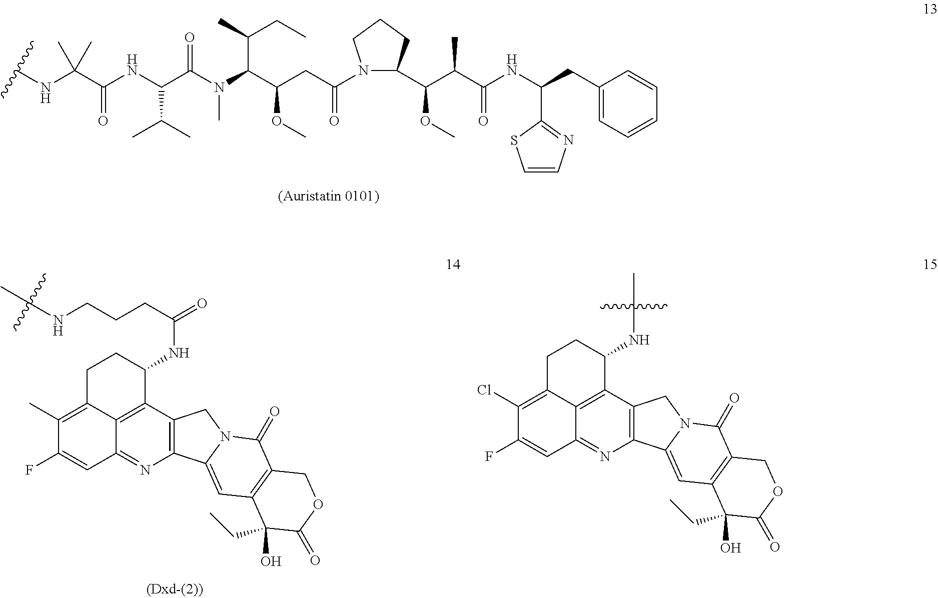

In some embodiments, the paylaod is selected from DX8951f, DXd-(1) and DXd-(2), and compound 15, preferably DX8951f or DXd-(1), more preferably DXd-(1).

In some embodiments, cytotoxin also includes those inhibiting cell proliferation. In some embodiment, cytotoxin is tyrosine kinase inhibitor (TKI). In some embodiments, cytotxin is selected from the group consisting of Lapatinib, Neratinib, Pyrroltinib, Afatinib, Gefitinib, Erlotinib and Osimertinib, or the derivatives thereof. In an embodiment, cytotoxin is Osimertinib. In a preferable embodiment, cycotoxin is a derivative of Osimertinib. In a preferable embodiment, cycotoxin has the structure of formula (A):

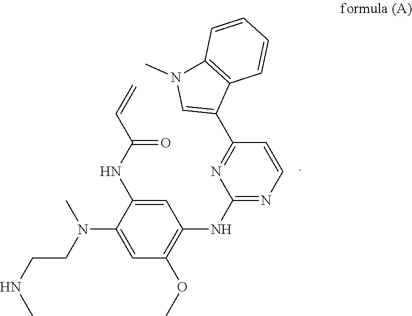 [SMILES: C=CC(=O)Nc1cc(Nc2nccc(-c3cn(C)c4ccccc34)n2)c(OC)cc1N(C)CCNC]

### Immunoligand

The immunoligand is selected from an antibody, a modified antibody format, an antibody derivative or fragment and/or a mimetic antibody. In some embodiments, the immunoligand contains an Fc terminus. In some embodiments, the immunoligand contains GlcNAc motif. In an embodiment, the immunoligand is an antibody selected from anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody and anti-CD123 antibody, Anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-GPNMB antibody, anti-ENPP3 antibody, anti-Nectin-4 antibody, anti-EGFRvIII antibody, anti-SLC44A4/AGS-5 antibody, Anti-CEACAM5 antibody, anti-PSMA antibody, anti-TIM1 antibody, anti-LY6E antibody, anti-LIV1 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, Anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin αV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H3/4 antibody, anti-TF (Tissue Factor) antibody, anti-ROR1/2 antibody; preferably anti-CD19 antibody, anti-ErbB2/HER2 antibody, anti-ErbB3/HER3 antibody, anti-CLDN18.2 antibody, anti-Nectin-4 antibody, anti-FGFR3 antibody and anti-Trop2 antibody; more preferably, anti-ErbB2/HER2 antibody and anti-ErbB3/HER3 antibody.

In an embodiment, anti-ErbB2/HER2 antibody comprises a light chain having the an amino acid sequence as shown in SEQ ID NO: 31 and/or a heavy chain having the the an amino acid sequence as shown in SEQ ID NO: 32.

In an embodiment, anti-ErbB3/HER3 antibody comprises a light chain having the an amino acid sequence as shown in SEQ ID NO: 33 and/or a heavy chain having the the an amino acid sequence as shown in SEQ ID NO: 34.

### Immunoligand/Payload Conjugate

In a further aspect, provided is the immunoligand/payload conjugate prepared by the process of the first aspect. In an embodiment, the immunoligand is antibody. In an embodiment, the payload is a drug. In an embodiment, the immunoligand/payload conjugate is an antibody-drug conjugate. In an embodiment, the immunoligand/payload conjugate is a dual-drug antibody-drug conjugate (dpADC). In an embodiment, the drug-antibody ratio (DAR) of the immunoligand/payload is 2-16, preferably 4-12, more preferably 4-8, and most preferably 6-8. In some embodiment, the first-drug-antibody ratio of the immuoligand/payload is 2-4. In some embodiment, the second-drug-antibody ratio of the immuoligand/payload is 2-4.

In some embodiments, the immunoligand/payload comprises an antibody having a first linker payload conjugated to the light chain C-terminal and a second linker-payload conjugated to the heavy chain Asn-297, wherein the payloads in the first linker payload and the second linker payload are different. In some embodiments, the immunoligand/payload comprises an antibody having a first linker payload conjugated to the heavy chain Asn-297 and a second linker-payload conjugated to the light chain C-terminal, wherein the payloads in the first linker payload and the second linker payload are different.

In some embodiments, the linker payload for forming the immunoligand/payload conjugate is conjugated to the antibody catalyzed by a transpeptidase or a variant thereof, such as sortase A, sortase B, sortase C, sortase D, sortase E or sortase F and a variant thereof, preferably sortase, more preferably sortase A and a variant thereof. In some embodiments, the linker payload for forming the immunoligand/payload conjugate is conjugated to the antibody catalyzed by the N-acetylglucosaminidase, for example, Endo H, Endo D, Endo F2, Endo F3, Endo M, Endo CC1, Endo CC2, Endo Om, Endo S and Endo S2, and a variant thereof.

In some embodiments, the antibody in the immunoligand/payload conjugate is an IgG; wherein the antibody binds ErbB2/Her2. In some embodiments, the antibody in the immunoligand/payload conjugate is modified with GALPETGG (SEQ ID NO: 60) to conjugate with the linker payload. In some embodiments, the antibody in the immunoligand/payload conjugate comprises an amino acid sequence of SEQ NO: 31.

In some embodiment, the antibody in the immunoligand/payload conjugate is an IgG; wherein the antibody binds ErbB3/Her3. In some embodiments, the antibody in the immunoligand/payload conjugate is modified with GALPETGG to conjugate with the linker payload. In some embodiments, the antibody in the immunoligand/payload conjugate comprises an amino acid sequence of SEQ NO: 33.

In some embodiments, the linker payload for forming the immunoligand/payload conjugate has the formula below:

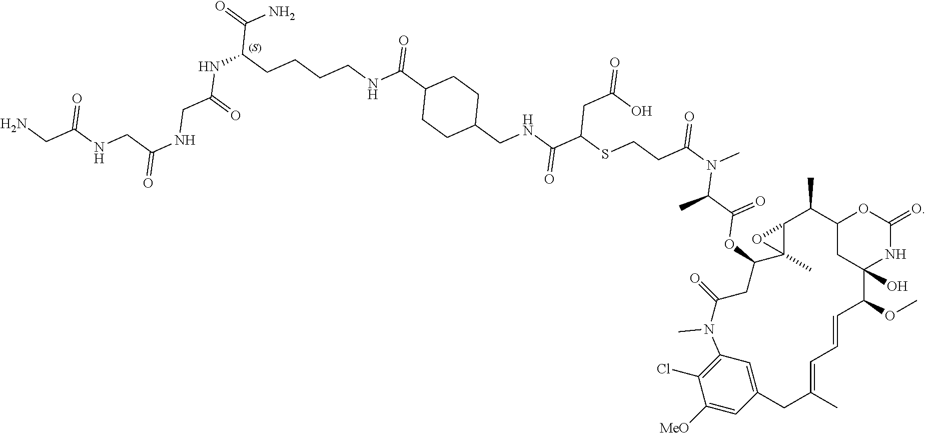

In some embodiments, the linker payload for forming the immunoligand/payload conjugate has the formula below:

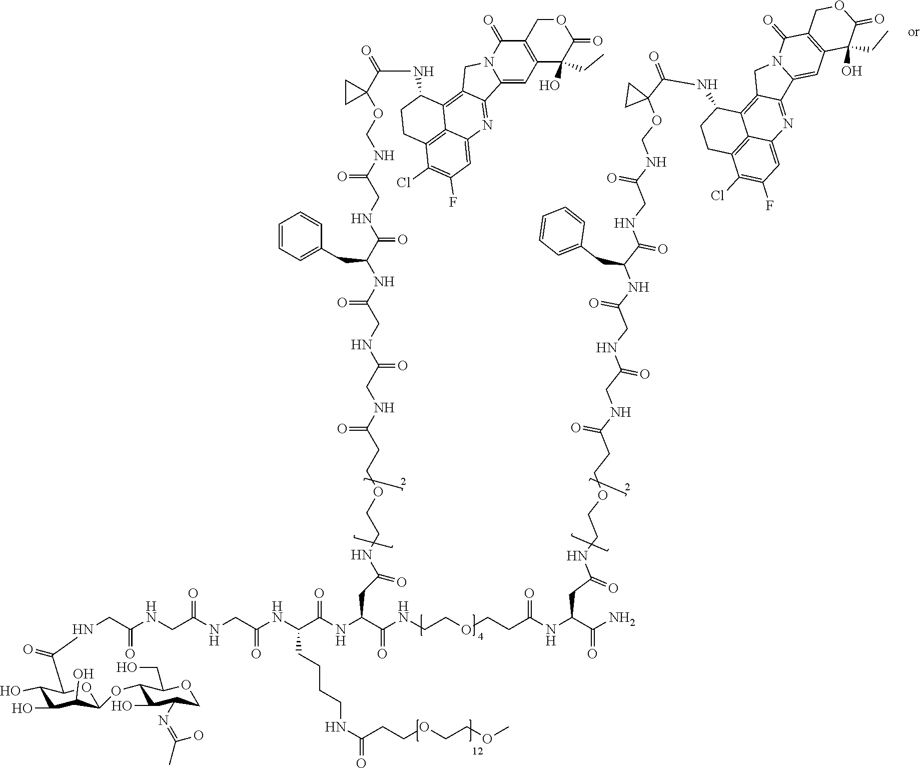

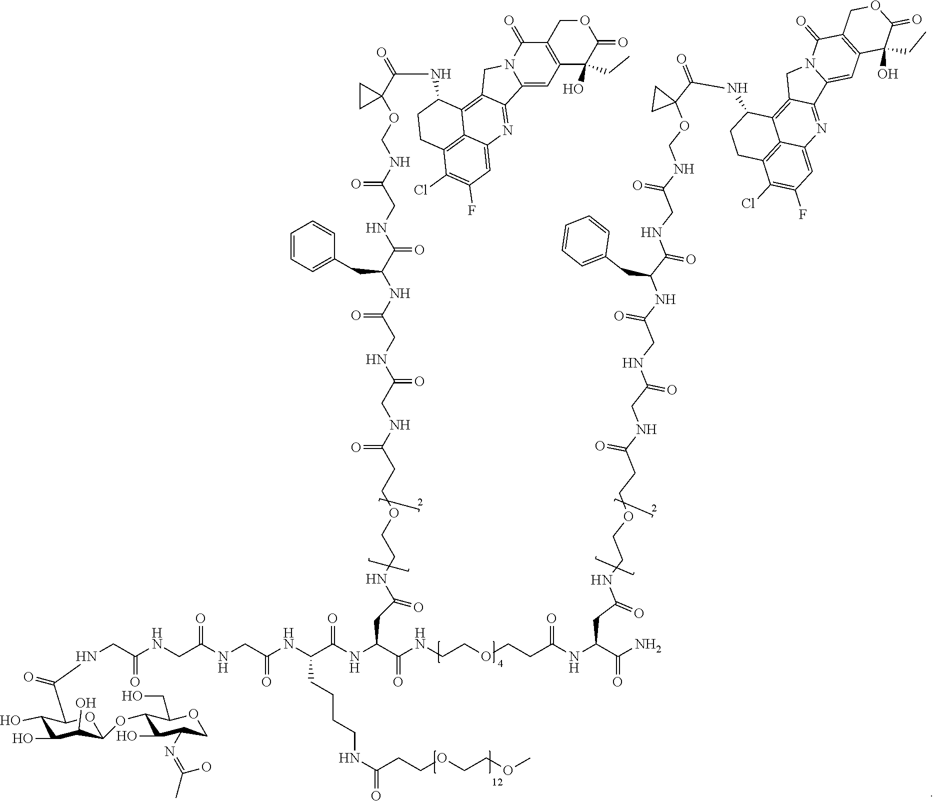

In some embodiments, the immunoligand/payload conjugate comprises an antibody having a linker payload conjugated to the light chain C-terminal, wherein the linker payload is conjugated to the antibody catalyzed by a transpeptidase or a variant thereof, such as sortase A, sortase B, sortase C, sortase D, sortase E or sortase F and a variant thereof, preferably sortase, more preferably sortase A and a variant thereof, and the linker payload has the formula below:

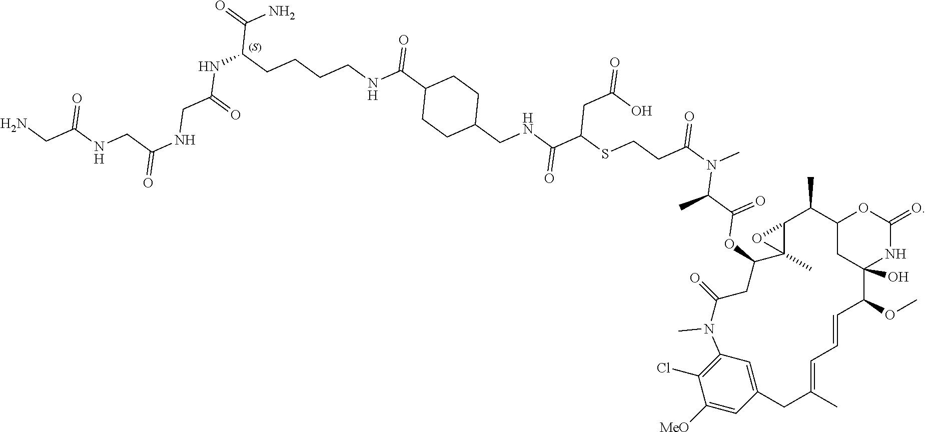

In some embodiments, the immunoligand/payload conjugate comprises an antibody having a linker payload conjugated to the heavy chain Asn-297, wherein the linker payload is conjugated to the antibody catalyzed by the N-acetylglucosaminidase, for example, Endo H, Endo D, Endo F2, Endo F3, Endo M, Endo CC1, Endo CC2, Endo Om, Endo S and Endo S2, and a variant thereof, and the linker payload has the formula below:

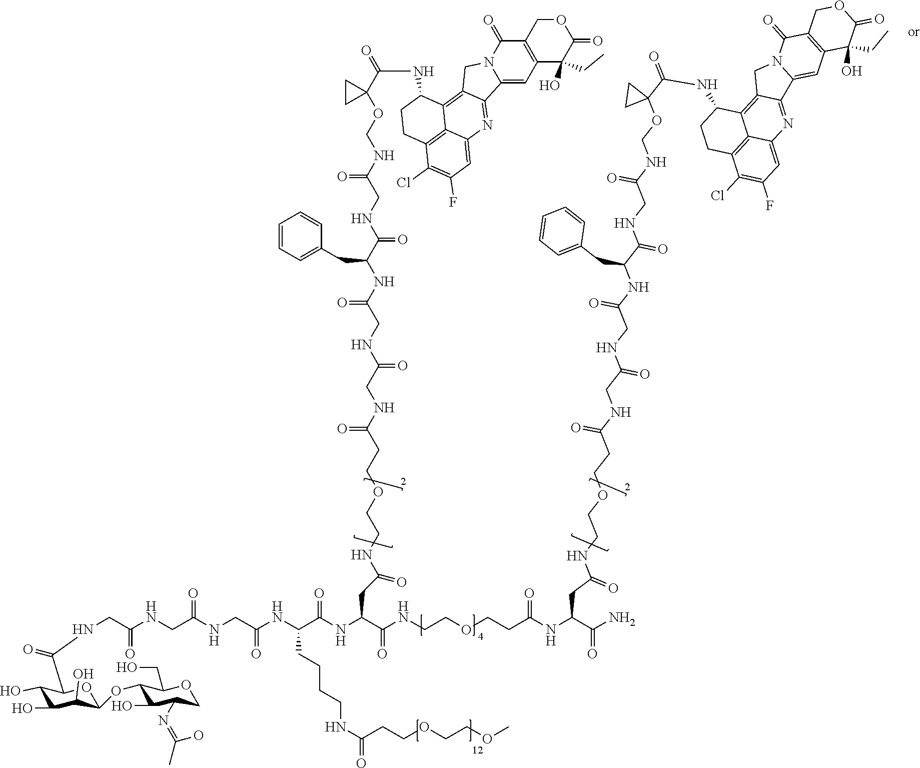

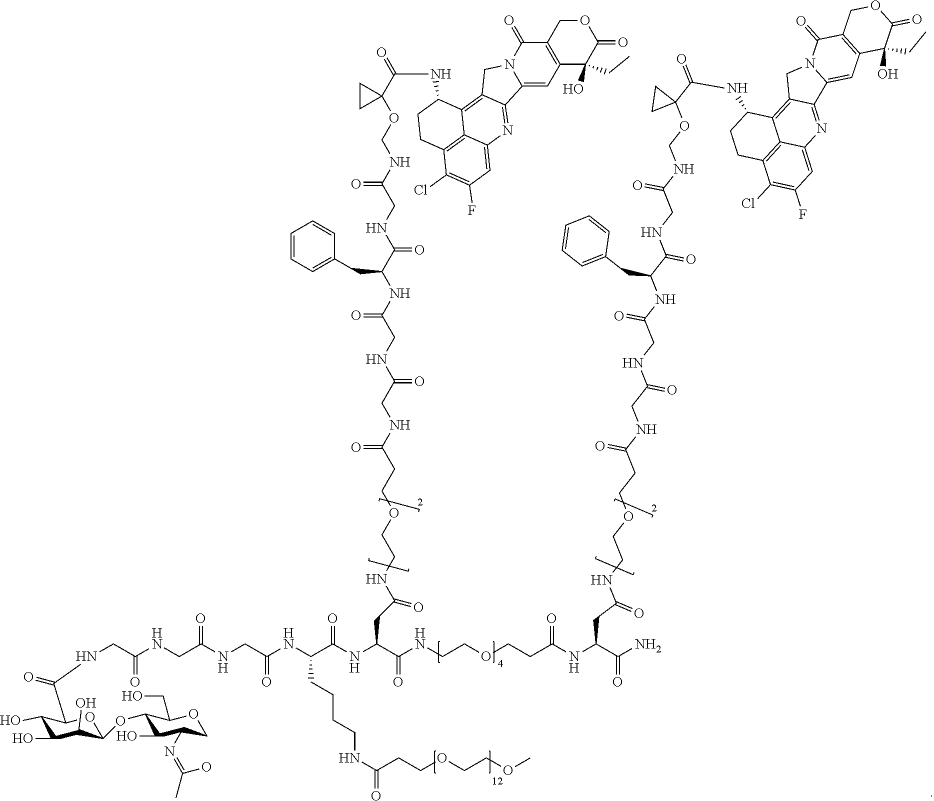

In some embodiments, the immunoligand/payload comprises an antibody having a first linker payload conjugated to the light chain C-terminal, wherein the first linker payload is conjugated to the antibody catalyzed by a transpeptidase or a variant thereof, such as sortase A, sortase B, sortase C, sortase D, sortase E or sortase F and a variant thereof, preferably sortase, more preferably sortase A and a variant thereof, and the first linker payload has the formula below:

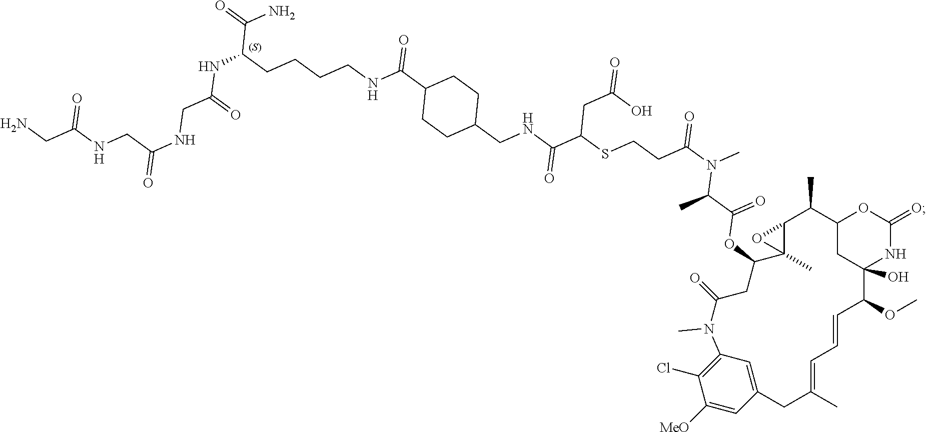

and a second linker-payload conjugated to the heavy chain Asn-297, wherein the second linker payload is conjugated to the antibody catalyzed by the N-acetylglucosaminidase, for example, Endo H, Endo D, Endo F2, Endo F3, Endo M, Endo CC1, Endo CC2, Endo Om, Endo S and Endo S2, and a variant thereof, and the second linker payload has the formula below:

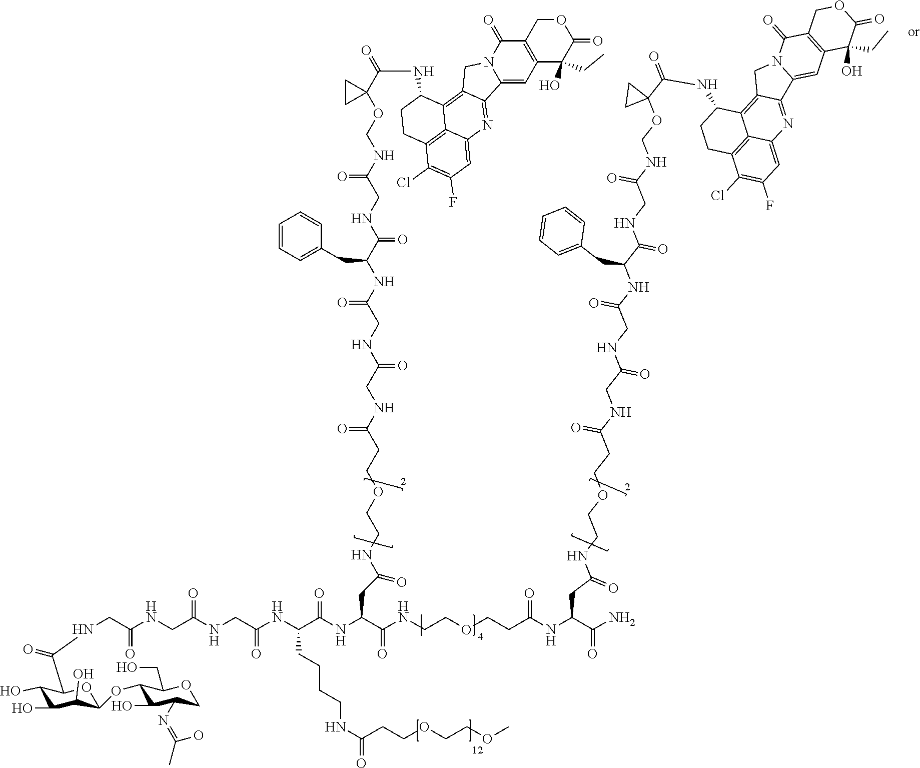

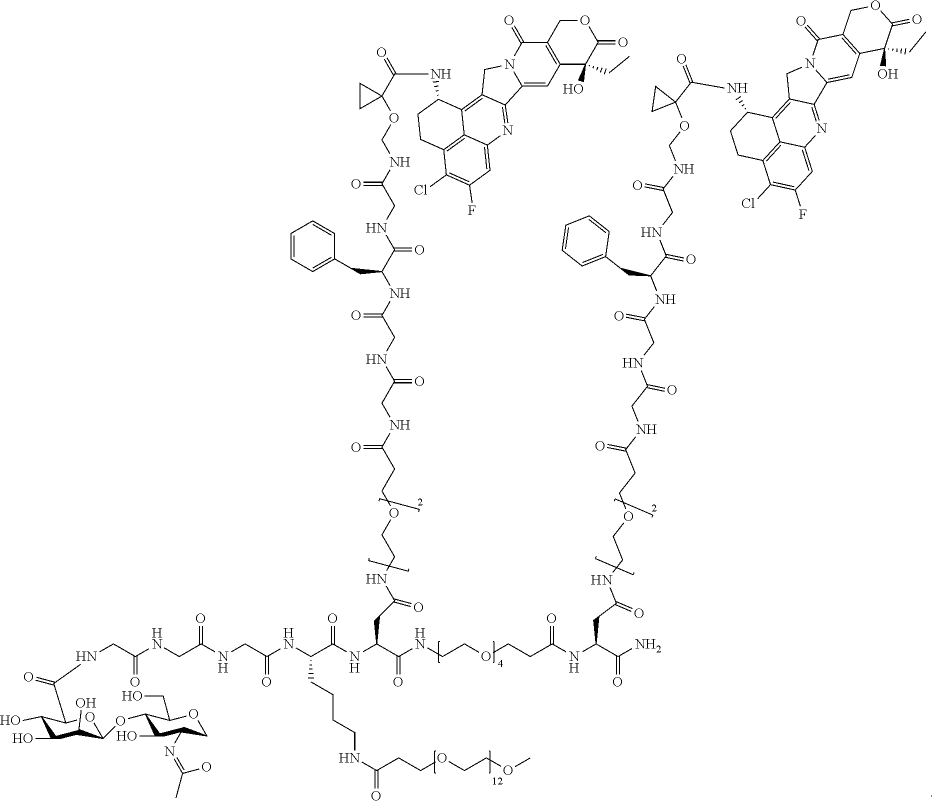

In some embodiments, the immunoligand/payload comprises an antibody having a first linker payload conjugated to the heavy chain Asn-297, wherein the first linker payload is conjugated to the antibody catalyzed by the N-acetylglucosaminidase, for example, Endo H, Endo D, Endo F2, Endo F3, Endo M, Endo CC1, Endo CC2, Endo Om, Endo S and Endo S2, and a variant thereof, and the first linker payload has the formula below:

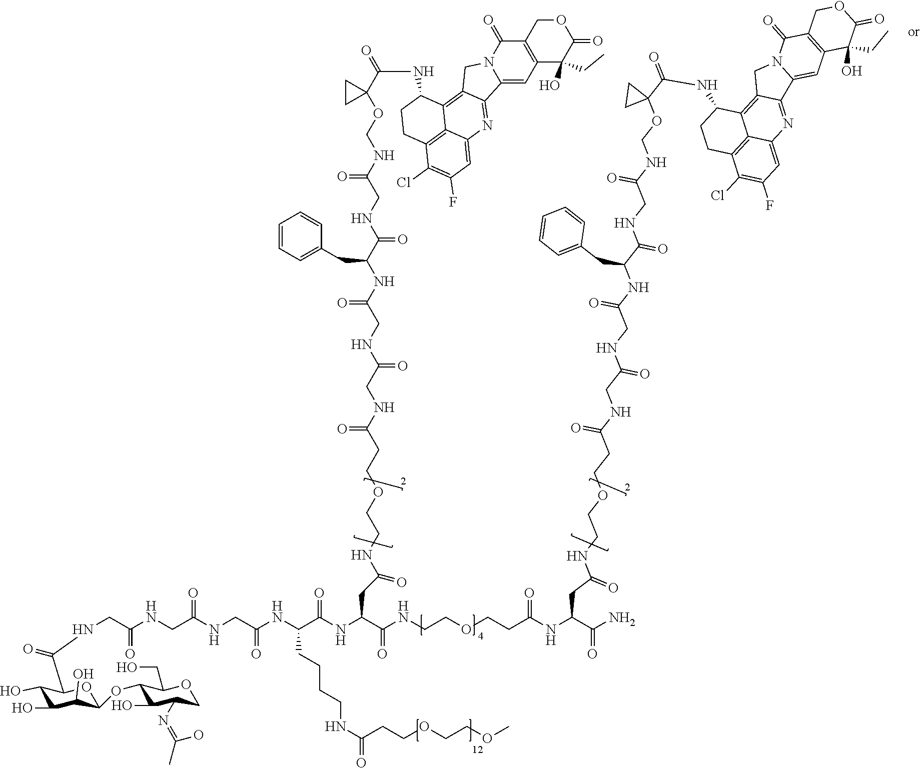

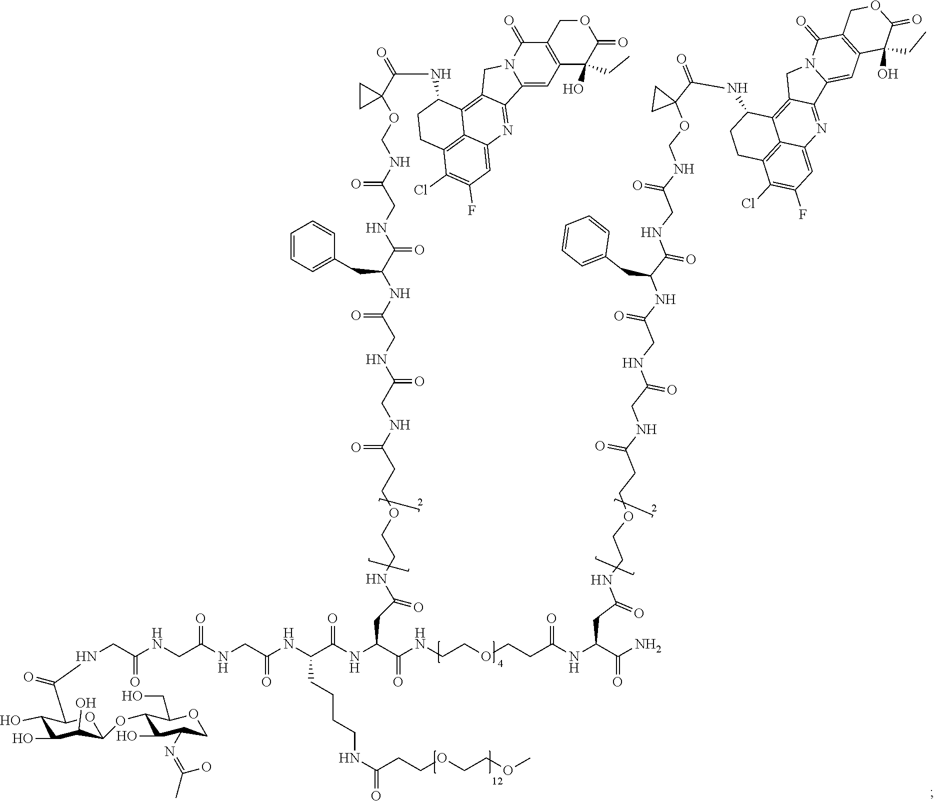

and a second linker-payload conjugated to the light chain C-terminal, wherein the second linker payload is conjugated to the antibody catalyzed by a transpeptidase or a variant thereof, such as sortase A, sortase B, sortase C, sortase D, sortase E or sortase F and a variant thereof, preferably sortase, more preferably sortase A and a variant thereof, and the second linker payload has the formula below:

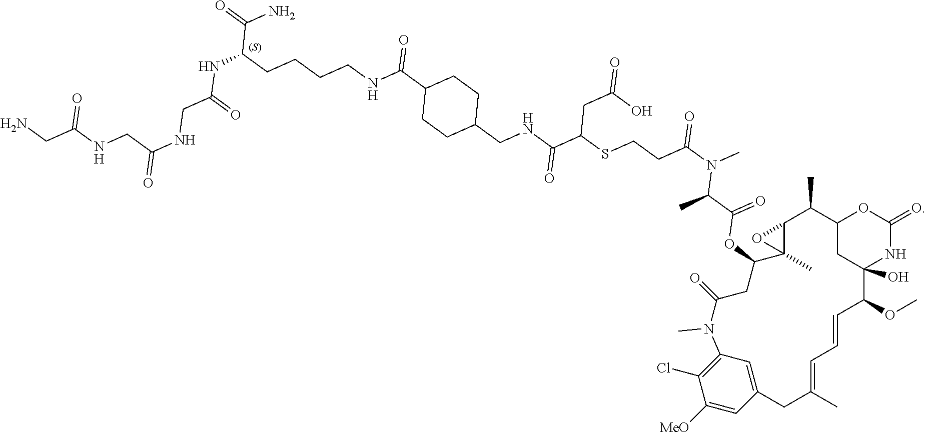

### Use of the Immunoligand/Payload Conjugate

In yet another aspect, provided is the use of immunoligand/payload conjugate by the process of the first aspect for the manufacture of a medicament for treating a proliferative disease. In an embodiment, the proliferative disease is selected from bladder cancer, breast cancer, cervical cancer, colorectal cancer, endometrial carcinoma, gastric cancer, head and neck neoplasm, renal cancer, liver cancer, lung cancer, ovarian cancer, prostate cancer, esophageal cancer, gallbladder cancer, pancreatic cancer, thyroid cancer, skin cancer, leukemia, multiple myeloma, chronic lymphocytic lymphoma, adult T-cell leukemia, B-cell lymphoma, acute myeloid leukemia, Hodgkin lymphoma or non-Hodgkin lymphoma, Waldenstrom macroglobulinemia, hairy cell lymphoma, cell lymphoma, Burkitt lymphoma, glioblastoma, melanoma or rhabdomyosarcoma.

### Beneficial Effects

The site-specific orthogonal bioconjugate modality resolved the technical problems listed below: the reported methods of constructing dpADC are mainly based on either dual chemical conjugation relay or enzymatic and chemical conjugation relay, both require a multi-step process, which might be CMC complicated and time consuming, and chemical conjugation often leads to uncertainty in conjugation site and a heterogenous DAR distribution, furthermore conjugates based on traditional cysteine-maleimide chemistry are also more liable to pre-mature release in systemic circulation due to the unstable linker design originated from the intact cysteine-maleimide ring structure. All these will cause problems in CMC manufacturing as well as safety concerns in clinical applications. To address this, we reported herein a highly site-specific, orthogonal enzymatic conjugation to provide homogenous dual payload antibody conjugates in a facile, efficient, single-step way.

Compared to the combination of two kinds of ADC drugs, the dual payload ADC can obviously reduce the occupation and competition of antigenic epitopes, and thus increase the potential efficacy and maximum dosage. The dual payload ADC can also be conveniently adminitrated regardless of difference of endocytosis efficiency, dosage ratio and interval for the two drugs to reach the target. As a single antibody modality is required for the loading of multiple drugs, the cost in antibody dosage can be greatly reduced. As the result, this dpADC platform and design can significantly improve the efficiency of drug candidate through manipulation of drug synergies, make the dpADC development cost-effective, and more importantly increase the success rate of drug development.

The dual-enzyme orthogonal conjugation technology platform is suitable for rapidly, efficiently and high-throughput preparing of candidate ADC molecules that target different targets and coupling various toxins with different linkers, so as to quickly screen out ADC drugs with novel structure and Mechanism of Action (MOA) that exhibit synergistic effects between toxins.

### EXAMPLES

Embodiments of the present disclosure are described in detail below. The embodiments described below are illustrative and are only used to explain the disclosure and cannot be understood as a limitation. Unless otherwise indicated, the proportion, percentage, etc. referred to herein are calculated by weight.

### Example 1. Preparation of Fusion Protein Enzymes

### 1.1 Preparation of Endoglycosidase Fusion Protein

### 1.1.1 Amino Acid Sequence of Halo-Endo S2-his

(SEQ ID NO: 30)
MAEIGTGFPFDPHYVEVLGERMHYVDVGPRDGTPVLFLHGNPTSSYVWR
NIIPHVAPTHRCIAPDLIGMGKSDKPDLGYFFDDHVRFMDAFIEALGLE
EVVLVIHDWGSALGFHWAKRNPERVKGIAFMEFIRPIPTWDEWPEFARE
TFQAFRTTDVGRKLIIDQNVFIEGTLPMGVVRPLTEVEMDHYREPFLNP
VDREPLWRFPNELPIAGEPANIVALVEEYMDWLHQSPVPKLLFWGTPGV
LIPPAEAARLAKSLPNCKAVDIGPGLNLLQEDNPDLIGSEIARWLSTLE
ISGGGGGSGGGGSMDKHLLVKRTLGCVCAATLMGAALATHHDSLNTVKA
EEKTVQTGKTDQQVGAKLVQEIREGKRGPLYAGYFRTWHDRASTGIDGK
QQHPENTMAEVPKEVDILFVFHDHTASDSPFWSELKDSYVHKLHQQGTA
LVQTIGVNELNGRTGLSKDYPDTPEGNKALAAAIVKAFVTDRGVDGLDI
DIEHEFTNKRTPEEDARALNVFKEIAQLIGKNGSDKSKLLIMDTTLSVE
NNPIFKGIAEDLDYLLRQYYGSQGGEAEVDTINSDWNQYQNYIDASQFM
IGFSFFEESASKGNLWFDVNEYDPNNPEKGKDIEGTRAKKYAEWQPSTG
GLKAGIFSYAIDRDGVAHVPSTYKNRTSTNLQRHEVDNISHTDYTVSRK
LKTLMTEDKRYDVIDQKDIPDPALREQIIQQVGQYKGDLERYNKTLVLT
GDKIQNLKGLEKLSKLQKLELRQLSNVKEITPELLPESMKKDAELVMVG
MTGLEKLNLSGLNRQTLDGIDVNSITHLTSFDISHNSLDLSEKSEDRKL
LMTLMEQVSNHQKITVKNTAFENQKPKGYYPQTYDTKEGHYDVDNAEHD
ILTDFVFGTVTKRNTFIGDEEAFAIYKEGAVDGRQYVSKDYTYEAFRKD
YKGYKVHLTASNLGETVTSKVTATTDETYLVDVSDGEKVVHHMKLNIGS
GAIMMENLAKGAKVIGTSGDFEQAKKIFDGEKSDRFFTWGQTNWIAFDL
GEINLAKEWRLFNAETNTEIKTDSSLNVAKGRLQILKDTTIDLEKMDIK
NRKEYLSNDENWTDVAQMDDAKAIFNSKLSNVLSRYWRFCVDGGASSYY
PQYTELQILGQRLSNDVANTLKDHHHHHHHHHH

Wherein Halo Tag is indicated by a single underscore and His Tag is indicated in italics.

### 1.1.2 Cloning of Halo-Endo S2-His

The nucleic acid sequence encoding Halo-Endo S2-His was synthesized by gene synthesis standard method and inserted into pET expression vector. Halo-Endo S2-His expression plasmid was transformed into Escherichia coli BL21(DE3). The corresponding antibiotics were added to LB (Luria-Bertani) culture medium, and cultured at 37° C. until OD600=0.5-1.0, and the bacteria were preserved.

### 1.1.3 Purification of Halo-Endo S2-His Tag (Bioreactor)

Glycerol bacteria were recovery and inoculated into LB liquid medium containing corresponding antibiotics for culture at 100-300 rpm and 37° C. for 2-8 h. When OD600 reached 0.5-1.0, it was inoculated into 10L bioreactor and cultured for 5-10 h. The expression was induced overnight by adding IPTG with final concentration of 0.2 mM at 16° C. The bacterial precipitation was collected by centrifugation at 2-8° C. for 10-30 min at a rotating speed of 3000-5000 rpm, and the bacteria were resuspended with equilibrition buffer and crushed under high pressure, and the supernatants and precipitates were separated by centrifugation at 2-8° C. for 10-60 min at a rotating speed of 5000-10,000 rpm.

Halo-Endo S2-His protein was purified by Ni affinity chromatography, Ni affinity column was connected to the protein purification system, and the purity water and equilibration buffer were used for full flushing. The supernatant sample was loaded by low-flowing. Equilibration buffer solution and 80 mM imidazole buffer solution were used respectively for washing, and Halo-Endo S2-His protein was eluted with 500 mM imidazole buffer. SDS-PAGE was used for analysis.

As shown in FIG. 1, Halo-Endo S2-His bounds to Ni affinity column, and there is no target protein in the flow-through solution, and Halo-Endo S2-His was eluted under the condition of 500 mM imidazole. The results show that Halo-Endo S2-His can be expressed by E. coli, cultured in a Bioreactor and purified by a Ni affinity column. Both upstream and down stream are easy to scale up.

### 1.2 Preparation of the Ligase Fusion Protein

### 1.2.1 Cloning and Purification of SrtA

Nucleic acids encoding the SrtA having an amino acid sequence selected from SEQ ID NO: 1-26 and their variants ([Ser34][Asnl00][Alal05][Thr136], SNAT; [Tyr34][Asnl00][Alal05][Thr136], YNAT; [Trp34][Asnl00][Aspl05][Thr136], WNDT; [Val34][Asnl00][Asnl05][Ser136], VNNS) were synthesized by gene synthesis standard method and sub-cloned into the expression vector pET-21a(+) at Ndel and EcoRI by Gibson Assembly. And a HiS₆ tag was inserted at the N-terminal of the SrtA open reading frame.

SrtA expression plasmids were transformed into E. coli BL21(DE3). Until OD600=0.5-0.8 after culturing at 37° C. in LB with 50 μg/ml Ampicillin, IPTG was added to a final concentration of 0.2 mM and sortase expression was induced at 25° C. for 12 hours. The target cells were harvested by centrifugation and re-suspended in lysis buffer (50 mM Tris pH 8.0, 300 mM NaCl) and then lysed by sonication and the supernatant was purified on Ni-NTA agarose following the manufacturer's instructions. The purity as judged by SDS-PAGE of the sortases were >90%. SrtAs' concentrations were calculated from the measured A280 using the extinction coefficient method.

### 1.2.2 Evaluation of Sortase Activities

Recombinant SrtAs as prepared in Example 1.2.1 were subjected to a fluorometric assay to measure their sortase activities. Reactions (total volume 100 μL, 0.085 mM Abz-LPETGK-Dnp, 18 mM tri-glycine in buffer A (buffer A: 5 mM CaCl₂), 150 mM NaCl, 50 mM Tris-HCl, pH 7.5) in 96-well plate were initiated at 37° C. by adding 0.625 μM purified SrtA or variants. Abz-LPETGK-Dnp was an internally quenched peptide with 2-aminobenzimidazole (Abz) as the fluorophore and 2,4-dinitrophenyl (Dnp) as the quencher. Upon cleavage of the peptide LPETGK by sortase, Dnp and Abz were separated and the fluorescent signal from Abz could be detected to indicate the sortase activity. The increase in fluorescence signal was continuously collected after 1 hr (λ_{exc}/λₑₘ=320 nm/420 nm, Gain-85, Biotek Cytation3 plate reader).

### 1.2.3 Cloning of the Ligase Fusion Protein (Halo-Sortase)

Nucleic acids encoding the ligase fusion proteins according to the present disclosure, each comprises a SrtA having an amino acid sequence selected from SEQ ID NO: 1-26 and their variants ([Ser34][Asn100][Ala105][Thr136], SNAT; [Tyr34][Asnl00][Ala105][Thr136], YNAT; [Trp34][Asnl00][Aspl05][Thr136], WNDT; [Val34][Asnl00][Asn105][Serl36], VNNS) and a Halo tag having the amino acid sequence of SEQ ID NO: 28 (Halo-Sortase), were cloned into a bacterial expression vector pET21a or pET24d. A Halo-Sortase having the amino acid sequence of SEQ ID NO: 29, which comprises a SrtA variant derived from Staphylococcus aureus (SEQ ID NO: 27) and a Halo tag (SEQ ID NO: 28), was used in the following examples.

### 1.2.4 Purification of Halo-Sortase

Halo-Sortase was expressed in E. coli BL21(DE3), purified and stored in 5%-10% glycerol at −80° C. His-Sortase with a His₆ tag and GBl-Sortase with a GB1 tag were prepared in a similar manner for comparison purposes.

### 1.2.5 Activity of Halo-Sortase

### Procedures:

(1) The purified ErbB2/Her2 antibody T-LCCT_{L}-HC (ErbB2/Her2 SEQ ID NO: 31 and HER2 SEQ ID NO: 32) was mixed with the linker-payload intermediate having the structure of (A) or (B) shown as FIG. 2 at an optimal molar ratio (Ab:linker-payload intermediate=1:1-1:100) in the conjugation buffer.
(2) Halo-Sortase, His-Sortase or GB1-Sortase prepared as in Example 1.2.4 was incubated with the mixture from step (1) at 4-40° C. for 0.5-20 h, respectively.
(3) The product from step (2) was stored at 4° C. or −80° C.
(4) The product was subjectd to 12% SDS-PAGE electrophoresis to determine the conjugation efficiency.

### Example 2. Preparation of Immobilized Enzymes

### 2.1 Preparation of the Chloroalkyl-linker Modified Resin (Chloro Resin)

Methods of preparing Chloro Resin have been described in, for example, in U.S. Pat. Nos. 7,429,472, 7,888,086 and 8,202,700, which are incorporated by reference herein in their entirety. Resins used for Chloro Resin preparation are shown in Table 2.

TABLE 2

| | Resin | NO. |
|---|---|---|
| 1 | NHS-activated Bestaresin 4FF | HX17091 |
| | (Highly crosslinked agarose, Bestchrom) | |
| 2 | CNBr-activated Bestaresin | HX17092 |
| | (Highly crosslinked agarose, Bestchrom) | |
| 3 | Epoxy-activated resin | HX17093 |
| | (Polymethyl methacrylate, Nano-Micro) | |
| 4 | Epoxy-activated Bestaresin 4FF | HX17094 |
| | (Highly crosslinked agarose, Bestchrom) | |

### Procedures:

(1) Pre-treatment

The resin was filtered in isopropyl alcohol and the filter cake was washed once with DMF, and dried by suctioning. The filter cake was transferred to a flask using DMF and stirred. Subsequently, ethylenediamine was added to the mixture, and stirred for 10 to 15 h. The filter cake was filtered and washed with DMF and then the liquid was drained.

For epoxy-activated resins:

The resin was filtered in isopropyl alcohol and the filter cake was washed once with H₂O, and dried by suctioning. The filter cake was transferred to a flask and stir. Subsequently, 25% to 28% concentrated ammonia water was added to the mixture, the system was slowly heated to 40-50° C. and reacted at 40-50° C. with stirring. Lowered the temperature of the system to 20-30° C., and filtered the mixture. The filter cake was washed with H₂O until the filtrate pH reached about 7-8. Subsequently, the filter cake was washed with DMF and the liquid was drained.
(2) The filter cake was transferred from the step (1) to a flask and stirred. Sequentially DMF containing chloroalkyl substrate having the structure of formula (I-1-1) as described above and triethylamine was added to the system. Reacted with stirring. Subsequently, the system was filtered, and the filter cake was washed with DMF and finally the liquid was drained.

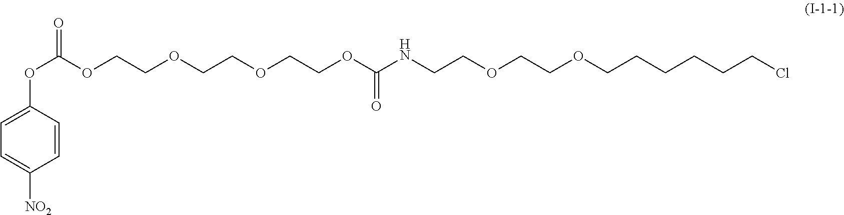 [SMILES: O=C(NCCOCCOCCCCCCCl)OCCOCCOCCOC(=O)Oc1ccc([N+](=O)[O-])cc1]

(3) The filter cake was transferred from step (2) to a flask. The stirring was turned on. Ac₂O and triethylamine were sequentially added to the mixture. Reacted with stirring. Then, the mixture was filtered and the filter cake was washed with DMF. Then the filter cake was washed with H₂O and the liquid was drained. Finally, the mixture was transferred to a container using 20% ethanol for storage.

Results: the Chloro Resin having the structure of formula (II-1) is obtained:

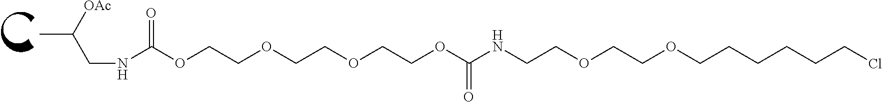 [SMILES: CC(=O)OC(C)CNC(=O)OCCOCCOCCOC(=O)NCCOCCOCCCCCCCl]

wherein the [symbol] is highly crosslinked agarose resin or polymethyl methacrylate resin.

### 2.2 Preparation Of Immobilized Halo-Endo S2-his

### 2.2.1 Immobilization of Halo-Endo S2-His on Chloro Resin

Halo-Endo S2-His was incubated with chloro resin at room temperature for 10 min-24 h, and the washing step was carried out with 20 mM Tris-HCl, 150 mM NaCl and pH 6.0-10.0 buffer, which was repeated for 3 times. The activity of immobilized endoglycosidase fusion protein resin (Halo-Endo S2-His covalently linked with chloro resin ligand) was tested. After passing the test, it was washed with 20 mM Tris-HCl, 150 mM NaCl, pH 6.0-10.0 buffer and finally stored at 2-8° C.

### 2.2.2 Immobilized Enzyme Load

1) 250 μl of chloro resins was taken to be tested, excessive glycosidase (Halo-Endo S2-His) was added, put on a rotary mixer, and immobilized for 2 h;
2) During the immobilization of Halo-Endo S2-His, the supernatant of the immobilized enzyme was taken at 15 min, 30 min, 1 h and 2 h. The resins solution was centrifuged at 3000 g for 3 min each time at room temperature, and the supernatant was aspirated and the concentration of the enzyme in the supernatant was determined using a Nanodrop UV spectrophotometer.
3) The endoglycosidase concentration at each time point was calculated, and the amount of immobilization at the timing moment of the enzyme was calculated by subtracting the concentration at each time point of the enzyme from the starting concentration, and the load change curve of the media immobilization Halo-Endo S2-His process was plotted.

SDS-PAGE detected the trend change of protein in the supernatant, as shown in FIG. 3. It is found that Halo-Endo S2-His in the supernatant becomes less and less over time, and is specifically immobilized on the chloro resin.

### 2.3 Preparation of Immobilized Halo-Sortase

### 2.3.1 Immobilization of Halo-Sortase to the Chloro Resin

### Procedures:

(1) The purified Halo-Sortase prepared as in Example 1.2.4 and the Chloro resin prepared as in Example 2.1 were incubated at room temperature for 10 min-24 h;
(2) The resin was washed with 20 mM Tris-HCl, 150 mM NaCl (pH 6.0-10.0) for three times;
(3) The enzymatic activity of the immobilized Halo-Sortase was determined;
(4) Optionally, the column was packed with the immobilized Halo-Sortase to obtain a Halo-Sortase column;
(5) The immobilized Halo-Sortase from step (3) or the Halo-Sortase column from step (4) was washed with 20 mMTns-HCl, 1-3 M NaCl (pH 6.0-10.0), 0.1-1.0 M NaOH and stored at 4° C.

### 2.3.2 Characterization of the Chloro Resin

### Procedures:

(1) 250 μl of each Chloro Resin was taken to be tested, in an excessive amount of Halo-Sortase was added, the tubes were placed on a rotor, and incubated for 2 h at room temperature;
(2) At different time points (15 min, 30 min, 1 h and 2 h, respectively) of the immobilization reaction, a drop of supernatant of each Chloro Resin was taken by centrifuging the tube at 3000 g for 3 min at room temperature, and the concentration of Halo-Sortase in the supernatant was determined using a Nanodrop spectrophotometer;
(3) The concentration of Halo-Sortase at each time point was calculated, which was then subtracted from the initial cencentration of Halo-Sortase to obtain the amount of immobilized Halo-Sortase at each time point, and a curve showing the amount of Halo-Sortase immoblized on the Chloro Resin as a function of the conjugation time was plotted.

### 2.3.3 Characterization of Immobilized Halo-Sortase

### Procedures:

(1) A fixed amount of Halo-sortase was immobilized onto the Chloro Resin as in Example 2.3.1.
(2) The immobilized enzyme was washed with 5-10 resin volumes of 1×Storage Buffer for three times, each time centrifuging at 3000 g for 3 min at room temperature to pellet the immobilized enzyme resin; the immobilized enzyme was resuspended in conjugation buffer.
(3) 25 μl of each immobilized enzyme resin was taken, 200 μl of GFP protein comprising a donor recognition motif LPETGG and small molecule reaction buffer containing the linker-payload intermediate were added, which comprised an acceptor recognition motif GGG conjugated to a small molecule compound, to start the conjugation reaction.
(4) At different time points (15 min, 30 min, 1 h and 2 h, respectively) of the conjugation reaction, a sample of the supernatant was taken for HIC-HPLC analysis.
(5) The conjugation efficiency was determined by HIC-HPLC.

### Example 3. Synthesis Of Linker-Payload

### 3.1 Synthesis of Disaccharide Compound 1

### 3.1.1 Synthesis of Compound 1c

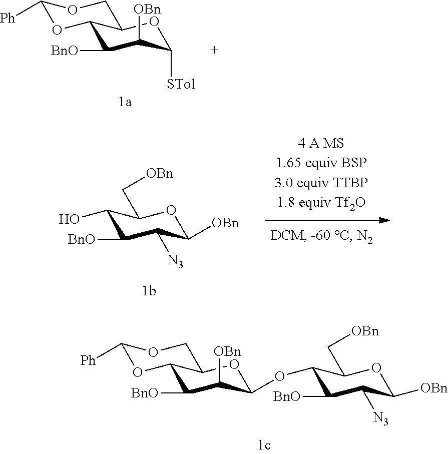

To an oven-dried 2-necked flask (100 mL) equipped with a Teflon-coated magnetic stir bar was added freshly activated 4 Å MS, capped with a sleeve stopper, the system was then baked with a heat gun under vacuum, cooled to ambient temperature, and then backfilled with nitrogen. Then Compound 1a (4.41 g, 7.96 mmol), anhydrous dichloromethane (30 mL) were added and stirred for 0.5 hour at room temperature (rt). Following the same protocol, Compound 1b (1.89 g, 3.98 mmol) was stirred and pre-dried in 20 mL anhydrous dichloromethane in the presence of activated molecular sieves. To the resulting reaction mixture of Compound 1a were added 1-(phenylthionyl) piperidine (BSP, 1.37 g, 6.56 mmol) and 2,4,6-tri-tert-butylpyrimidine (TTBP, 2.94 g, 11.94 mmol) at rt, and stirred for 20 minutes at the same temperature. Then the reaction flask was cooled to −65° C., followed by the dropwise addition of trifluoromethanesulfonic anhydride (1.2 mL, 7.16 mmol). After 2 minutes, the dichloromethane solution of the previously pre-dried Compound 1b was added to the system, and the resulting reaction mixture was stirred at −65° C. until the reaction was completed monitored by TLC (eluent: EtOAc/PE=1/8). Then the reaction mixture was quenched with saturated sodium bicarbonate solution (20 mL), extracted with dichloromethane (150 mL×3), the combined organic layer was washed with water and saturated salt water sequentially, dried over anhydrous sodium sulfate, filtered, concentrated under reduced pressure, and purified by flash column chromatography on silica gel (eluent: EtOAc/PE=1/12-1/10) to obtain Compound 1c as a colorless viscous oily liquid (2.72 g, 75.4% yield). ¹H NMR (400 MHz, chloroform-d) δ 7.55 (dd, J=7.6, 2.1 Hz, 2H), 7.51-7.28 (m, 28H), 5.60 (s, 1H), 5.14 (d, J=10.4 Hz, 1H), 5.00 (d, J=12.0 Hz, 1H), 4.94 (d, J=11.8 Hz, 1H), 4.90-4.80 (m, 2H), 4.76 (d, J=12.0 Hz, 1H), 4.74-4.63 (m, 3H), 4.57 (s, 1H), 4.48 (d, J=12.1 Hz, 1H), 4.37 (d, J=8.1 Hz, 1H), 4.22-4.10 (m, 2H), 4.05 (t, J=9.3 Hz, 1H), 3.80 (d, J=3.1 Hz, 1H), 3.72 (dd, J=11.2, 2.2 Hz, 1H), 3.67-3.52 (m, 3H), 3.49 (dd, J=9.8, 3.1 Hz, 1H), 3.41 (t, J=9.3 Hz, 1H), 3.35 (dt, J=9.8, 2.9 Hz, 1H), 3.16 (td, J=9.7, 4.8 Hz, 1H). MS (ESI) calcd for C₅₄H₅₆N₃O₁₀⁺ [M+H]⁺: 906.4. Found: 906.7.

### 3.1.2 Synthesis of Compound 1d

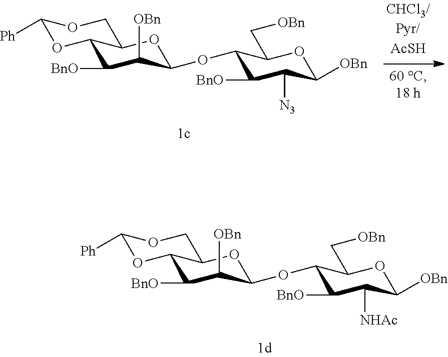

To a 25 mL Schlenk reaction flask were added Compound 1c (228 mg, 0.252 mmol), chloroform (1.2 mL), pyridine (1 mL), and AcSH (1.2 mL) successively. The tube was sealed and the system was stirred at 60° C. for 18 hours. Then the reaction system was cooled to rt, concentrated under reduced pressure to remove most of the solvents, and then water (30 mL) was added, the resulting mixture was quenched with ethyl acetate (20 mL×3), the combined organic layer was washed sequentially with saturated sodium bicarbonate solution (30 mL), 1 M hydrochloric acid (10 mL×4) and saturated sodium bicarbonate solution (30 mL). The resulting organic phase was dried over anhydrous sodium sulfate, concentrated and purified by flash column chromatography on silica gel (eluent: EtOAc/PE=1/10-1/1/) to offord Compound 1d as a white solid (200 mg, 86% yield). ¹H NMR (400 MHz, chloroform-d) δ 7.57-7.49 (m, 2H), 7.48-7.39 (m, 5H), 7.39-7.24 (m, 23H), 5.80 (d, J=8.1 Hz, 1H), 5.59 (s, 1H), 5.00 (d, J=6.7 Hz, 1H), 4.97-4.87 (m, 3H), 4.87-4.75 (m, 2H), 4.69-4.59 (m, 4H), 4.57 (s, 1H), 4.45 (d, J=12.0 Hz, 1H), 4.19-4.05 (m, 3H), 3.96 (t, J=7.3 Hz, 1H), 3.85-3.76 (m, 2H), 3.73-3.59 (m, 4H), 3.49 (dd, J=9.8, 3.1 Hz, 1H), 3.18 (td, J=9.7, 4.8 Hz, 1H), 1.77 (d, J=1.2 Hz, 3H). MS (ESI) calcd for C₅₆H₆₀NO₁₁⁺ [M+H]⁺: 922.4. Found: 922.4.

### 3.1.3 Synthesis of Compound 1e

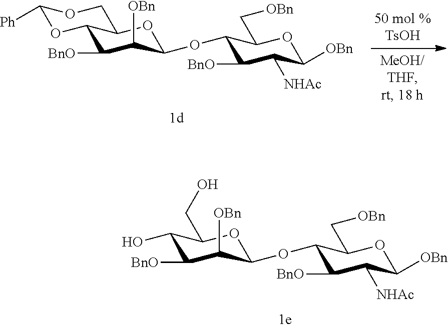

To a solution of Compound 1d (168 mg, 0.168 mmol) in methanol/tetrahydrofuran (1:1, 10 mL) was added p-toluenesulfonic acid monohydrate (35 mg, 0.168 mmol), the resulting mixture was stirred overnight at rt. After completion of the reaction, the mixture was quenched with saturated sodium bicarbonate solution (20 mL), extracted with dichloromethane (20 mL×3), the combined organic layer was dried over anhydrous sodium sulfate, filtered, concentrated under reduced pressure, and purified by flash column chromatography on silica gel (eluent: MeOH/DCM=1/20) to give Compound 1e as a white solid (136 mg, yield 89%). ¹H NMR (400 MHz, chloroform-d) δ 7.43-7.24 (m, 29H), 5.76 (d, J=7.9 Hz, 1H), 5.01 (d, J=6.9 Hz, 1H), 4.94 (dd, J=11.8, 2.3 Hz, 2H), 4.85 (d, J=11.7 Hz, 1H), 4.77 (d, J=11.8 Hz, 1H), 4.70-4.59 (m, 3H), 4.58-4.47 (m, 3H), 4.36 (d, J=11.7 Hz, 1H), 4.20 (t, J=7.9 Hz, 1H), 3.94 (t, J=7.5 Hz, 1H), 3.91-3.80 (m, 3H), 3.80-3.71 (m, 3H), 3.63-3.50 (m, 2H), 3.22-3.14 (m, 2H), 1.78 (s, 3H). MS (ESI) calcd for C₄₉H₅₆NO₁₁⁺ [M+H]⁺: 834.4. Found 834.5.

### 3.1.4 Synthesis of Compound 1f

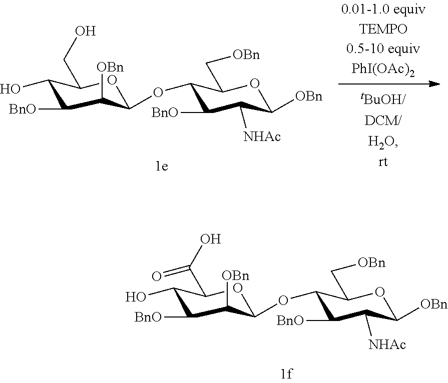

To a round falsk (100 mL) were added Compound 1e (130 mg, 0.156 mmol), dichloromethane, tert-butanol, water, iodobenzene diacetate (0.5-10 eq.) and 2,2,6,6-tetramethyl-1-piperidine oxide (0.01-1 eq.), the resulting reaction mixture was stirred overnight at rt. Upon completion, the reaction mixture was extracted with dichloromethane, and the combined organic phases were dried with anhydrous sodium sulfate, filtered, concentrated and purified by flash column chromatography on silical gel to give Compound 1f as a white solid (100 mg, 75.1% yield). ¹H NMR (400 MHz, chloroform-d) δ 7.42-7.31 (m, 18H), 7.30-7.27 (m, 2H), 7.25-7.20 (m, 3H), 7.15 (dd, J=6.7, 2.9 Hz, 2H), 6.06 (d, J=8.3 Hz, 1H), 4.96 (d, J=6.6 Hz, 1H), 4.93 (d, J=11.9 Hz, 1H), 4.86 (d, J=11.1 Hz, 1H), 4.81 (d, J=12.1 Hz, 1H), 4.73 (d, J=11.1 Hz, 1H), 4.71-4.65 (m, 3H), 4.65-4.57 (m, 3H), 4.49 (s, 1H), 4.46 (d, J=12.0 Hz, 1H), 4.23 (t, J=7.1 Hz, 1H), 4.15 (t, J=9.5 Hz, 1H), 3.89 (t, J=6.5 Hz, 1H), 3.86-3.65 (m, 6H), 3.54 (d, J=9.7 Hz, 1H), 3.30 (dd, J=9.3, 2.8 Hz, 1H), 1.65 (s, 3H). MS (ESI) calcd for C₄₉H₅₂NO₁₂⁻ [M−H]⁻: 846.3. Found: 846.3.

### 3.1.5 Synthesis of Compound 1

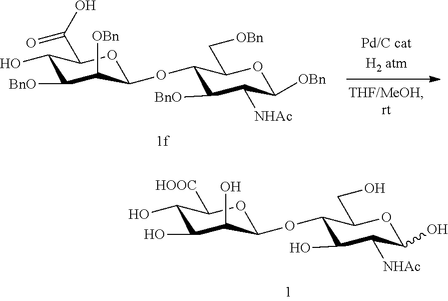

To a solution of Compound 1f (160 mg, 0.189 mmol) in tetrahydrofuran/methanol (1:1, 10 mL) was added palladium on carbon (10 wt. % loading), H₂ was bubbled through the solution for 5 mins, then the mixture was stirred under hydrogen atmosphere. Upon completion, the reaction mixture was filtered through Celite and washed with Methanol, the filtrate was then concentrated under reduced pressure, and dried in high vacuum to afford Compound 1 as a white solid (75 mg, 100% yield). MS (ESI) calcd for C₁₄H₂₂NO₁₂⁻ [M−H]⁻: 396.1. Found: 396.1.

### 3.2 Synthesis of Compound 2

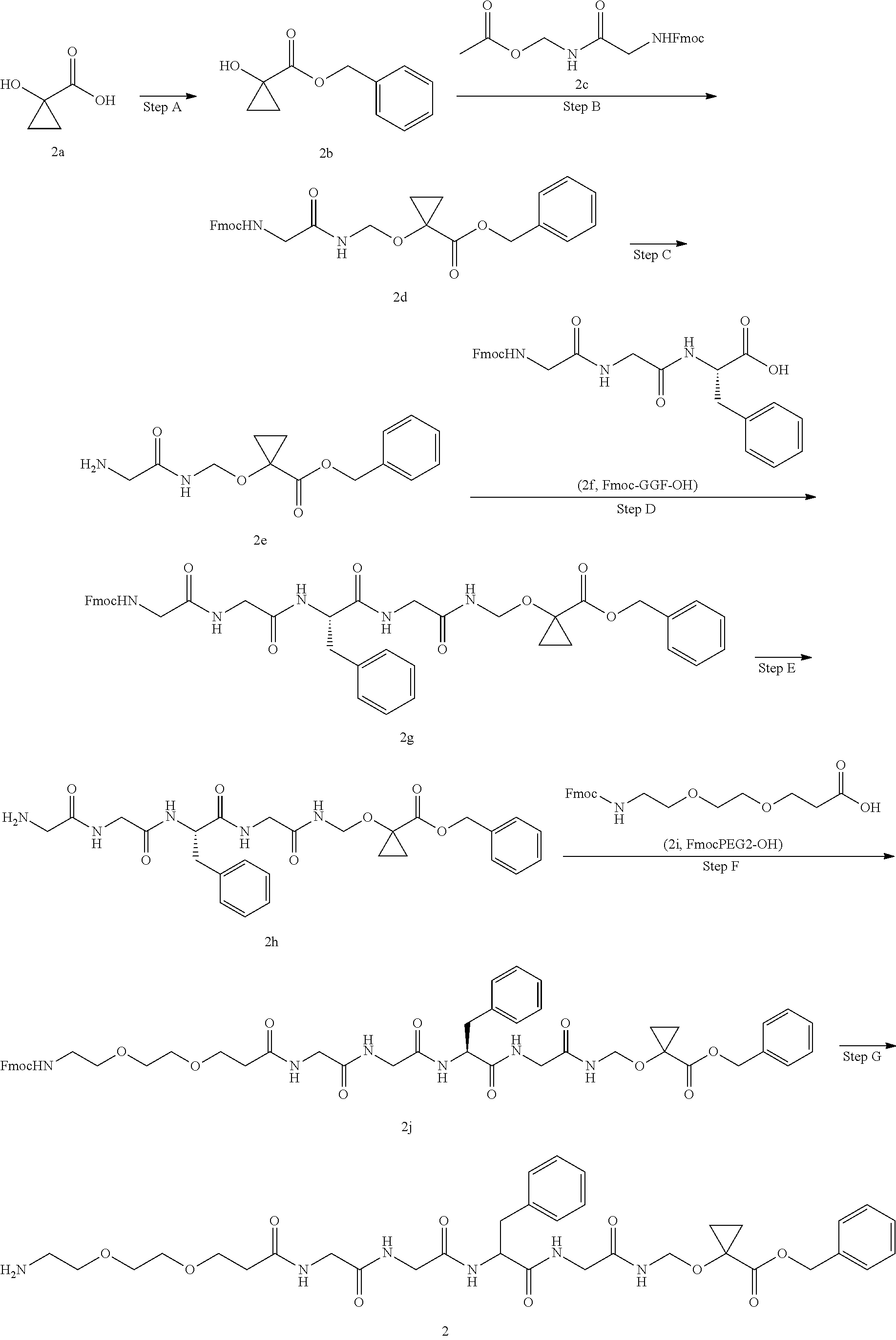

### Step A: Synthesis of Compound 2b

To a stirred solution of Compound 2a (1 equiv) in DMF (5 v/v) was added DIEA (3 equiv) dropwise under nitrogen atmosphere at 0° C., the resulting mixture was stirred at 0° C. for 10 min. Then benzyl bromide (1.3 equiv) was added dropwise, the reaction was then allowed to naturally warm to room temperature and stirred for another 16 hours. The reaction mixture was then slowly poured into ice water, extracted with MTBE (4 times), the combined organic layer was washed with saturated brine, dried over anhydrous sodium sulfate, filtered and concentrated under vacuum, purified by flash column chromatography on silical gel (elution:PE/EA=6:1) to yield Compound 2b as a light yellow oil (quantative yield).

### Step B: Synthesis of Compound 2d

To the solution of Compound 2b (2.0 equiv), Compound 2c (1 equiv) in THF (10 v/v) was added TsOH (0.1 equiv), the resulting mixture was stirred at rt for 4 h. Then the reaction solution was slowly poured into ice water, extracted 3 times with EA, the combined organic layer was washed with saturated aqueous sodium bicarbonate solution, water and saturated brine successively, the organic phase was dried over anhydrous sodium sulfate, filtered and concentrated and purified by flash column chromatography on silical gel (elution: PE/EA=1:1) to give Compound 2d as a white solid (40% yield).

### Step C: Synthesis of Compound 2e

To a stirred solution of Compound 2d in DMAc (10 v/v) was added DBU (0.5 equiv) dropwise at 15° C., the reaction mixture was stirred at the same temperature for 1.5 h. Upon completion, the reaction mixture was cooled down to 0° C., crude product Compound 2e was directly used in the next step without any purification.

### Step D: Synthesis of Compound 2g

To the reaction mixture in the last step was added PPTS (0.5 equiv), EDCI (1 equiv), HOBT (1 equiv) and Compound 2f (0.85 equiv) at 0° C., the resulting mixture was allowed to stir at 0° C. for 3-4 h. Upon completion, the reaction mixture was treated with ice water, extracted with 2-methyltetrahydrofuran (3 times). The combined organic layer was washed with 0.5 M hydrochloric acid, saturated aqueous solution of NaHCO₃, water and brine successively, dried over anhydrous sodium sulfate, filtered, concentrated and purified by flash column chromatography on silical gel (elution: DCM/MeOH) to yield Compound 2g as a white solid (78% yield).

### Step E: Synthesis of Compound 2h

To a stirred solution of Compound 2g in DMAc (10 v/v) was added DBU (0.5 equiv) dropwise at 15° C., the reaction mixture was stirred at the same temperature for 1.5 h. Upon completion, the reaction was cooled to 0° C., crude product Compound 2 h was directly used in the next step without any purification.

### Step F: Synthesis of Compound 2j

To the reaction mixture in the last step was added PPTS (0.5 equiv), EDCI (1 equiv), HOBT (1 equiv) and Compound 2i (0.85 equiv) at 0° C., the resulting mixture was allowed to stir at 0° C. for 3-4 h. Upon completion, the reaction mixture was treated with ice water, extracted with 2-methyltetrahydrofuran (3 times). The combined organic layer was washed with 0.5 M hydrochloric acid, saturated aqueous solution of NaHCO₃, water and brine successively, dried over anhydrous sodium sulfate, filtered, concentrated and purified by flash column chromatography on silical gel (elution: DCM/MeOH) to yield Compound 2j as a white solid (50% yield).

### Step G: Synthesis of Compound 2

To a stirred solution of Compound 2j in DCM (15 v/v) was added DBU (0.5 equiv) dropwise at 20° C. under nitrogen atmosphere, the reaction was stirred at the same temperature for 5 h. Upon completion, the reaction solution was diluted with DCM and sent to flash column chromatography on silical gel (eluent: DCM/MeOH) to afford Compound 2 as a white solid (82% yield).

### 3.3 Synthesis of Compound 3

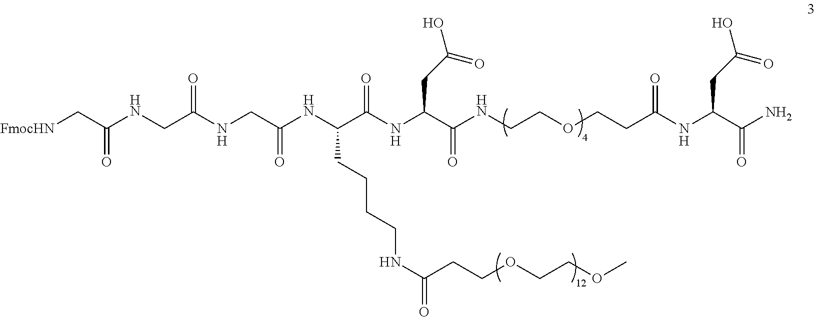

### Step 3.3.1 Synthesis of NH₂-Asp(OtBu)-Rink Amide Resin

400 g of Rink amide resin was weighed and fully swelled by 2400 mL of DCM. 2400 mL of deprotection reagent was added to remove Fmoc completely and then washed several times with DMF and DCM in room temperature. In the subsequent ninhydrin test, the resin was blue.

88.87 g Fmoc-Asp(OtBu)-OH and 29.19 g HOBT were weighed and dissolved in 2000 mL DMF and 80 mL DIC solution. After being placed in an ice bath at −10° C. for 0.5 h, it was slowly added into the reaction kettle with resin, and the reaction was stirred at room temperature for 2-5 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 3.3.2 Synthesis of NH₂-PEG4-Asp(OtBu)-Rink Amide Resin

131.64 g Fmoc-PEG4-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 3.3.3 Synthesis of NH₂-Asp(OtBu)-PEG4-Asp(OtBu)-Rink Amide Resin

222.18 g Fmoc-Asp(OtBu)-OH and 72.96 g HOBT were weighed and dissolved in 2000 mL DMF and 80 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 3.3.4 Synthesis of Dde-Lys (NH₂)-Asp(OtBu)-PEG4-Asp(OtBu)-Rink Amide Resin

191.75 g Dde-Lys(Fmoc)-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

2400 mL of deprotection reagent was added to remove Fmoc completely and then washed several times with DMF and DCM in room temperature. In the subsequent ninhydrin test, the resin showed blue color.

### Step 3.3.5 Synthesis of Dde-Lys (mPEG12)-Asp(OtBu)-PEG4-Asp(OtBu)-Rink Amide Resin

170.84 g m-PEG12-CH₂CH₂COOH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test.

### Step 3.3.6 Synthesis of NH₂-Lys(PEG12)-Asp(OtBu)-PEG4-Asp(OtBu)-Rink Amide Resin

2400 mL of de-Dde reagent was added, and the reaction was stirred at room temperature under nitrogen for 0.5 h, and then filtered. After repeating the operation 3 times, the resin was washed with DMF and DCM successively and showed blue color in the subsequent ninhydrin test.

### Step 3.3.7 Synthesis of Fmoc-Gly-Gly-Gly-Lys(PEG12)-Asp(OtBu)-PEG4-Asp(OtBu)-Rink Amide Resin

111.08 g Fmoc-Gly-Gly-Gly-OH and 48.64 g HOBT were weighed and dissolved in 2000 mL DMF and 80.0 mL DIC solution. After being placed in an ice bath at −10° C. for 0.5 h, it was slowly added to the reaction kettle with resin, and the reaction was stirred at room temperature for 2-4 h with nitrogen, and then filtered. The resin was washed with DMF and DCM successively and showed colorless or light yellow in the subsequent ninhydrin test. The resin peptide was washed three times with anhydrous ethanol, filtered and waited for cleavage.

### Step 3.3.8 Synthesis of Compound 3

10000 mL cleavage reagent (TFA:TIS:H₂O=95:2.5:2.5) was added to the 10L reactor and cooled to −10±2° C. The dried and weighed resin was added. The reaction was warmed to room temperature and stirred under nitrogen for 2-3 h. After that, the resin was filtered and washed once with 100 mL of TFA. The filtrate and washing solution were combined.

40 L of pre-cooled (below −10° C.) cold ether was added into product solution. The mixture was stirred for 10 minutes, and then the precipitate was centrifuged. The supernatant was discarded after centrifugation, and the precipitate was collected and washed with cold ether, and the precipitate was centrifuged again (Each time the centrifugation speed was set to 3600 rpm, the centrifugation time was 5 minutes, and the temperature of the centrifuge cavity was −5° C.).

The precipitate was purified by Prep-HPLC and lyophilized to afford Compound 3.

### 3.4 Synthesis of Compound 4

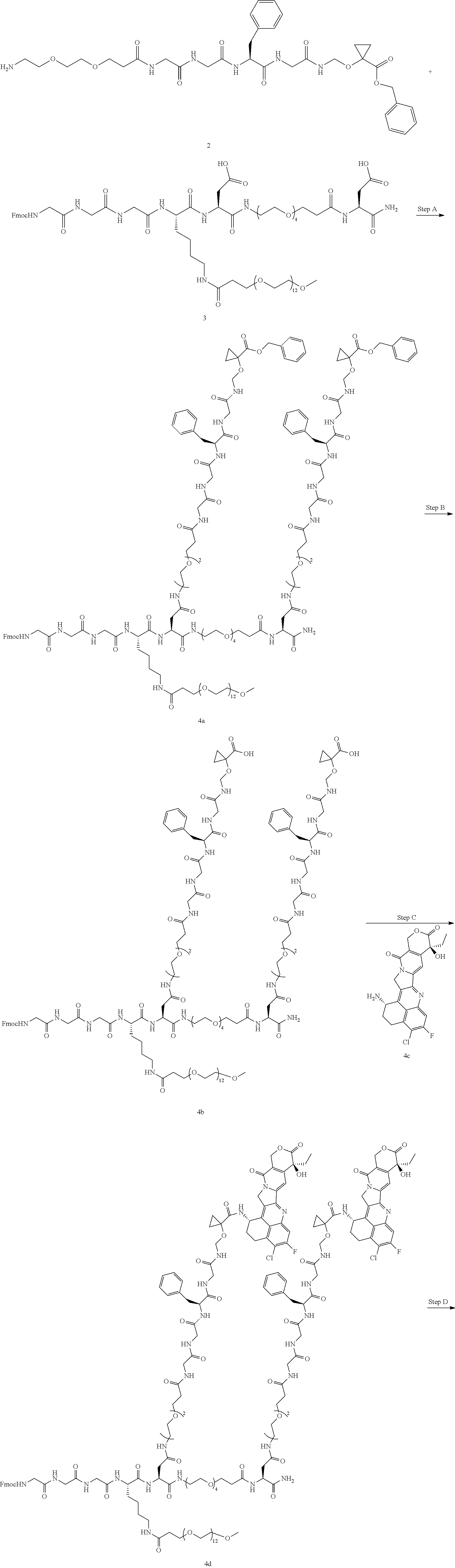

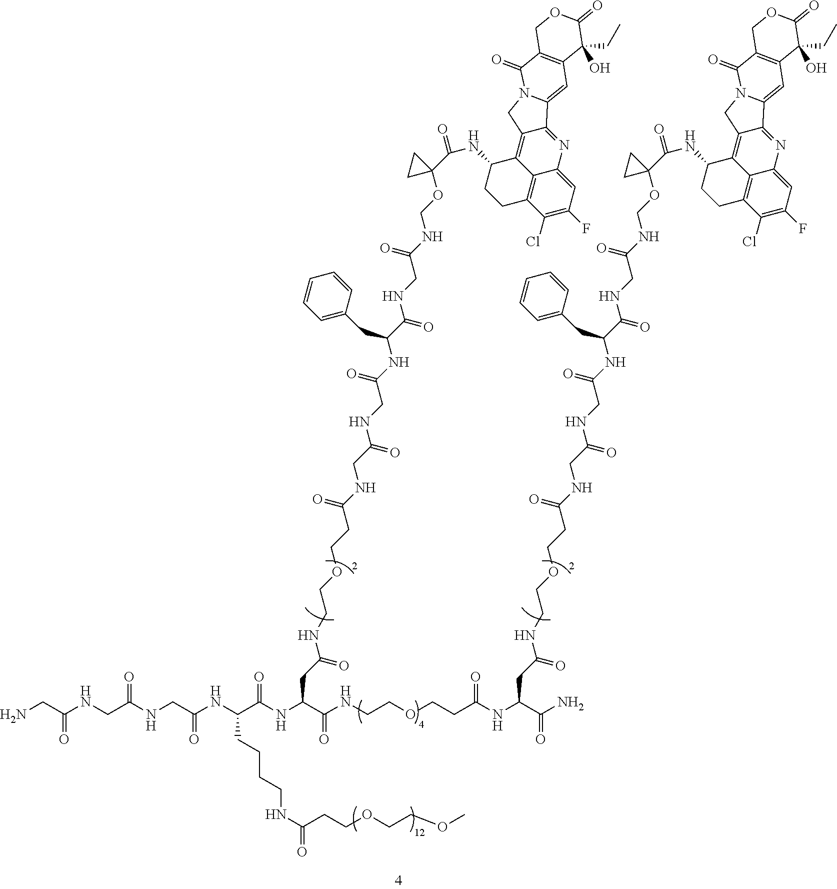

### Step A: Synthesis of Compound 4a

To a stirred solution of Compound 3 (400 mg, 0.245 mmol) and Compound 2 (377 mg, 0.539 mmol) in DMF (6 ml) was added DIPEA (159 mg, 1.23 mmol), followed by the addition of HATU (233 mg, 0.613 mmol), the resulting reaction mixture was stirred at room temperature for 2 h. Upon completion of the reaction monitored by HPLC, the reaction mixture was purified by prep-HPLC to yield Compound 4a as a white solid (380 mg, 52% yield). MS (ESI) calcd for C₁₄₂H₂₀₇O₄₉N₂₁ [(M+3H)/3]⁺: 997.8. Found: 875.9 (fragmented mass).

### Step B: Synthesis of Intermediate Compound 4b

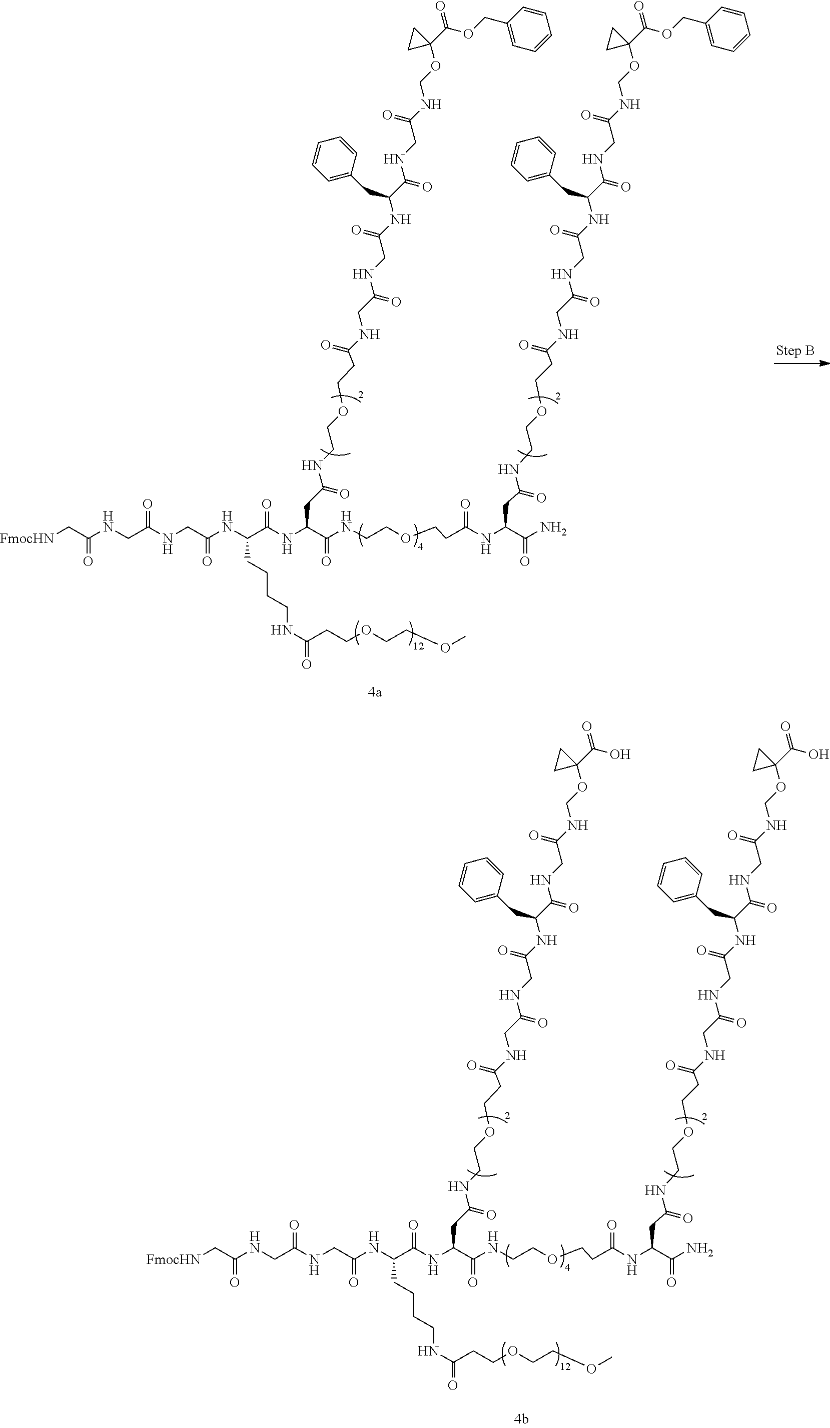

To a stirred solution of Compound 4a (380 mg, 0.245 mmol) in water (80 ml) was added palladium hydroxide on carbon (5 wt. % loading, 38 mg). The air in the system was replaced with hydrogen for 3 times, the resulting reaction mixture was stirred at room temperature. Upon completion, the reaction solution was filtered and purified by prep-HPLC to afford Compound 4b as a white solid (270 mg, 76% yield). MS (ESI) calcd for C₁₂₈H₁₉₅O₄₉N₂₁ [(M+3H)/3]⁺: 937.8. Found: 875.9 (fragmented mass).

### Step C: Synthesis of Intermediate Compound 4d

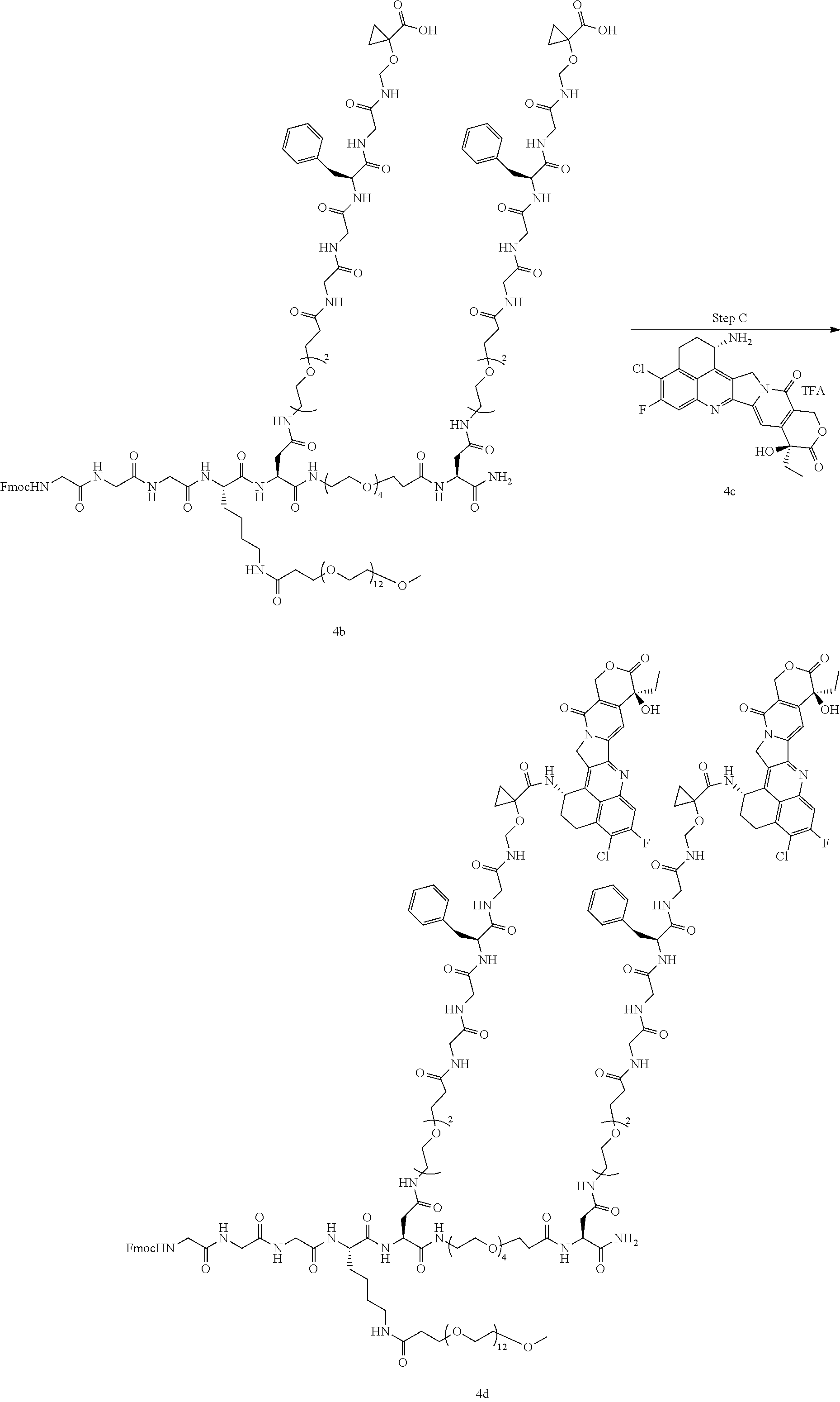

To the solution of Compound 4b (270 mg, 0.096 mmol) and Compound 4c (120 mg, 0.211 mmol) in DMF (5 ml) was added DIPEA (62 mg, 0.48 mmol) and HATU (92 mg, 0.24 mmol), the resulting reaction solution was allowed to stir at rt. After the completion of reaction monitored by HPLC, the reaction mixture was directly purified by prep-HPLC to give Compound 4d as a light yellow solid (235 mg, 66% yield). MS (ESI) calcd for C₁₇₄H₂₂₉O₅₅C₁₂F₂N₂₇ [(M+3H)/3]⁺: 1229.2. Found: 1229.3.

Synthesis of Compound 4c is showed below:

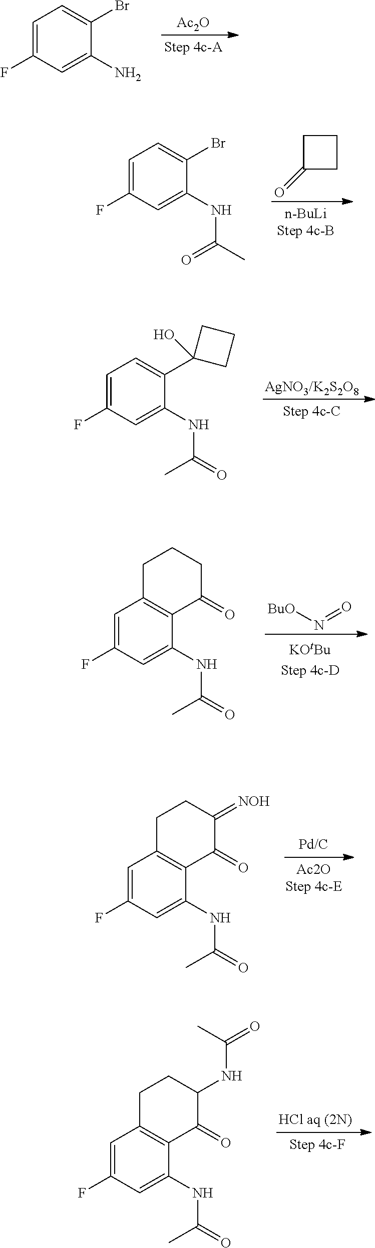

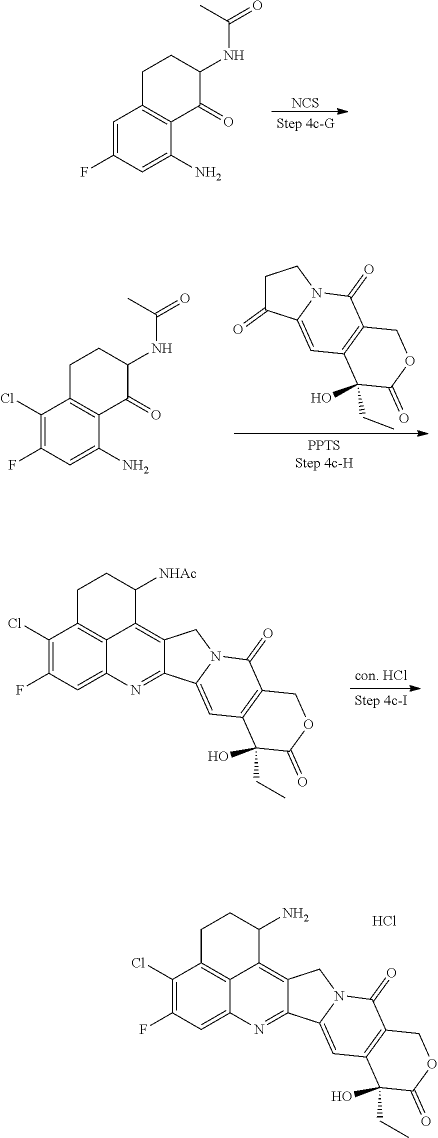

Step 4c-A: N-(2-bromo-5-fluorophenyl)acetamide: To a stirred solution of acetic anhydride (214 g, 2.10 mol) in acetic acid (500 mL) was added con. H₂SO₄ (3 mL), followed with 2-bromo-5-fluoroaniline (100 g, 526.27 mmol) in portions at room temperature. The mixture was stirred for 3 h, then poured into 2000 mL ice-water. A precipitate was formed, which was collected by filtration and dried in vacuo at room temperature to afford N-(2-bromo-5-fluorophenyl) acetamide (105 g) as a yellow solid. ¹H NMR (400 MHz, DMSO-d₆) δ 7.68 (dd, J=8.9, 6.0 Hz, 1H), 7.61 (ddd, J=10.7, 5.3, 3.1 Hz, 1H), 7.02 (ddd, J=8.9, 8.0, 3.1 Hz, 1H), 2.11 (s, 3H). MS m/z 232.0 (M+H).

Step 4c-B: N-(5-fluoro-2-(1-hydroxycyclobutyl)phenyl)acetamide: To a stirred solution of N-(2-bromo-5-fluorophenyl) acetamide (105 g, 452.48 mmol) in THF (1000 mL) was added n-BuLi (594 mL, 1.6 M in n-hexane, 950.22 mmol) dropwise over 1 h at −78° C. After completion, the mixture was stirred for 0.5 h under N₂. Then a solution of cyclobutanone (38.06 g, 542.98 mmol) in THF (50 mL) was added dropwise at −78° C. over 0.5 h, the mixture was stirred at −78° C. to room temperature for 6 h. The mixture was poured into 500 mL saturated NH₄Cl aq at 0° C. Extracted with ethyl acetate (500 mL×3), washed with brine (250 mL×2), dried over Na₂SO₄ and concentrated. The mixture was triturated with (PE/EA=1:1, 100 mL) for 10 mins, filtered and the cake was collected and dried in vacuo to afford N-(5-fluoro-2-(1-hydroxycyclobutyl)phenyl)acetamide (24 g) as a yellow solid. MS m/z 206.1 (M-18+H), 246.1 (M+Na).

Step 4c-C: N-(3-fluoro-8-oxo-5,6,7,8-tetrahydronaphthalen-1-yl)acetamide: To a stirred mixture of N-(5-fluoro-2-(1-hydroxycyclobutyl)phenyl) acetamide (24 g, 107.50 mmol) in CH₂Cl₂ (170 mL) and water (170 mL) was added silver nitrate (AgNO₃) (5.48 g, 32.25 mmol) and potassium persulfate (K₂S₂O₈) (58.12 g, 215.01 mmol), the mixture was stirred at 30° C. for 6 h. The mixture was filtered on Celite and washed with CH₂Cl₂ (100 mL), the filtrate was concentrated and purified by FCC (EA/PE=0-40%) to afford N-(3-fluoro-8-oxo-5,6,7,8-tetrahydronaphthalen-1-yl) acetamide (14 g) as a light yellow solid. MS m/z 222.1 (M+H).

Step 4c-D: N-(3-fluoro-7-(hydroxyimino)-8-oxo-5,6,7,8-tetrahydronaphthalen-1-yl)acetamide: To a stirring mixture of N-(3-fluoro-8-oxo-5,6,7,8-tetrahydronaphthalen-1-yl) acetamide (14 g, 63.28 mmol) in THF (500 mL) at 0° C. was added 1-butyl nitrite (8.48 g, 63.28 mmol), followed with t-BuOK (8.52 g, 75.94 mmol). The mixture was stirred at 0° C. for 2 h. After completion, the mixture was acidified by HCl (2 N) to adjust pH=3. The mixture was extracted by ethyl acetate (200 mL×3), washed by brine (100 mL×2), dried over Na₂SO₄ and concentrated under reduced pressure. The crude mixture was triturated with tert-butyl methyl ether (200 mL) for 10 mins, filtered and the cake was collected and dried in vacuo to afford N-(3-fluoro-7-(hydroxyimino)-8-oxo-5,6,7,8-tetrahydronaphthalen-1-yl) acetamide (12 g) as a yellow solid. MS m/z 251.1 (M+H).

Step 4c-E: N,N′-(3-fluoro-8-oxo-5,6,7,8-tetrahydronaphthalene-1,7-diyl)diacetamide: To a solution of N-(3-fluoro-7-(hydroxyimino)-8-oxo-5,6,7,8-tetrahydronaphthalen-1-yl)acetamide (12 g, 47.96 mmol) in acetic anhydride (90 mL) and THF (90 mL) was added 10% Pd/C (1 g), the mixture was stirred at 25° C. under H₂ atmosphere for 16 h. After cooling to 0° C., Et₃N (20 mL) was added dropwise, the mixture was stirred at 0° C. for 1 h. Filtered on Celite, the filtrate was poured into ice-water (500 mL). Extracted with ethyl acetate (500 mL×3), washed with brine (250 mL×2), dried over Na₂SO₄ and concentrated. The residue was triturated with tert-butyl methyl ether (120 mL) for 10 mins, filtered and the cake was collected and dried in vacuo to give N,N′-(3-fluoro-8-oxo-5,6,7,8-tetrahydronaphthalene-1,7-diyl)diacetamide (7.9 g) as a yellow solid. MS m/z 279.1 (M+H).

Step 4c-F: N,N′-(3-fluoro-8-oxo-5,6,7,8-tetrahydronaphthalene-1,7-diyl)diacetamide: To a solution of N,N′-(3-fluoro-8-oxo-5,6,7,8-tetrahydronaphthalene-1,7-diyl)diacetamide (7.9 g, 28.39 mmol) in MeOH (150 mL) was added HCl aq (2 N, 150 mL), the mixture was stirred at 50° C. for 7 h. After cooling to 0° C., Sat. NaHCO₃aq was added dropwise to adjust pH=8. Extracted with ethyl acetate (200 mL×3), washed with brine (200 mL×2), dried over Na₂SO₄ and concentrated under reduced pressure to give N,N′-(3-fluoro-8-oxo-5,6,7,8-tetrahydronaphthalene-1,7-diyl)diacetamide (6.0 g) as a yellow solid. ¹H NMR (400 MHz, Chloroform-d) δ 6.57 (s, 3H), 6.18 (td, J=11.1, 2.4 Hz, 2H), 4.52 (dt, J=13.3, 5.0 Hz, 1H), 3.13 (ddd, J=17.5, 13.0, 4.6 Hz, 1H), 3.00-2.81 (m, 1H), 2.69 (dtd, J=9.4, 4.6, 2.5 Hz, 1H), 2.09 (s, 3H), 1.79 (qd, J=13.0, 4.3 Hz, 1H). MS m/z 237.1(M+H).

Step 4c-G: N-(8-amino-5-chloro-6-fluoro-1-oxo-1,2,3,4-tetrahydronaphthalen-2-yl)acetamide: To a solution of N,N′-(3-fluoro-8-oxo-5,6,7,8-tetrahydronaphthalene-1,7-diyl)diacetamide (4.0 g, 16.93 mmol) in DMF (80 mL) was added NCS (2.26 g, 16.93 mmol) in portions at 0° C., the mixture was stirred at room temperature for 16 h. The mixture was poured into 200 mL ice-water. A precipitate was formed, which was collected by filtration and dried in vacuo at room temperature to afford N-(8-amino-5-chloro-6-fluoro-1-oxo-1,2,3,4-tetrahydronaphthalen-2-yl) acetamide (4.0 g) as a yellow solid. ¹H NMR (400 MHz, DMSO-d₆) δ 8.11 (d, J=8.0 Hz, 1H), 7.71 (s, 2H), 6.62 (d, J=11.9 Hz, 1H), 4.53 (ddd, J=13.0, 8.0, 4.7 Hz, 1H), 3.18-3.04 (m, 1H), 2.91 (ddd, J=17.5, 12.4, 4.8 Hz, 1H), 2.21-2.08 (m, 1H), 1.99-1.83 (m, 4H). MS m/z 271.0(M+H).

Step 4c-H: N-(9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)acetamide: To a mixture of N-(8-amino-5-chloro-6-fluoro-1-oxo-1,2,3,4-tetrahydronaphthalen-2-yl)acetamide (4.0 g, 14.78 mmol) in toluene (400 mL) was added(S)-4-ethyl-4-hydroxy-7,8-dihydro-1H-pyrano[3,4-f]indolizine-3,6,10 (4H)-trione (4.28 g, 16.25 mmol), pyridinium p-Toluenesulfonate (1.11 g, 4.43 mmol) and o-cresol (10 mL), the mixture was heated to reflux under N₂ for 24 h. The solvent was removed by reduced pressure and the mixture was purified by FCC (THF/CH₂Cl₂=0-60%) to afford N-(9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)acetamide (4.1 g) as a brown solid. MS m/z 498.1(M+H).

Step 4c-I: (9S)-1-amino-4-chloro-9-ethyl-5-fluoro-9-hydroxy-1,2,3,9,12,15-hexahydro-10H,13H-benzo[de]pyrano[3′,4′: 6,7]indolizino[1,2-b]quinoline-10,13-dione: A mixture of N-(9S)-4-chloro-9-ethyl-5-fluoro-9-hydroxy-10,13-dioxo-2,3,9,10,13,15-hexahydro-1H,12H-benzo[de]pyrano[3′,4′: 6,7]indolizino[1,2-b]quinolin-1-yl)acetamide (2.0 g, 4.02 mmol) in 20 mL con. HCl aq was stirred at 70° C. under N₂ for 36 h. The mixture was concentrated under reduced pressure to give crude (9S)-1-amino-4-chloro-9-ethyl-5-fluoro-9-hydroxy-1,2,3,9,12,15-hexahydro-10H,13H-benzo[de]pyrano[3′,4′: 6,7]indolizino[1,2-b]quinoline-10,13-dione hydrochloride (2 g) as a brown solid. MS (ESI) m/z 456.1 (M+H).

### Step 4c-J: Preparation of Intermediate Salt Form of Compound 4c

Compound 4c and Compound 4c′ were prepared by prep-HPLC from (9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinoline-10,13-dione hydrochloride as TFA salt.

| | | | | Retention |
|---|---|---|---|---|
| | | | | time on |
| | | | MS | HPLC |
| Number | Structure | ¹HNMR | (M + H) | (min) |
| 4c | 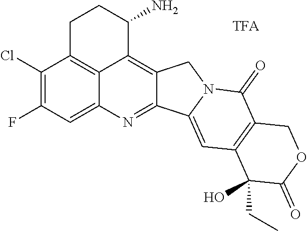 [SMILES: CC[C@@]1(O)C(=O)OCc2c1cc1n(c2=O)Cc2c-1nc1cc(F)c(Cl)c3c1c2[C@@H](N)CC3 ; "TFA"] | ¹H NMR (400 MHz, DMSO-d6) δ8.51 (d, J = 4.8 Hz, 3H), 8.17 (d, J = 10.2 Hz, 1H), 7.38 (s, 1H), 6.56 (s, 1H), 5.74 (d, J = 19.4 Hz, 1H), 5.52-5.40 (m, 3H), 5.16 (s, 1H), 3.44 (dd, J = 16.3, 4.1 Hz, 1H), 3.19 (t, J = 13.9 Hz, 1H), 2.57 (d, J = 14.0 Hz, 1H), 2.26 (t, J = 14.3 Hz, 1H), 1.89 (hept, J = 7.0 Hz, 2H), 0.89 (t, J = 7.3 Hz, 3H). | 456.0 | 1.395 |
| 4c′ | 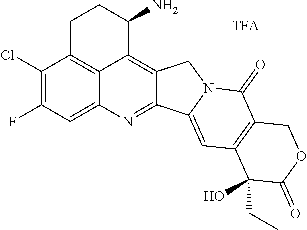 [SMILES: CC[C@@]1(O)C(=O)OCc2c1cc1n(c2=O)Cc2c-1nc1cc(F)c(Cl)c3c1c2[C@H](N)CC3 ; "TFA"] | ¹H NMR (400 MHz, DMSO-d6) δ 8.56 (s, 2H), 8.17 (d, J = 10.1 Hz, 1H), 7.38 (s, 1H), 6.57 (s, 1H), 5.74 (d, J = 19.4 Hz, 1H), 5.53-5.28 (m, 3H), 5.16 (s, 1H), 3.59-3.01 (m, 2H), 2.35-2.19 (m, 2H), 2.00-1.78 (m, 2H), 0.88 (t, J = 7.3 Hz, 3H). | 456.0 | 1.541 |

Conditions of HPLC above: Equipment: Agilent 1200; Chromatographic column: Waters XBridge C18 4.6*50 mm, 3.5 um; Flow:2.0 mL/min; Gradient elute: 5.0%-95.0%-95.0%-5.0%-5.0%, 0.00 min-1.50 min-2.50 min-2.52 min-3.00 min; Temperature: 40° C.; Phase: A: Acetonitrile, B: H₂O (0.05% TFA); Wavelength: 214 nm/254 nm.

### Step D: Synthesis of Compound 4 (H0058)

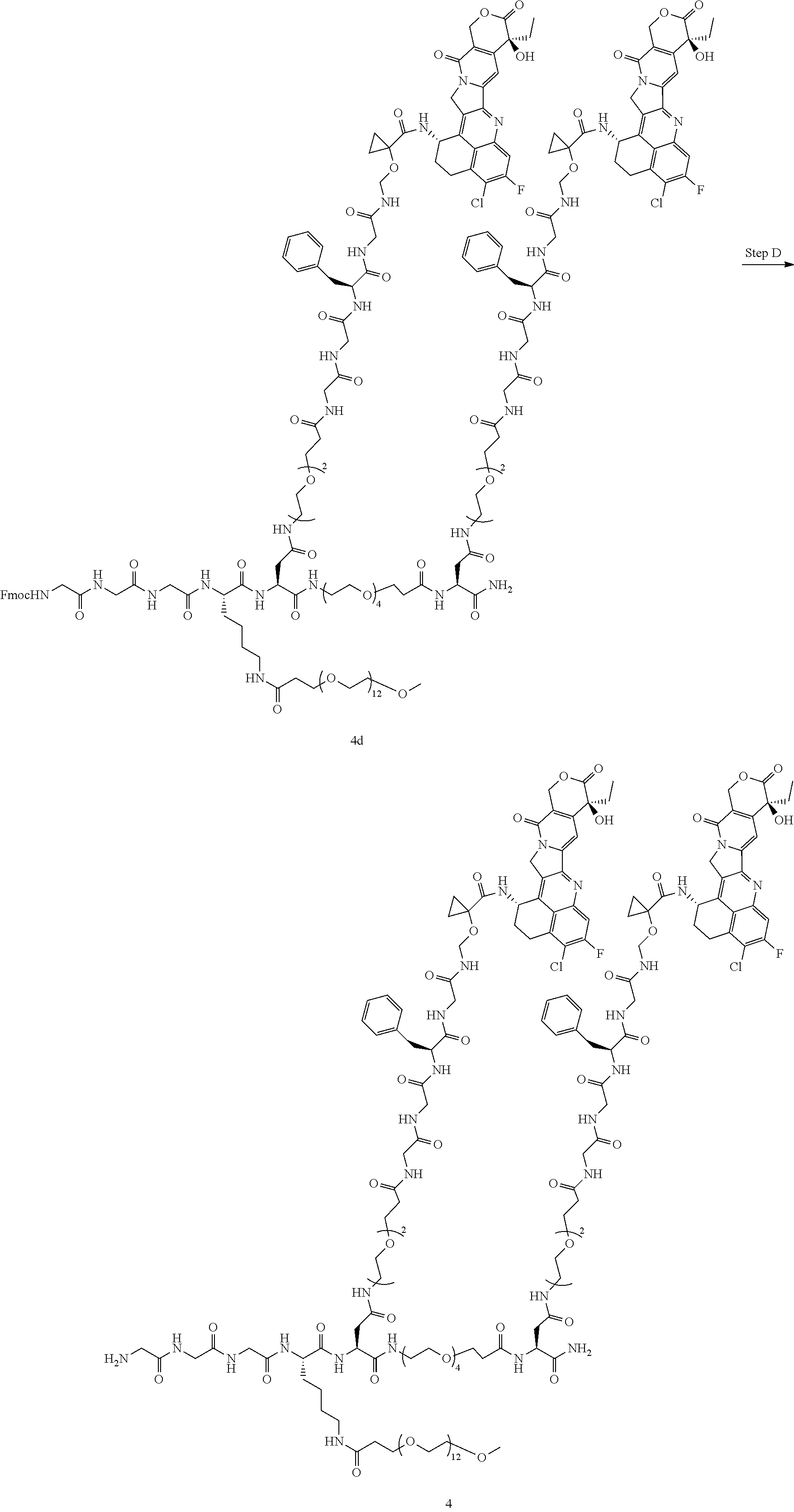

To the solution of Compound 4d (210 mg, 0.057 mmol) in DMF (5 ml) was added diethylamine (0.5 ml), the resulting mixture was stirred at rt for 15 min, the system was adjusted to neutrality with 10% TFA aqueous solution at 0° C., then the reaction mixture was purified by prep-HPLC and lyophilized to afford Compound 4 as a light yellow solid (145 mg, 73% yield). MS (ESI) calcd for C₁₅₉H₂₁₉O₅₃Cl₂F₂N₂₇ [(M+3H)/3]⁺: 1155.2. Found: 1155.3.

### 3.5 Synthesis of Compound 5

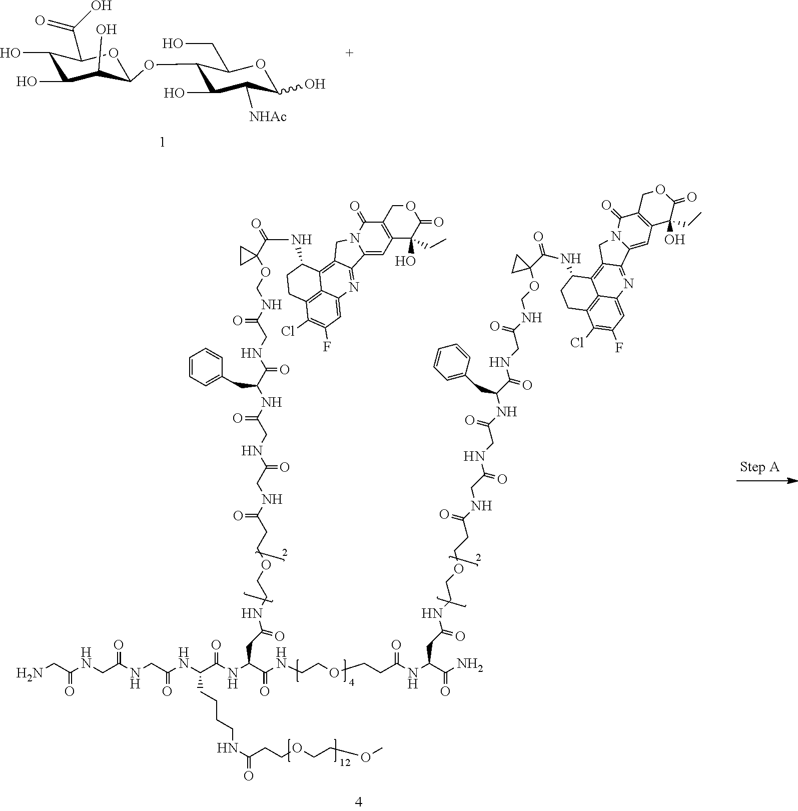

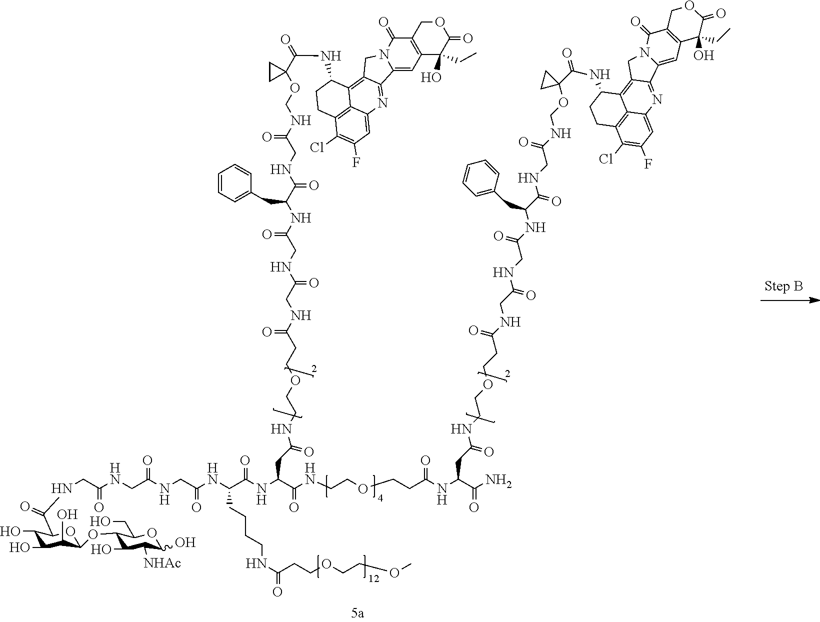

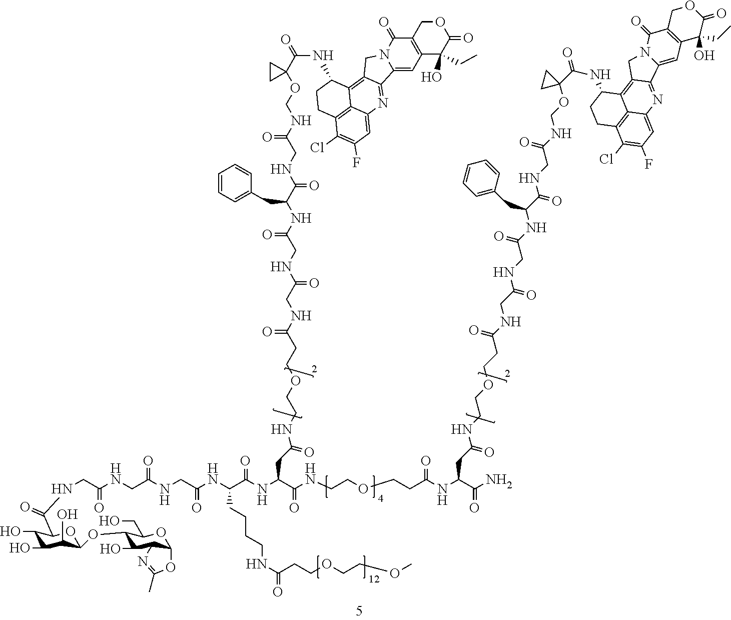

Step A: To a stirred solution of Compound 1 and Compound 4 were added DIEA and HATU, the resulting mixture was stirred at a certain temperature (e.g., −20° C. to 40° C.), upon completion, the reaction was directly purified by prep-HPLC to afford Compound 5a as a light yellow solid (337 mg, 63% yield). MS (ESI) calcd for C₁₇₃H₂₄₃C₁₂F₂N₂₈O₆₄ [(M+3H)/3]⁺: 1281.5. Found: 1281.7.

Step B: To a stirred solution of Compound 5a in water was added Et₃N and 2-Chloro-1,3-dimethylimidazolidinium chloride (DMC, CAS: 37091-73-9), the resulting mixture was stirred at certain temperature (e.g., −20° C. to 40° C.), after the completion of the reaction monitored by HPLC, the reaction mixture was directly purified by prep-HPLC to afford Compound 5 as a light yellow solid (130 mg, 74% yield). MS (ESI) calcd for C₁₇₃H₂₄₃C₁₂F₂N₂₈O₆₄ [(M+H₂O+3H)/3]⁺: 1281.5. Found: 1281.7.

### 3.6 Synthesis of Compound 7

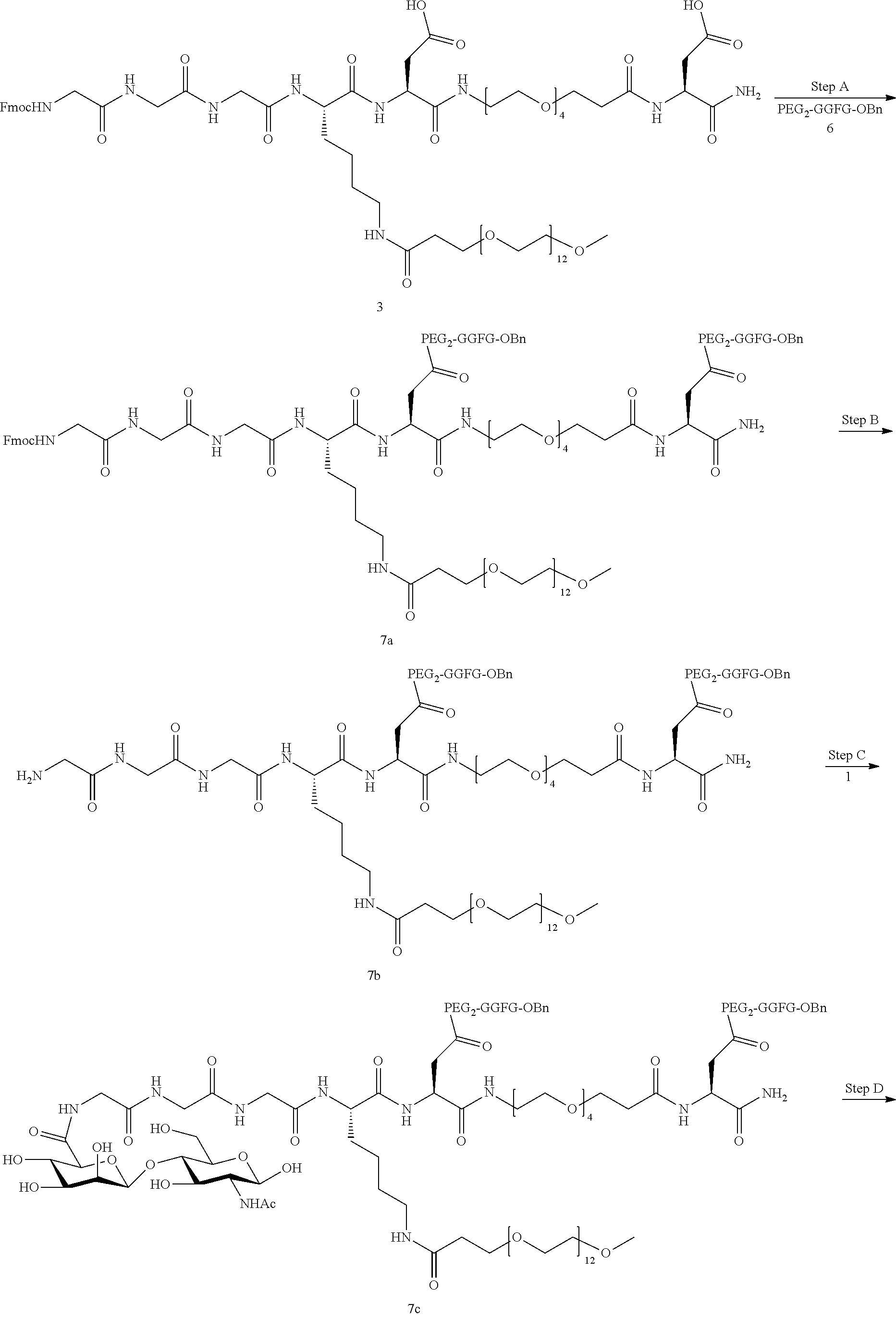

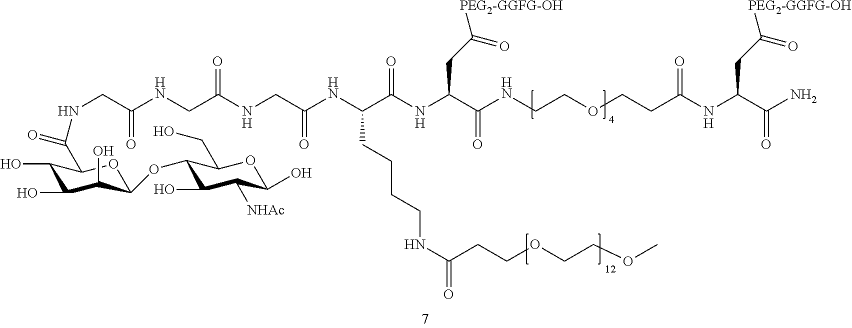

### Step A: Synthesis of Compound 7a

To a stirred solution of Compound 3 and Compound 6 (PEG2-GGFG-OBn) in DMF was added DIEA and HATU, the resulting reaction mixture was stirred at rt. Upon completion, the reaction mixture was directly used in the next step without any purification.

### Step B: Synthesis of Compound 7b

To the reaction mixture of last step was added Et₂NH, the resulting mixture was allowed to stir for 15 min at rt, and then neutralized with 30% HOAc, directly purified by prep-HPLC to afford Compound 7b as a white solid (560 mg, 62% yield over 2 steps). MS (ESI) calcd for C₁₁₇H₁₈₃N₁₉O₄₃ [(M+2H)/2]⁺: 1272.1. Found: 1272.2.

### Step C: Synthesis of Compound 7c

To a stirred solution of Compound 7b and Compound 1 in DMF was added DIEA and HATU, the resulting reaction mixture was stirred at rt, upon completion, the reaction mixture was purified by prep-HPLC to afford Compound 7c as a white solid (400 mg, 87% yield). MS calcd for C₁₃₁H₂₀₄N₂₀O₅₄ [(M+3H+H₂O)/3]⁺: 980.8. Found: 981.0.

### Step D: Synthesis of Compound 7

To a stirred solution of Compound 7c (400 mg) in water was added palladium hydroxide on carbon (5 wt. % loading). The air in the system was replaced with hydrogen for 3 times, the resulting reaction mixture was stirred at room temperature. Upon completion, the reaction solution was filtered and purified by prep-HPLC to afford Compound 7 as a white solid (240 mg, 64% yield). MS (ESI) calcd for C₁₁₇H₁₉₂N₂₀O₅₄ [(M+2H)/2]⁺: 1372.1. Found: 1372.29.

### 3.7 Synthesis of Compound 8

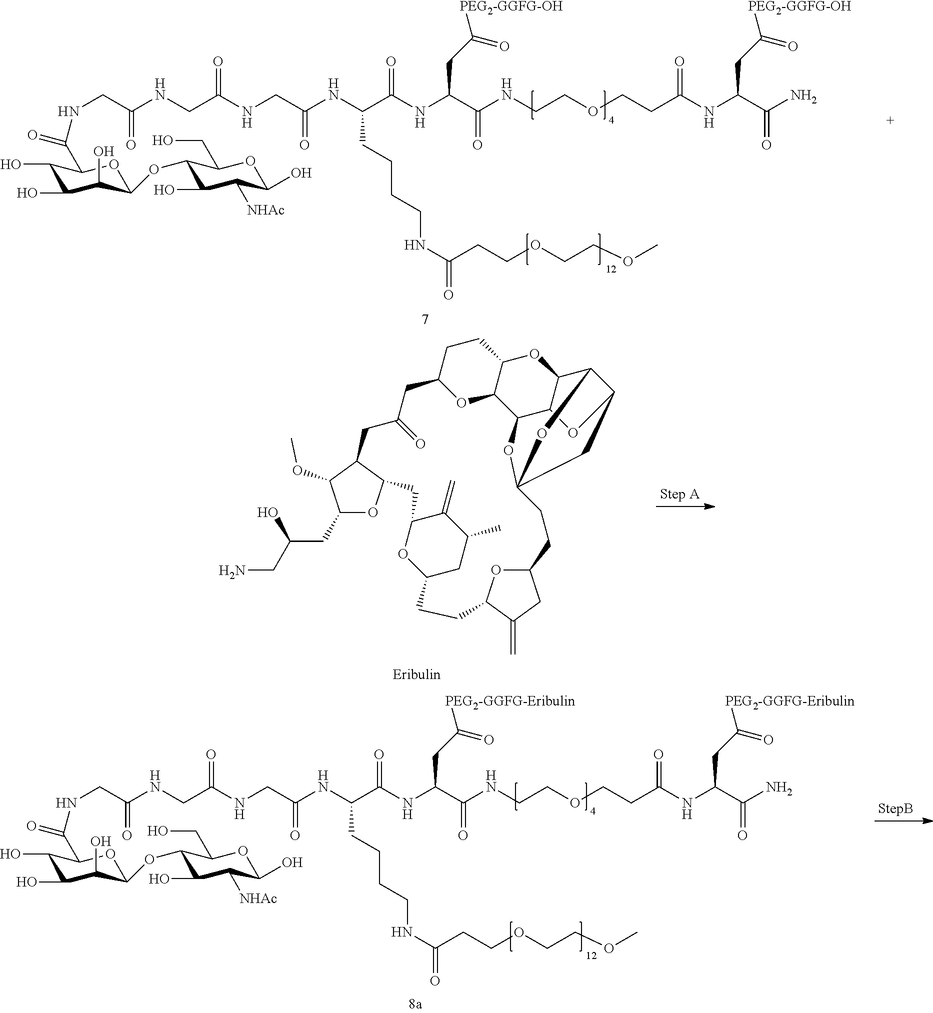

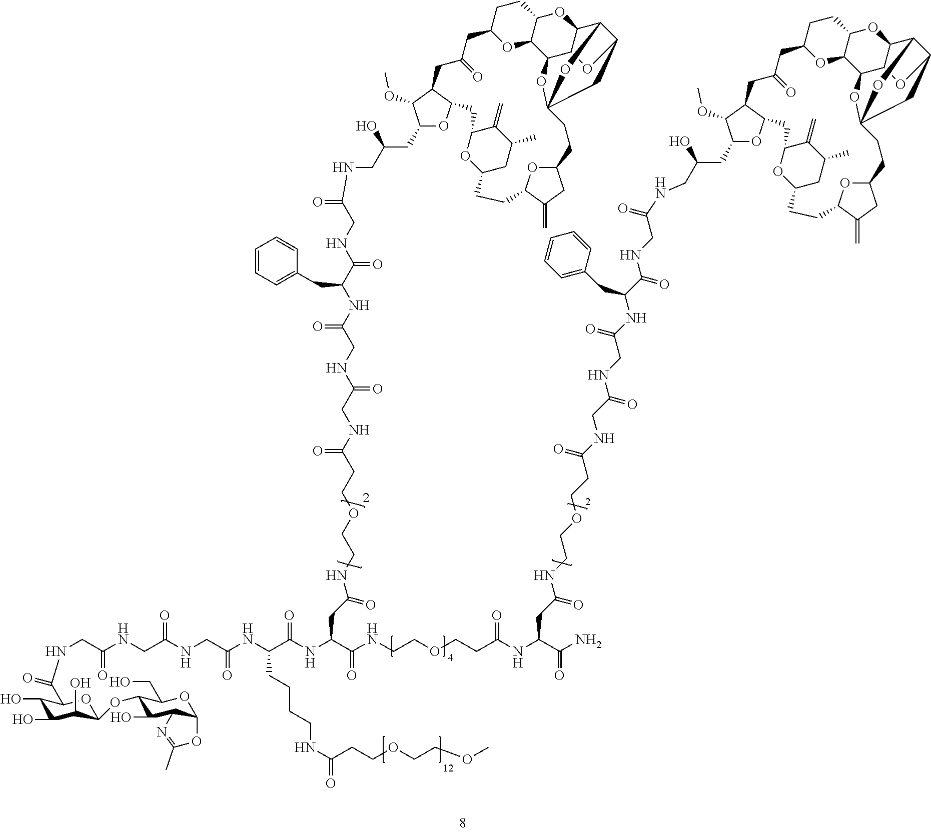

### Step A: Synthesis of Compound 8a

To a stirred solution of Compound 7 and Eribulin in DMF were added DIEA and HATU, the resulting reaction mixture was stirred at rt. Upon completion, the reaction mixture was purified by prep-HPLC to yield Compound 8a as a white solid (245 mg, 67% yield). MS (ESI) calcd for C₁₉₇H₃₀₆N₂₂O₇₄ [(M+3H)/3]⁺: 1389.0. Found: 1389.1.

### Step B: Synthesis of Compound 8

To a stirred solution of Compound 8a in water was added Et₃N and 2-Chloro-1,3-dimethylimidazolidinium chloride (DMC, CAS: 37091-73-9), the resulting mixture was stirred at certain temperature (e.g., −20° C. to 40° C.), after the completion of the reaction monitored by HPLC, the reaction mixture was directly purified by prep-HPLC to afford Compound 8 as a white solid (139 mg, 63% yield). MS (ESI) calcd for C₁₉₇H₃₀₄N₂₂O₇₃ [(M+3H+H₂O)/3]⁺: 1389.0. Found: 1389.1.

### 3.8 Synthesis of Compound 9, Linker-Payload for Ligase

The preparation of Compound 9 (linker 2-DM1 intermediate (n=3, ring-open)) is referenced to the Example 2 of US20170112944 A1, and the entirety is incoraported herein. The structure of linker 2-DM1 intermediate is as shown below:

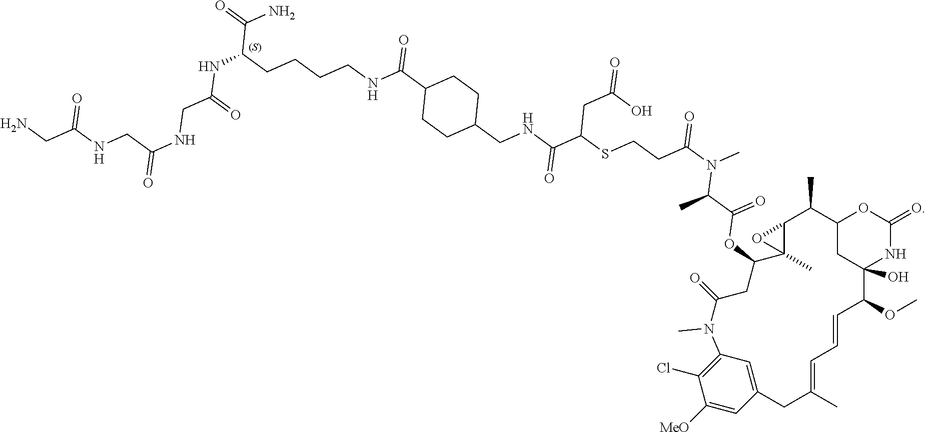

### 3.9 Synthesis of Compound 10, Linker-Payload for Ligase

Compound 10 (MS (ESI) calcd for C₉₄H₁₄₈N₁₆O₂₉ [(M+2H)/2]⁺: 983.53. Found: 983.93) was synthesized by a general method using the intermediates of formula (V′) and formula (A).

### Example 4 Preparation of Antibodies

Production, purification and characterization of anti-human ErbB2/Her2 antibody T-LCCT_{L}-HC are referenced to the Example 1 of US20170112944 A1. The production and purification of anti-human ErbB3/Her3 antibody refer to the method of the the Example 1 of US20170112944 A1 by using the anti-human ErbB3/Her3 antibody (SEQ ID NO: 33 and SEQ ID NO: 34) to replace the anti-human ErbB2/Her2 antibody T-LCCT_{L}-HC (SEQ ID NO: 31 and SEQ ID NO: 32).

### Example 5 Preparation of Antibody-Drug Conjugates with Dual-Enzyme Catalyzed Site-Specific Conjugation

The present disclosure formed ADC drugs by specific recognition and cleavage of the Fc glycans located at Asn-297 of the antibody followed by the site-specific conjugation of linker-payload to the Asn-297 glycan residual mediated by immobilized endoglycosidase.

### 5.1 Preparation and Analysis of ADC-1

### 5.1.1 Solid-phase Preparation of ADC Drug ADC-1

Preparation of ADC drug ADC-1 was based on the glycan-remodeling conjugation of the antibody with linker-payload mediated by the immobilized endoglycosidase catalysis. In 1× endoglycosidase buffer, the antibody was mixed thoroughly with linker-payload (Compound 5) in an appropriate molar ratio (1:1 to 1:100), then the immobilized endoglycosidase medium was added and mixed. The conjugation reaction in the mixed suspension was carried out at certain temperature (4-40° C.) for a certain time (0.5-20 h). Upon completion, the solid phase conjugation mixture was centrifuged, purified, ultrafiltered or dialyzed to remove unreacted drug intermediates. Purified ADC drug ADC-1 was stored in 1×PBS pH 7.4 at 2-8° C. or −80° C.

### 5.1.2 Analysis of ADC-1 by SDS-PAGE

After the conjugation, the purity and conjugation efficiency of ADC-1 were detected by SDS-PAGE. The result of SDS-PAGE of ADC-1 is shown in FIG. 4. The conjugation occurred on the heavy chain of the antibody, and the heavy chain of ADC-1 conjugated with linker-payload shows a significant molecular weight increase compared with the heavy chain of the monoclonal antibody with the sugar chain excision, demonstrating that linker-payload is successfully site-specific conjugated to the heavy chain of the mAb. Basically, no unconjugated linker-payload antibodies are observed in the conjugated products, the conjugation efficiency is over 95%, and the purity of the conjugated products are consistent with expectation.

### 5.1.3 Analysis of ADC-1 by HIC-HPLC

Proteomix HIC Butyl-NP5 4.6*100 mm 5 μm Non-Porous column (manufacturer: Sefen, PN: 431NP5-4610); 1.5M ammonium sulfate +20 mM phosphate buffer salt (pH 7.0), as mobile phase A; 20 mM phosphate buffer salt (pH 7.0):isopropanol=7:3 (v/v), as mobile phase B; flow rate 0.8 mL/min; gradient method: phase B was increased from 10% to 100% in 8 min; and 280 nm as the detection wavelength were used to detect the DAR distribution of the ADC drug ADC-1.

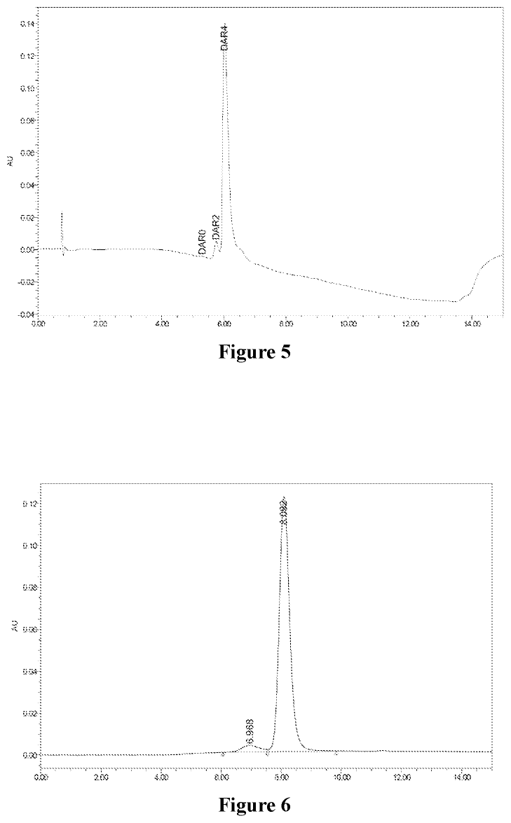

The assay results are shown in FIG. 5, where the unconjugated antibody is <5%; the DAR of conjugated product is predominantly to be 4, with an overall DAR value of about 3.9 for the ADC drug.

### 5.1.4 Analysis of ADC-1 by SEC-HPLC

TSKgel G3000SW_{XL} 7.8 mm I.D.*30 cm, 5 μm column (manufacturer: DongCao, PN: 0008541); 2*PBS:methanol=9:1 (v/v) as mobile phase; room temperature; isocratic; flow rate 1.0 mL/min; running time 15 min; 280 nm as the detection wavelength were used to analyze and detect the degree of high molecular weight aggregation of the ADC drug.

The results of the assay are shown in FIG. 6. The high molecular weight aggregation in the ADC drug is less than 5%, and the ADC sample at 8.1 min is mainly in the form of monomer.

### 5.2 Preparation and Analysis of ADC-2

### 5.2.1 Preparation of the ADC-2

Refer to the general preparation protocol described in Example 5.1 of the present disclosure (i.e., ADC-1) to furnish ADC-2, except that compound 8 is used as the linker-payload.

### 5.2.2 Analysis of ADC-2 by HIC-HPLC

Proteomix HIC Butyl-NP5 4.6*100 mm 5 μm Non-Porous column (manufacturer: Sefen, PN: 431NP5-4610); 1.5M ammonium sulfate +20 mM phosphate buffer salt (pH 7.0), pH 7.0 as mobile phase A; 20 mM phosphate buffer salt (pH 7.0):isopropanol=7:3 (v/v), as mobile phase B; flow rate 0.8 mL/min; gradient method: phase B was increased from 10% to 85% in 8 min; and 280 nm as the detection wavelength were used to detect the DAR distribution of the ADC drug ADC-2.

The assay results are shown in FIG. 7, where the unconjugated cytotoxic antibody is <5%; the DAR of conjugated product is predominantly to be 4, with an overall DAR value of about 3.52 for the ADC drug.

### 5.2.3 Analysis of ADC-2 by SEC-HPLC

TSKgel G3000SW_{XL} 7.8 mm I.D.*30 cm, 5 μm column (manufacturer: DongCao, PN: 0008541); 2*PBS:methanol=9:1 (v/v) as mobile phase; room temperature; isocratic; flow rate 1.0 mL/min; running time 15 min; 280 nm as the detection wavelength were used to analyze and detect the degree of high molecular weight aggregation of the ADC drug.

The results of the assay are shown in FIG. 8. The high molecular weight aggregation in the ADC drug is less than 6%, and the ADC sample at 7.87 min is mainly in the form of monomer.

### 5.3 Preparation and Analysis of Ligase-Catalyzed Antibody-Drug Conjugete ADC-3

The preparation and analysis of ligase-catalyzed antibody-drug conjugate are referenced to the Example 3-7 of US20170112944 A1.

The present disclosure conjugated the linker 2-DM1 intermediate (n=3, ring open) to the ErbB2/Her2 antibody ErbB2/Her2 T-LCCT_{L}-HC in a site-specific manner by transpeptidase to form the ADC drugs as shown below:

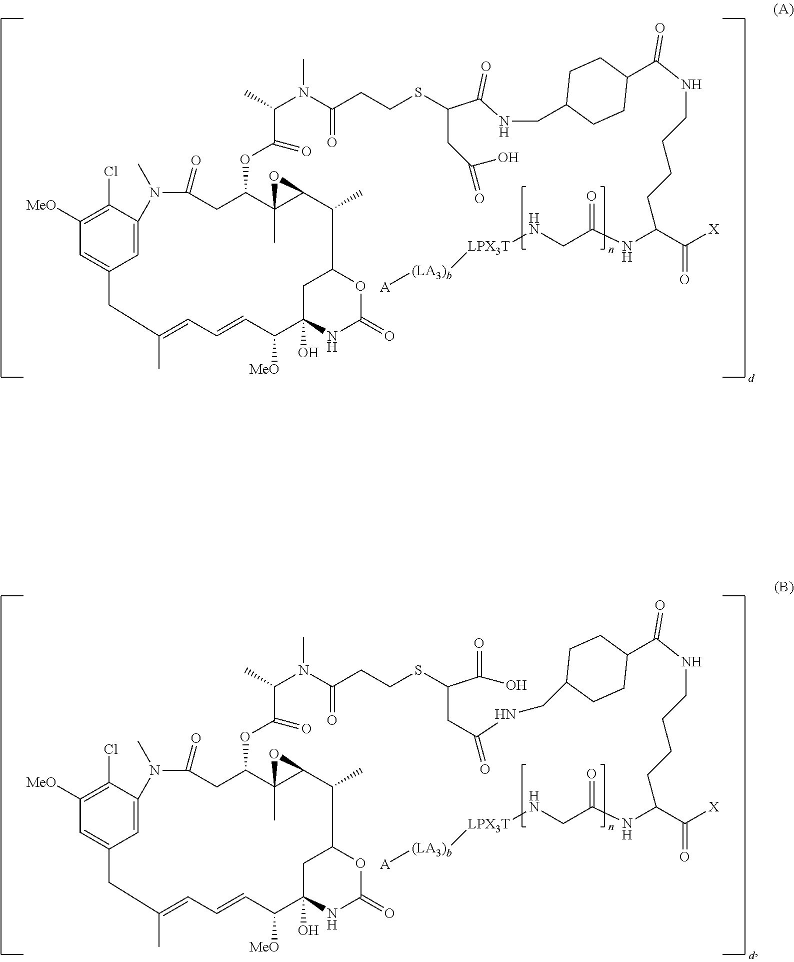

wherein n is 3, d is 2, and X in the ligase recognition sequence LPXT is glutamic acid (E).

### 5.4 Preparation and Analysis of Antibody-Drug Conjugate dpADC-1 with Dual-Enzyme Catalyzed Site-Specific Conjugation

The present disclosure was catalyzed by two enzymes acting separately to specifically conjugate in two different sites to form a unique dual-payload ADC drug (DAR2+DAR4).

The immobilized transpeptidase specifically recognizes and cleaves the light chain (LC) C-terminal enzyme recognition peptide sequence LPXTGG of antibody and then site-specifically connects the modified peptide sequence on the mAb with the linker-payload Compound 9 to form the ADC intermediate.

The immobilized endoglycosidase specifically recognizes and cleaves Fc glycans located at Asn-297 position of the antibody (heavy chain), and then connects the glycan-remodeled mAb with the linker-payload (Compound 5) to form the ADC intermediate.

Changing the sample solution:

ErbB2/Her2 antibodies were processed using ultrafiltration or dialysis or desalination columns, replacing their storage buffer with 50 mM Tris-HCl (pH between 5-8) and 150 mM NaCl.

### 5.4.1 One-Step Method

The ADC drug dpADC-1 was prepared based on the immobilized transpeptidases Sortase and endoglycosidase, which catalyzed the conjugation reaction of antibodies with two linker-payloads (Compound 9 and Compound 5). In 1×buffer, the antibody was mixed sufficiently with two linker-payloads (Compound 9 and Compound 5) in an appropriate molar ratio (1:1 to 1:100), respectively, and immobilized transpeptidase medium and endoglycosidase medium were added and mixed. Two solid phase enzyme media with immobilized transpeptidases and endoglycosidases on the substrate catalyzed the conjugation of the ErbB2/Her2 antibody with two linker-payloads (Compound 9 and Compound 5), respectively. The conjugation reaction in the mixed state was carried out at 4-40° C. for 0.5-20 h. Upon completion of the reaction, the solid phase reaction mixture was centrifuged, purified, ultrafiltered or dialyzed to remove unreacted drug intermediates. Purified ADC drug dpADC-1 was stored in 1×PBS pH 7.4 stored at 2-8° C. or −80° C.

### 5.4.2 Two-Step Method

In the first step, the conjugation reaction of the ErbB2/Her2 antibody with linker-payload (Compound 9) was first catalyzed using transpeptidase to prepare the conjugated drug ADC-3. In 1× transpeptidase buffer, the antibody was mixed sufficiently with linker-payload (Compound 9) in an appropriate molar ratio (1:1 to 1:100), and immobilized transpeptidase medium was added and mixed. The solid-phase transpeptidase medium had immobilized transpeptidase on a substrate that catalyzed the conjugation reaction of the ErbB2/Her2 antibody with linker-payload (Compound 9). The conjugation reaction in the mixed state was carried out at 4-40° C. for 0.5-20 h. Upon completion of the reaction, the solid phase conjugation reaction mixture was centrifuged and removed to obtain ADC-3.

In the second step, endoglycosidase was used to catalyze the conjugation reaction of ADC-3 (DAR2) with linker-payload (Compound 5) to prepare the conjugated drug dpADC-1. In 1×endoglycosidase buffer, ADC-3 and linker-payload (Compound 5) were mixed sufficiently in an appropriate molar ratio (1:1 to 1:100), and immobilized endoglycosidase medium was added and mixed. Solid-phase endoglycosidase media with immobilized endoglycosidase on a substrate catalyzed the conjugation reaction of ADC-3 with linker-payload (Compound 5). The conjugation reaction in the mixed state was carried out at 4-40° C. for 0.5-20 h. Upon completion of the reaction, the solid phase conjugation reaction mixture was centrifuged and removed to obtain ADC drug dpADC-1.

There is no difference between the first and second steps in order. The second step can be completed before proceeding to the first step.

The dpADC-1 was purified, ultrafiltered or dialyzed to remove unreacted drug intermediates. The purified ADC drug dpADC-1 was stored in 1×PBS pH 7.4 stored at 2-8° C. or −80° C.

### 5.4.3 Analysis of dpADC-1 (One-Step and Two-Step Method) by SDS-PAGE

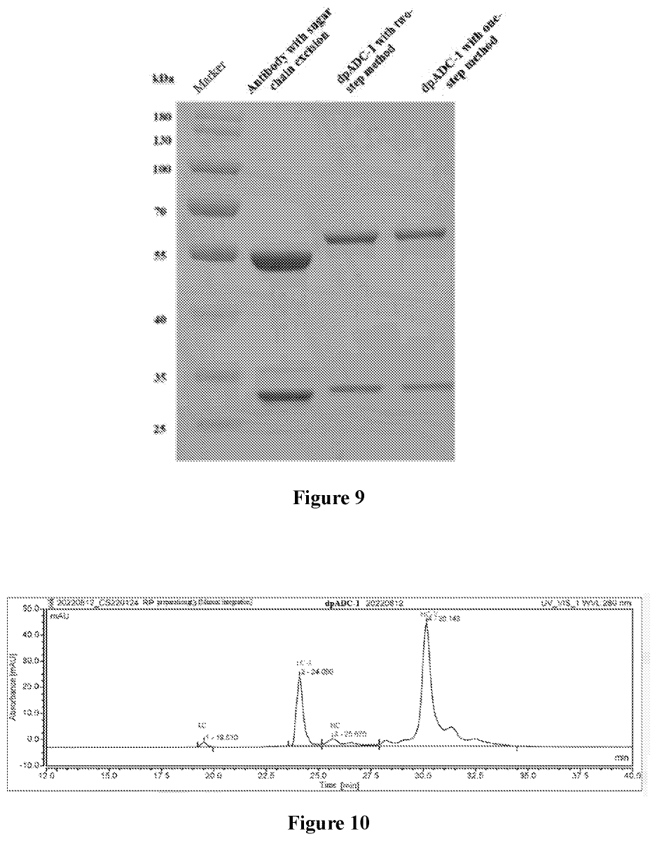

After the conjugation reaction, the purity and conjugation efficiency of dpADC-1 were detected by SDS-PAGE. The results of SDS-PAGE of dpADC-1 (one-step and two-step methods) are shown in FIG. 9. The conjugation reaction site-specific conjugation occurs on the light chain (transpeptidase conjugation) and heavy chain (endoglycosidase conjugation) of the antibody, and the light chain of dpADC-1 conjugated on linker-payload has a significant molecular weight transition compared with the monoclonal antibody light chain, demonstrating the successful targeted conjugation of linker-payload to the monoclonal antibody light chain molecule, and a significant molecular weight tansition of the dpADC-1 heavy chain conjugated on linker-payload compared with the monoclonal antibody heavy chain with the sugar chain excision, demonstrating the successful targeted conjugation of linker-payload to the monoclonal antibody heavy chain molecule. Basically, no unconjugated linker-payload antibodies are observed in the conjugated products, the conjugation efficiency is over 95%, and the purity of the conjugated products is in consistent with expectation. In addition, there is no difference in the conjugation efficiency between the one-step and two-step methods, which proved the good bio-orthogonality and could realize the “one-step” two-enzyme orthogonal conjugation, which greatly reduces the operation steps, significantly reduces the cost and realizes the homogeneous and controllable quality of CMC.

### 5.4.4 Analysis of ADC Drug dpADC-1 by RP-HPLC

XBridge® Protein BEH C4 Column, 300A, 3.5 μm, 4.6 mm*150 mm column (manufacturer: Waters, PN: 186004504) was used; 0.1% HCOOH and 0.025% TFA in Waters as mobile phase A; 0.1% HCOOH and 0.025% TFA in ACN as mobile phase B; flow rate of 0.4 mL/min; column temperature of 80° C.; gradient method: phase B was increased from 25% to 35% in 38 min; 280 nm as the detection wavelength were used to analyze the DAR distribution of ADC drug dpADC-1.

The detection results are shown in FIG. 10, the sum of the relative peak areas of the unconjugated cytotoxic antibody heavy and light chains is <7%; the majority of the conjugated product is product with a DAR of 2+4, and the overall DAR value of the ADC drug is about 5.62 (LC+HC=1.89+3.73).

### 5.4.5 Analysis of ADC Drug dpADC-1 by SEC-HPLC

TSKgel G3000SW_{XL} 7.8 mm I.D.*30 cm, 5 μm column (manufacturer: DongCao, PN: 0008541); 2*PBS:methanol=9:1 (v/v) as mobile phase; room temperature; isocratic; flow rate 1.0 mL/min; running time 15 min; 280 nm as the detection wavelength were used to analyze and detect the degree of high molecular weight aggregation of the ADC drug.

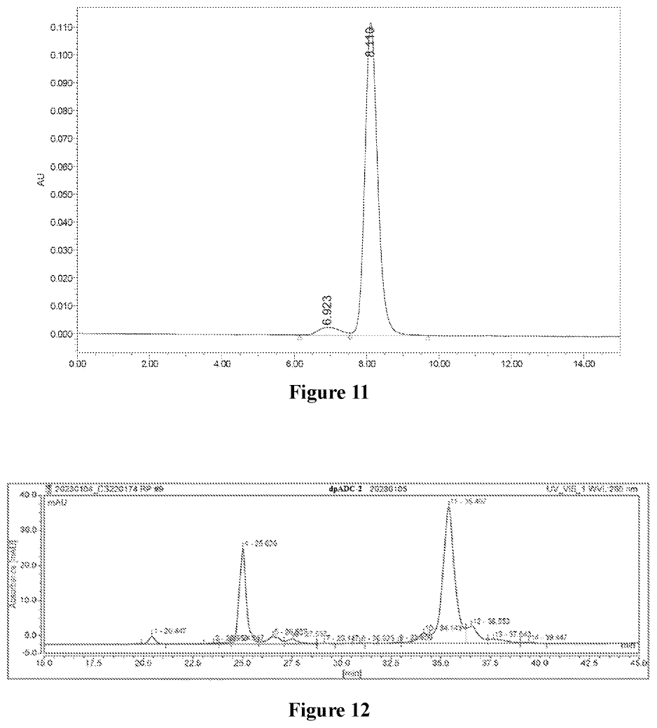

The results of the assay are shown in FIG. 11. The high molecular weight polymer in the ADC drug is <5%, and the ADC sample at 8.1 min is mainly in the form of monomer.

### 5.5 Preparation and Analysis of Antibody-Drug Conjugate dpADC-2 with Dual-Enzyme Catalyzed Site-Specific Conjugation

### 5.5.1 Preparation of the Antibody-Drug Conjugate dpADC-2

Preparation of dpADC-2 refers to the general preparation protocol described in Example 5.4. Compound 8 and 9 are used as the linker-payload for conjugation of dpADC-2.

### 5.5.2 Analysis of ADC Drug dpADC-2 by RP-HPLC

Testing method is similary to the general preparation protocol described in Example 5.4.

The results are as shown in FIG. 12, the sum of the relative peak areas of the unconjugated cytotoxic antibody heavy and light chains is <7%; the conjugated product is mainly a product with a DAR of 2+4, and the overall DAR value of ADC drugs is about 5.6 (LC+HC=1.87+3.73).

### 5.5.3 Analysis of ADC Drug dpADC-2 by SEC-HPLC

Testing method is similary to the general preparation protocol described in Example 5.4 of the present disclosure.

The detection results are as shown in FIG. 13, the high molecular weight polymer in the ADC drug <5%, and the ADC sample at 8.02 min is mainly in the form of monomer.

### 5.6 Preparation and Analysis of Antibody-Drug Conjugate dpADC-3 with Dual-Enzyme Catalyzed Site-Specific Conjugation

### 5.6.1 Preparation of the Antibody-Drug Conjugate dpADC-3

Preparation of dpADC-3 refers to the general preparation protocol described in Example 5.4. Compound 5 and 10 were used as the linker-payload for conjugation of dpADC-3, and the ErbB2/Her2 antibody was replaced to be ErbB3/Her3 antibody in the preparation.

### 5.6.2 Analysis of ADC Drug dpADC-3 by RP-HPLC

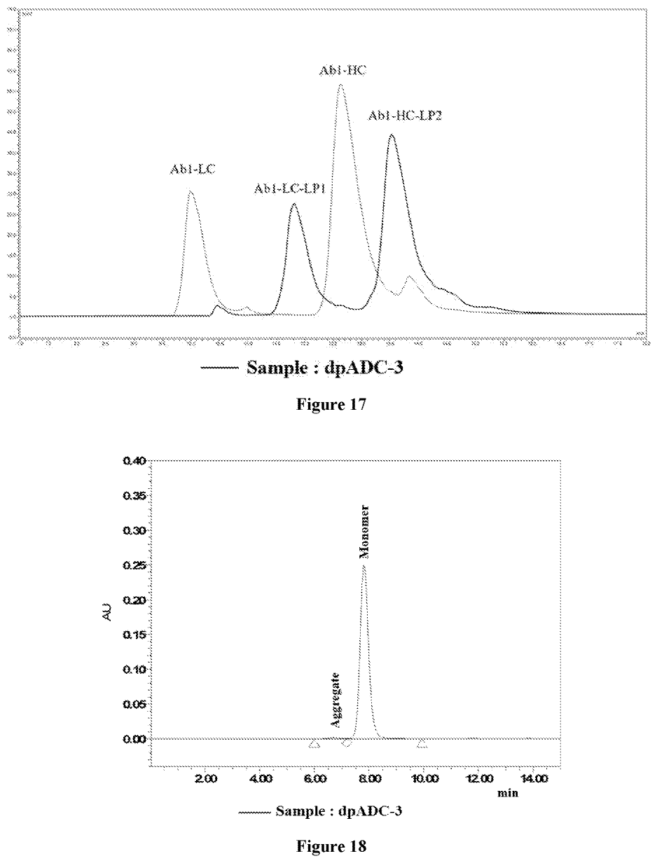

The detection results are shown as the following table and FIG. 17, the sum of the relative peak areas of the unconjugated cytotoxic antibody heavy and light chains is <8%; the majority of the conjugated product is product with a DAR of 2+4, and the overall DAR value of the dpADC drug is about 5.76 (LC+HC=1.93+3.83).

### The RP-HPLC Results of dpADC-3

| | | | | | | Conjunction |
|---|---|---|---|---|---|---|
| Sample | LC % | LC-LP1% | HC % | HC-LP2% | DAR value | efficiency % |
| dpADC-3 | 3.49 | 96.51 | 4.27 | 95.73 | 1.93 + 3.83 | 96.50 + 95.75 |

### 5.6.3 Analysis of ADC Drug dpADC-3 by SEC-HPLC

The results of the assay are shown in FIG. 18. The high molecular weight polymer in the ADC drug is <1%, and the dpADC sample at 7.81 min is mainly in the form of monomer.

### Example 6 the Effect of ADC Drug dpADC-1 on the Proliferation of Tumor Cells with Different Expression Levels of ErbB2/Her2

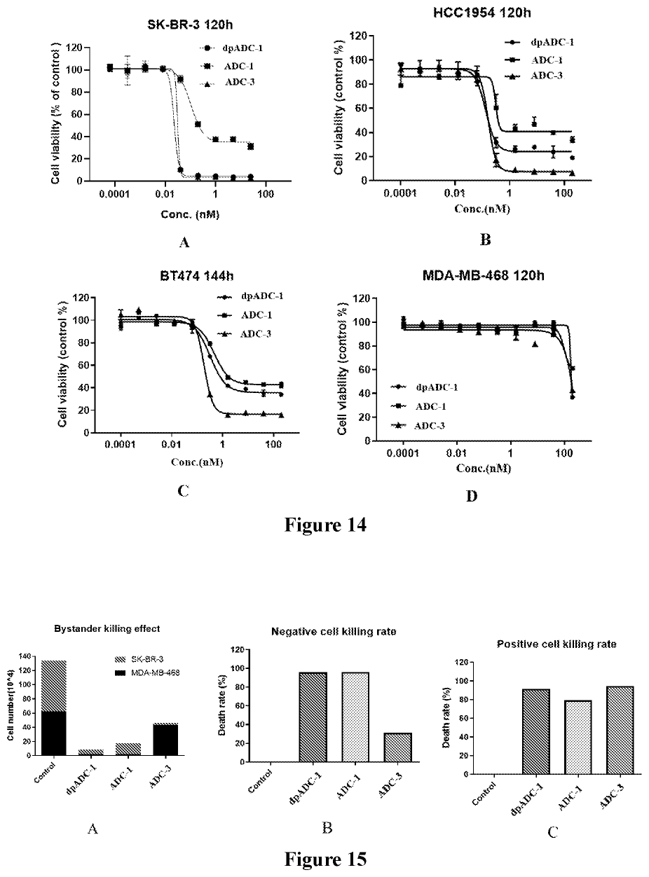

1) ErbB2/Her2 positive human breast cancer cells SK-BR-3, HCC1954, BT-474, and ErbB2/Her2 negative human breast cancer cells MDA-MB-468 were used to test the activity of the dpADC-1.
2) The results of the inhibitory effects of different drugs on the proliferation of tumor cells are shown in Table 3, Table 4 and FIG. 14A-D. The dpADC-1, ADC-3 and ADC-1 all have obvious inhibitory effects on the proliferation of ERB2/HER2 positive cells. The inhibitory effect of dp ACD-1 on the proliferation of ErbB2/Her2 positive cells is similar to or slightly weaker than ADC-3, but better than ADC-1. The dpADC-1 only shows a slight inhibitory effect on ErbB2/Her2 negative cells only at ≥200 nM revealing its good stability, which is consistent with ADC-3, and there is no decrease in stability caused by further conjugation with Compound 5.

TABLE 3
Inhibitory effects of different drugs on the
proliferation of tumor cells (IC₅₀, nM)

| | Drug | | |
|---|---|---|---|
| Cell line | dpADC-1 | ADC-3 | ADC-1 |
| SK-BR-3 | 0.02269 | 0.03115 | 0.1076 |
| HCC1954 | 0.1313 | 0.1630 | 0.4335 |
| BT-474 | 0.3226 | 0.1869 | 0.4643 |
| HepG2 | — | — | — |

TABLE 4
Proliferation inhibition of ErbB2/Her2-positive tumor
cells by different drugs (maximal killing, % control)

| | Drug | | |
|---|---|---|---|
| Cell line | dpADC-1 | ADC-3 | ADC-1 |
| SK-BR-3 | 95.81 | 96.80 | 65.17 |
| HCC1954 | 75.88 | 92.41 | 59.29 |
| BT-474 | 64.34 | 83.45 | 57.27 |

### Example 7 Bystander Killing Effect of ADC Drug dpADC-1 on ErbB2/Her2-Negative Tumor Cells

1) ErbB2/Her2 positive human breast cancer cells SK-BR-3 and ErbB2/Her2 negative human breast cancer cells MDA-MB-468 were used to test Bystander killing effect of the dpADC-1.
2) The proportion of ErbB2/Her2 positive and negative cell populations to the total cell population, respectively, was detected by Beckman flow cytometry. The number of viable cells in each well for both ErbB2/Her2 positive and negative cell populations was calculated and plotted using GraphPad software. The results are shown in FIG. 15A-C.

As shown in FIG. 15A, the overall cancer cell killing efficacy of dpADC-1 is significantly stronger than that of ADC-1 and ADC-3, indicating a synergistic effect of two payloads. As shown in FIG. 15B, in antigen-negative cancer cells (for example, ErbB2/Her2 negative human breast cancer cells MDA-MB-468), the killing effect of dpADC-1 is similar to that of ADC-1, and both are significantly stronger than ADC-3. As shown in FIG. 15C, in antigen-positive cancer cells (for example, ErbB2/Her2 positive human breast cancer cells SK-BR-3), the killing effect of dpADC-1 is slightly lower than ADC-3, but significantly stronger than ADC-1. In general, the conjugated drug dpADC-1 with two drugs has the strongest killing effect on cancer cells, which is stronger than that with a single drug.

### Example 8 the Effect of ADC Drug dpADC-2 on the Proliferation of Tumor Cells with Different Expression Levels of ErbB2/Her2

1) ErbB2/Her2 high expressing human breast cancer cells SK-BR-3, BT-474, medium expressing human breast cancer cells Jimt-1, low expressing human non-small cell lung cancer cells NCI-H2110, ErbB2/Her2 negative human hepatocellular carcinoma cells HepG2, were used for activity testing.
2) The results of the proliferation inhibitory effects of different drugs on tumor cells are shown in Table 5. The dpADC-2, ADC-2 and ADC-3 all showed significant proliferation inhibitory effects on ErbB2/HER2-positive cells. The dpADC-2 showed similar or slightly stronger proliferation inhibitory effects on ErbB2/HER2-positive cells than ADC-1, which was significantly better than ADC-3. The dpADC-2 shows no inhibitory effect on ErbB2/HER2-negative cells, and has good stability, which is consistent with that of ADC-3, with no decrease in stability due to additional conjugation with Compound 8.

TABLE 5
Proliferation inhibition of tumor cells by different drugs (IC₅₀, nM)

| | Drug | | |
|---|---|---|---|
| Cell line | dpADC-2 | ADC-3 | ADC-2 |
| SK-BR-3 | 0.009073 | 0.1006 | 0.01025 |
| BT-474 | 0.01112 | 0.3965 | 0.01321 |
| Jimt-1 | 0.04079 | — | 0.04848 |
| NCI-H2110 | 20-100 | 100-500 | 100-500 |
| HepG2 | — | — | — |

Note:
“—” presents not measured

### Example 9 Bystander Killing Effect of ADC Drug dpADC-2 on ErbB2/Her2-Negative Tumor Cells

1) ErbB2/HER2-positive human breast cancer cells SK-BR-3 and ErbB2/HER2-negative human breast cancer cells MDA-MB-468, in a ratio of SK-BR-3:MDA-MB-468=4:1 per well, were used to test bystander killing effect of the dpADC-2.
2) The proportion of ErbB2/HER2 positive and negative cell populations to the total cell population, respectively, was detected by Beckman flow cytometry. The number of viable cells in each well for both ErbB2/Her2 positive and negative cell populations was calculated and plotted using GraphPad software.

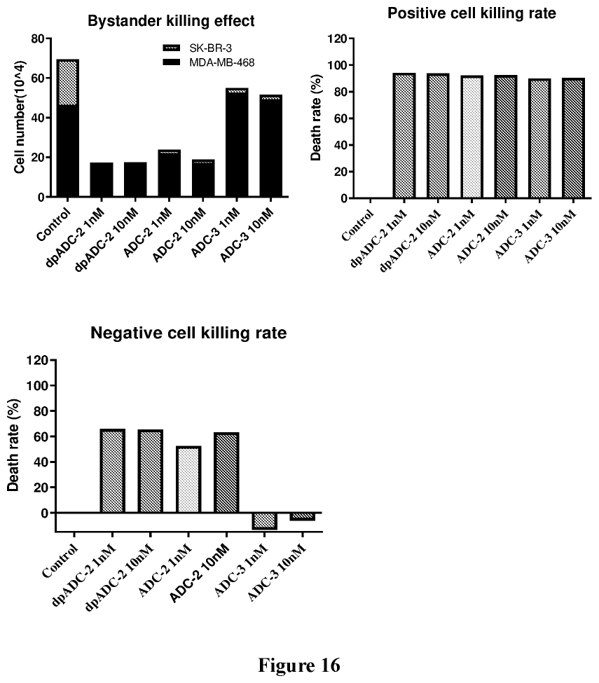

The results of the bystander killing effects of different drugs on tumor cells are as shown in FIG. 16. The dpADC-2 and ADC-2 both have significant bystander killing effects on ErbB2/HER2-negative cells, and dpADC-2 has slightly better bystander killing effects on ErbB2/HER2-negative cells than ADC-2 and significantly better than ADC-3, indicating a certain synergistic effect.

### Example 10 the Effect of ADC Drug dpADC-3 on the Osimertinib-Resistant NSCLC PDX Model Mice with Moderate Expression Levels of ErbB3/Her3 In Vivo

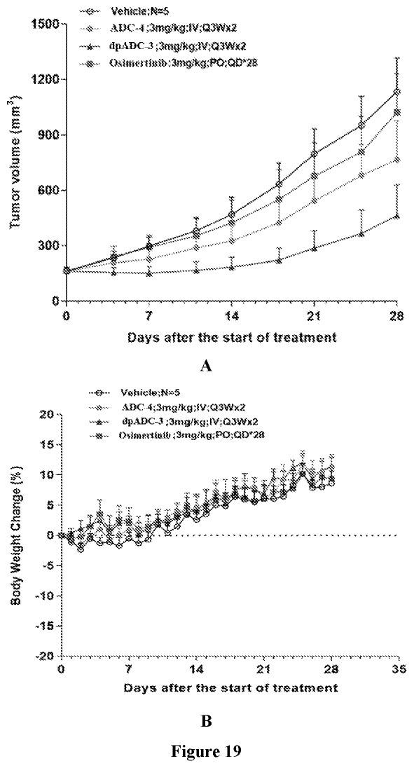

The tumor mass of transplanted human lung cancer in vivo was cut into tumor tissue with the size of about 3 mm×3 mm×3 mm (about 45-60 mg) and inoculated subcutaneously in NU/NU mice. The mice were administrated with vehicle, ADC-4, dpADC-3 and Osimertinib respectively when the average tumor volume was 163 mm³. ADC-4 is an endoglycosidase-catalyzed antibody-drug conjugete obtained by conjugating the ErbB3/Her3 antibody with the compound 5. The results are shown in FIG. 19A-B.

As shown in FIG. 19A, the inhibitory effect of dpADC-3 on tumor in mice is better than that of ADC-4 and Osimertinib alone. This shows that dpADC-3 has significant advantages over single-load ADC in the treatment of NSCLC resistant to Osimertinib. As shown in FIG. 19B, compared with the control group, there is no significant difference in the body weight change of mice in all drug treatment groups indicating that dpADC-3 has a good safety.

### Example 11 the Effect of ADC Drug dpADC-3 on the Osimertinib-Sensitive NSCLC PC-9 Model Mice with Low Expression Levels of ErbB3/Her3 In Vivo

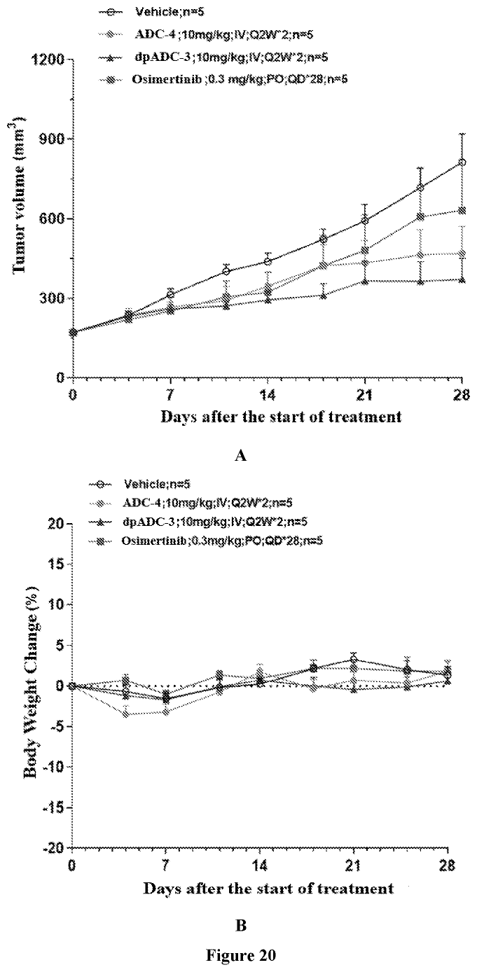

PC-9 tumor cells (10×10⁶/mouse) in 0.2 ml PBS (1:1) containing Matrigel were subcutaneously inoculated on the right side of each female BALB/c nude mouse for tumor development. The mice were administrated with vehicle, ADC-4, dpADC-3 and Osimertinib respectively when the average tumor volume was 172 mm³. The results are shown in FIG. 20A-B.

As shown in FIG. 20A, the inhibitory effect of dpADC-3 on tumor in mice is better than that of ADC-4 and Osimertinib alone. This shows that dpADC-3 has significant advantages over single-load ADC in the treatment of NSCLC sensitive to Osimertinib. As shown in FIG. 20B, compared with the control group, there is no significant difference in the body weight change of mice in all drug treatment groups indicating that dpADC-3 has a good safety.

The above embodiments are only intended to illustrate the technical concept and features of the disclosure and are intended to enable those skilled in the art to understand the contents of the disclosure and implement it, and do not limit the scope of protection of the disclosure. All equivalent variations or modifications made in accordance with the spirit and substance of the present disclosure shall be covered by the scope of protection of the present disclosure.

1. An enzymatic conjugation process for preparing an immunoligand/payload conjugate, comprising the following steps:
conjugating a first payload to an immunoligand through enzymatic catalysis of a first enzyme,
conjugating a second payload to the immunoligand through enzymatic catalysis of a second enzyme,
wherein the first enzyme is different from the second enzyme.

2. The process according to claim 1, wherein the enzymatic catalysis step of the first enzyme and the enzymatic catalysis step of the second enzyme are performed simultaneously or sequentially.

3. (canceled)

4. (canceled)

5. The process according to claim 1, wherein the first enzyme comprises a ligase and/or the second enzyme comprises an endoglycosidase, or wherein the first enzyme comprises an endoglycosidase and/or the second enzyme comprises a ligase.

6. (canceled)

7. The process according to claim 5, wherein the ligase is a transpeptidase or a variant thereof, such as sortase A, sortase B, sortase C, sortase D, sortase E or sortase F and a variant thereof, preferably sortase A;
and/or
the recognition motif of ligase donor substrate is LPXTGJ, NPXTG, LPXTA, or LAXTG; preferably, LPXTG or LPETGG;
and/or
the recognition motif of ligase receptor substrate is Gₙ, where G is glycine and n is an integer of 3-10;
X is any natural or unnatural amino acid;
J is absent, or is an amino acid fragment containing 1-10 amino acids, wherein each amino acid is independently any natural or unnatural amino acid; preferably, J is absent or is Gₘ, wherein m is an integer of 1-10.

8. The process according to claim 7, wherein the sortase A comprises an amino acid sequence selected from any one of SEQ ID NO:1-27 or an amino acid sequence having at least about 85%, at least about 90%, at least about 95% or at least about 99% sequence identity therewith;
preferably, the sortase A comprises an amino acid sequence of SEQ ID NO:27 or an amino acid sequence having at least about 85%, at least about 90%, at least about 95% or at least about 99% sequence identity therewith.

9. The process according to claim 5, wherein the endoglycosidase is N-acetylglucosaminidase, which covalently connects a donor containing oxazoline oligosaccharide to an immunoligand containing GlcNAc motif, and the donor containing oxazoline oligosaccharide further contains a payload.

10. The process according to claim 9, wherein the N-acetylglucosaminidase is at least one selected from Endo S (Streptococcus pyogenes endoglycosidase-S), Endo F3 (Elizabethkingia miricola endoglycosidase-F3), Endo S2 (Endoglycosidase S2, Streptococcus pyogenes endoglycosidase-S2), Endo Sd (Endoglycosidase-Sd, Streptococcus pyogenes endoglycosidase-Sd) and Endo CC (Endoglycosidase-CC, Streptococcus pyogenes endoglycosidase-CC) or a variant thereof;
the N-acetylglucosaminidase is at least one selected from Endo H, Endo D, Endo F2, Endo F3, Endo M, Endo CC1, Endo CC2, Endo Om, Endo S and Endo S2, or a variant thereof;
preferably, the N-acetylglucosaminidase is Endo S2 or a variant thereof.

11. The process according to claim 9, wherein the oxazoline oligosaccharide is one or more selected from the group consisting of: disaccharide oxazoline, trisaccharide oxazoline, tetrasaccharide oxazoline, pentasaccharide oxazoline, hexasaccharide oxazoline, heptasaccharide oxazoline, octasaccharide oxazoline, nonasaccharide oxazoline, decasaccharide oxazoline and undecasaccharide oxazoline.

12. The process according to claim 9, wherein the oxazoline oligosaccharide has the following structure:
the first hexose group or a derivative thereof-(the second hexose group or a derivative thereof)_{f}-β-D-glucopyranosyl oxazoline,
wherein f is 0, 1, 2, 3, 4, 5 or 6; β-D-glucopyranosyl oxazoline has the structure of:

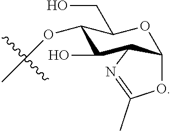

13. The process according to claim 12, wherein the first hexose group or a derivative thereof is selected from glucosyl, mannosyl, galactosyl, or a derivative thereof; and/or
the carbon at the 6-position of the first hexose group is in —C(O)— form; and/or
the second hexose group or a derivative thereof is independently selected from glucosyl, mannosyl, galactosyl or a derivative thereof in each occurrence; and/or
each monosaccharide portion of the oligosaccharide structure is linked by β-(1→4) glycosidic bonds; and/or
the first hexose group derivative and the second hexose group derivative are independently selected from a derivative in which a hydroxyl group of the uronic acid or monosaccharide is replaced by an acylamino group.

14. The process according to claim 12, wherein the oxazoline oligosaccharide has the following structure:
the first hexose group or a derivative thereof-β-D-glucopyranosyl oxazoline, wherein the first hexose group or a derivative thereof is mannose or a derivative thereof; or
the first hexose group or a derivative thereof-β-D-glucopyranosyl oxazoline, wherein the first hexose group or a derivative thereof is a galactose or a derivative thereof;
preferably, the structure of the oxazoline oligosaccharide is as follows:

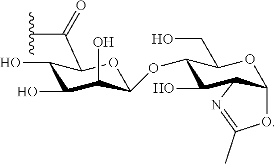

15. The process according to claim 5, wherein the ligase and/or endoglycosidase are covalently linked to a self-labelling protein tag to form a ligase fusion protein and/or endoglycosidase fusion protein, optionally wherein the self-labelling protein tag comprises a SNAP tag, a CLIP tag, a His tag, a Halo tag or a variant thereof.

16. (canceled)

17. The process according to claim 15, wherein in the ligase fusion protein, the ligase has an isoelectric point (pI) of about 7.5 to about 10.0, and the self-labelling protein tag is a Halo tag with an isoelectric point of about 4.5 to about 5.0, and the pI of the ligase fusion protein is about 2.0 to about 4.5 pH units lower than that of the ligase,
optionally wherein the Halo tag comprises the amino acid sequence of SEQ ID NO: 28 or an amino acid sequence having at least about 85%, at least about 90%, at least about 95% or at least about 99% sequence identity therewith.

18. (canceled)

19. The process according to claim 17, wherein the ligase of the ligase fusion protein is sortase A comprising an amino acid sequence selected from any one of SEQ ID NO: 1-27 or an amino acid sequence having at least about 85%, at least about 90%, at least about 95%, or at least about 99% sequence identity therewith;
preferably, the sortase A comprises an amino acid sequence of SEQ ID NO:27 or an amino acid sequence having at least about 85%, at least about 90%, at least about 95%, or at least about 99% sequence identity therewith,
optionally wherein the ligase fusion protein comprises the amino acid sequence of SEQ ID NO: 29 or an amino acid sequence having at least about 85%, at least about 90%, at least about 95%, or at least about 99% sequence identity therewith.

20. (canceled)

21. The process according to claim 15, wherein in the endoglycosidase fusion protein, the self-labelling protein tag is one or more selected from His tag and Halo tag,
optionally wherein
the amino terminus of endoglycosidase is covalently connected with a Halo tag; or
one end of endoglycosidase is covalently connected with a Halo tag, and the other end is covalently connected with a His tag; or
the amino terminus of endoglycosidase is covalently connected with a Halo tag, and the carboxyl terminus is covalently connected with a His tag; or
the amino terminus of endoglycosidase is covalently connected with a Halo tag, and the carboxyl terminus is covalently connected with a His tag, and the endoglycosidase is Endo S2.

22. (canceled)

23. The process according to claim 21, wherein the endoglycosidase fusion protein has an isoelectric point (pI) of about 4 to about 7, optionally wherein the endoglycosidase fusion protein comprises an amino acid sequence of SEQ ID NO: 30, or an amino acid sequence having at least 90% identity therewith, or an amino acid sequence having one or more conservative amino acid substitutions compared to SEQ ID NO: 30.

24. (canceled)

25. The process according to claim 1, wherein the first enzyme and the second enzyme are independently immobilized on a support, optionally wherein the first enzyme and the second enzyme are covalently immobilized on the support through a self-labelling protein tag, for example, a SNAP tag, a CLIP tag, a His tag, a Halo tag or a variant thereof;
preferably, the support comprises a haloalkyl linker, preferably a chloroalkyl linker, such that the first enzyme and the second enzyme are immobilized on the support by a covalent interaction between the haloalkyl linker and a Halo tag.

26. (canceled)

27. The process according to claim 25, wherein,
the support has the structure of formula (II): 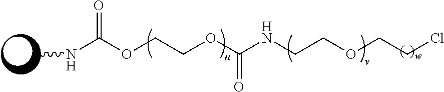
wherein u is an integer of 1 to 20, v is an integer of 0 to 20, and w is an integer of 1 to 19; [symbol]
is a resin, a bead, a membrane, a gel, a matrix, a film, a plate, a well, a tube, a slide or a surface, preferably a resin, more preferably an agarose resin, a silicone resin, a polymethyl methacrylate resin or a cellulose resin, most preferably a highly crosslinked agarose resin or a polymethyl methacrylate resin.

28. The process according to claim 1, wherein the immunoligand is selected from an antibody, a modified antibody format, an antibody derivative or fragment and/or a mimetic antibody; preferably, the immunoligand contains an Fc terminus, optionally wherein the antibody is selected from anti-CD19 antibody, anti-CD20 antibody, anti-CD22 antibody, anti-CD25 antibody, anti-CD30/TNFRSF8 antibody, anti-CD33 antibody, anti-CD37 antibody, anti-CD44v6 antibody, anti-CD56 antibody, anti-CD70 antibody, anti-CD71 antibody, anti-CD74 antibody, anti-CD79b antibody, anti-CD117/KITk antibody and anti-CD123 antibody, Anti-CD138 antibody, anti-CD142 antibody, anti-CD174 antibody, anti-CD227/MUC1 antibody, anti-CD352 antibody, anti-CLDN18.2 antibody, anti-DLL3 antibody, anti-ErbB2/HER2 antibody, anti-GPNMB antibody, anti-ENPP3 antibody, anti-Nectin-4 antibody, anti-EGFRvIII antibody, anti-SLC44A4/AGS-5 antibody, Anti-CEACAM5 antibody, anti-PSMA antibody, anti-TIM1 antibody, anti-LY6E antibody, anti-LIV1 antibody, anti-SLITRK6 antibody, anti-HGFR/cMet antibody, anti-SLAMF7/CS1 antibody, anti-EGFR antibody, anti-BCMA antibody, anti-AXL antibody, anti-NaPi2B antibody, anti-GCC antibody, anti-STEAP1 antibody, Anti-MUC16 antibody, anti-Mesothelin antibody, anti-ETBR antibody, anti-EphA2 antibody, anti-5T4 antibody, anti-FOLR1 antibody, anti-LAMP1 antibody, anti-Cadherin 6 antibody, anti-FGFR2 antibody, anti-FGFR3 antibody, anti-CA6 antibody, anti-CanAg antibody, anti-integrin aV antibody, anti-TDGF1 antibody, anti-Ephrin A4 antibody, anti-TROP2 antibody, anti-PTK7 antibody, anti-NOTCH3 antibody, anti-C4.4A antibody, anti-FLT3 antibody, anti-B7H_{3/4} antibody, anti-TF (Tissue Factor) antibody, anti-ROR1/2 antibody; preferably anti-CD19 antibody, anti-ErbB2/HER2 antibody, anti-CLDN18.2 antibody, anti-Nectin-4 antibody, anti-FGFR3 antibody and anti-Trop2 antibody; more preferably, anti-ErbB2/HER2 antibody.

29. (canceled)

30. The process according to claim 1, wherein the first payload and the second payload are independently selected from a small molecule compound (for example, a small molecule with various mechanisms of action, including various traditional small molecule drugs, a photoacoustic dynamic therapy drug, a photothermal therapy drug, etc., such as a chemotherapy drug, a small molecule targeted drug, an immune agonist, etc., for example, a traditional cytotoxic drug such as a cisplatin, paclitaxel, 5-fluorouracil, cyclophosphamide and bendamustine; a small molecule targeted drug, such as imatinib mesylate, gefitinib and anlotinib; an immune agonist, such as a STING agonist and a TLR agonist), a nucleic acid and a nucleic acid analogue, a tracer molecule (including a fluorescent molecule, a biotin, a fluorophore, a chromophore, a spin resonance probe and a radioactive label, etc.), a short peptide, a polypeptide, a peptidomimetic and a protein,
optionally wherein the first payload and the second payload are independently selected from a cytotoxin or a fragment thereof, preferably, the cytotoxin is selected from the group consisting of taxanes, maytansinoids, auristatins, epothilones, combretastatin A-4 phosphate, combretastatin A-4 and a derivative thereof, a indole-sulfonamides, vinblastines such as vinblastine, vincristine, vindesine, vinorelbine, vinflunine, vinglycinate, anhydrovinblastine, dolastatin 10 and an analogue thereof, halichondrin B, eribulin, indole-3-oxoacetamide, podophyllotoxin, 7-diethylamino-3-(2′-benzoxazolyl)-coumarin (DBC), discodermolide, laulimalide, camptothecins and a derivative thereof, mitoxantrone, mitoguanidine hydrazone, nitrogen mustard, nitrosoureasm, aziridines, benzodopa, carboquone, meturedepa, uredepa, alacinomycin, actinomycin, antramycin, bleomycins, actinomycin C, carabicin, carminomycin, sarkomycin, actinomycin D, daunorubicin, detorubicin, adriamycin, epirubicin, esorbicin, idarubicin, macellomycin, mitomycins, nogalamycin, olivomycin, peplomycin, porfiromycin, puromycin, ferric adriamycin, rodorubicin, rufocromomycin, streptozocin, zinostatins, zorubicin, trichothecene, T-2 toxin, verracurin A, bacillocporin A, anguidine, ubenimex, azaserine, 6-diazo-5-oxo-L-norleucine, dimethyl folic acid, methotrexate, pteropterin, trimetrexate, edatrexate, fludarabine, 6-mercaptopurine, thiamiprine, thioguanine, ancitabine, gemcitabine, enocitabine, azacitidine, 6-azauridine, carmofur, cytarabine, dideoxyuridine, doxifluridine, floxuridine, calusterone, dromostanolone propionate, epitiostanol, mepitiostane, testolactone, aminoglutethimide, mitotane, trilostane, flutamide, nilutamide, bicalutamide, leuprorelin acetate, protein kinase inhibitors and proteasome inhibitors; and/or
the cytotoxin is selected from vinblastines, colchicines, taxanes, auristatins, maytansinoids, calicheamicin, doxorubicin, duocarmycin, SN-38, cryptophycin analogue, deruxtecan, duocarmazine, centanamycin, dolastansine, pyrrolobenzodiazepine and exatecan and a derivative thereof; and/or
the cytotoxin is selected from auristatins, especially MMAE, MMAF or MMAD; and/or
the cytotoxin is selected from exatecan and a derivative thereof, for example DX8951f; and/or
the cytotoxin is selected from DXd-(1) and DXd-(2); preferably DXd-(1).

31. (canceled)

32. The process according to claim 1, wherein the immunoligand is an antibody, the payload is a drug, and the drug-antibody ratio (DAR) thereof is 2-16, preferably 4-12, more preferably 4-8, and most preferably 6-8, preferably the first-drug-antibody ratio is 2-4, the second-drug-antibody ratio is 2-4.

33. An immunoligand/payload conjugate, prepared by the process of claim 1, preferably an antibody-drug conjugate (ADC), more preferably a dual-drug antibody-drug conjugate (dpADC).

34. (canceled)

35. A method for treating a proliferative disease (for instance, cancer), comprising administering a therapeutically effective amount of the immunoligand/payload conjugate according to claim 33 to a subject in need thereof, optionally wherein the proliferative disease is bladder cancer, breast cancer, cervical cancer, colorectal cancer, endometrial carcinoma, gastric cancer, head and neck neoplasm, renal cancer, liver cancer, lung cancer, ovarian cancer, prostate cancer, esophageal cancer, gallbladder cancer, pancreatic cancer, thyroid cancer, skin cancer, leukemia, multiple myeloma, chronic lymphocytic lymphoma, adult T-cell leukemia, B-cell lymphoma, acute myeloid leukemia, Hodgkin lymphoma or non-Hodgkin lymphoma, Waldenstrom macroglobulinemia, hairy cell lymphoma, cell lymphoma, Burkitt lymphoma, glioblastoma, melanoma or rhabdomyosarcoma.

36. (canceled)


SEQUENCE LISTING (60 sequences)

SEQ ID NO: 1 - protein, 146 aa, synthetic construct
AKPQIPKDKSKVAGYIEIPDADIKEPVYPGPATPEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 2 - protein, 146 aa, synthetic construct
KTPEIPKDKSKMAGYIKVPDAEIEEPVYPGPATPEQLNRGVSFAEGNESLSDQNISIAGHTFTDRPHYQFTNLKAAKKGSKVYFKVGNETRKYKMTSIRDVDPSDVKVLDEHKGEKNQLTLITCDNYNKETGVWEKRKIFVAKEIK

SEQ ID NO: 3 - protein, 146 aa, synthetic construct
KAPAIPKDKSKMAGYIKVPDAEIEEPVYPGPATPEQLNRGVSFAEGNESLTDQNISIAGHTFTDRPHYQFTNLKAAKKGSKVYFKVGNETRKYKMTSIRNVDPSDVKVLDEHKGEKNQLTLITCDNYNKNTGVWEKRKIFVAKQIN

SEQ ID NO: 4 - protein, 146 aa, synthetic construct
KTPTIPKDKSKMAGYIEVPDAEIKEPVYPGPATLEQLNRGVSFAEGDESLDQQNISIAGHTFTDRPHYQFTNLKAAKKGSKVYFKVGNQTRKYKMTKIHDVNPSDVEVLDEQKGKKNQLTLITCDDYNEKTGVWEKRKIFIATQVN

SEQ ID NO: 5 - protein, 146 aa, synthetic construct
KAVEIPKDKSKMAGYIKIPDAEIEEPVYPGPATPEQLNRGVSFAEGNESLSDQNISIAGHTFTDRPHYQFTNLKAAKKGSKVYFKVGNETRKYKMTSIRDVDPSDVKVLDEHKGEKNQLTLITCDNYNKETGVWEKRKIFVAKEIK

SEQ ID NO: 6 - protein, 146 aa, synthetic construct
KAPEIPKDKSKMAGYIKVPDAEIEEPVYPGPATPEQLNRGVSFAEGNESLTDQNISIAGHTFTDRPHYQFTNLKAAKKGSKVYFKVGNETRKYKMTSIRDVDPSDVKVLDEHKGEKNQLTLITCDNYNKNTGVWEKRKIFVAKQIN

SEQ ID NO: 7 - protein, 146 aa, synthetic construct
KKPTIPKDKSKMAGYIEVPDAEIKEPVYPGPATPEQLNRGVSFAEGDESLDQQNIAIAGHTFTDRPHYQFTNLKAAKKGSKVYFKVGNEVRKYKMTKIHDVDPTEVKVLDEHKGKKNQLTLITCDDYNEQTGVWEKRKIFVATQVN

SEQ ID NO: 8 - protein, 146 aa, synthetic construct
KAVEIPKDKSKMVGYIKVPDAEIEEPVYPGPATPEQLNRGVSFAEGNESLSDQNISIAGHTFTDRPHYQFTNLKAAKKGSKVYFKVGNETRKYKMTSIRDVDPSDVKVLDEHKGEKNQLTLITCDNYNKETGVWEKRKIFVAKEIK

SEQ ID NO: 9 - protein, 146 aa, synthetic construct
ESPQIPKDKAKMAGYIEIPDAQIKEPVYPGPATPQQLNRGVSFAEGDESLNQQNISIAGHTFTDRPHYQFTNLKAAKKGSKVYFKVGNQTRKYKITKIHDVKPTEVKVLDEHPSKKNQLTLITCDDYNEQTGVWETRKIFVATQMN

SEQ ID NO: 10 - protein, 146 aa, synthetic construct
STPKIPSDKSKMAGYIEVPDAQIKEPVYPGPATPEQLNRGVSFAEGDESLNQQNISIAGHTFTDRPHYQFTNLKSAKIGSKVYFKTGNQTRKYKITKIRDVKPTEVKVLDEHPNKKNQLTLITCDDYNEETGVWETRKIFIATQIN

SEQ ID NO: 11 - protein, 146 aa, synthetic construct
ERPQIPKDKAKMAGYIEIPDAQIKEPVYPGPATPQQLNRGVSFAEGDESLYQQNISIAGHTFTDRPHYQFTNLKAAKKGSKVYFKVRNQTRKYKITKIHDVKPTEVKVLDEHPSKKNQLTLITCDDYNEQTGVWETRKIFVATQMN

SEQ ID NO: 12 - protein, 146 aa, synthetic construct
STPKIPSDKSKMAGYIEVPDAQIKEPVYPGPATPEQLNRGVSFAEGDESLNQQNISIAGHTFTDRSHYQFTNLKSAKIGSKVYFKTGNQTRKYKITKIRDVKPTEVKVLDEHPNKKNQLTLITCDDYNEETGVWETRKIFIATQIN

SEQ ID NO: 13 - protein, 146 aa, synthetic construct
EKPTISKDKSKMTGYISIPDADIKEPVYPGPATPEQLNRGVSFAEEDESLDDQNISIAGHTFIDRPNYQFTNLKAAKKGSKVTFKIGNETRKYKMTSIRDVNPEDVEVLDEHKGKKNQLTLITCDDYNENTGVWEKRKIFVAEEVK

SEQ ID NO: 14 - protein, 146 aa, synthetic construct
EKPTISKDKSKMTGYISIPDADIKEPVYPGPATPEQLNRGVSFAEEDESLDDQNISIAGHTFTDRPNYQFTNLKAAKKGSKVTFKIGNETRKYKMTSIRDVDPDAVEVLDENKGKKNQLTLITCDDYNENTGVWEKRKIFVAEQIK

SEQ ID NO: 15 - protein, 146 aa, synthetic construct
EKPTISKDKSKMTGYISIPDADIKEPVYPGPATPEQLNRGVSFAEEDESLDDQNISIAGHTFTDRPNYQFTNLKAAKKGSKVTFKTGNETRKYKMTSIRDVDPDAVEVLDENKGKKNQLTLITCDDYNENTGVWEKRKIFVAEQIK

SEQ ID NO: 16 - protein, 146 aa, synthetic construct
ETPTIPKDKSKMAGYISIPDAEIKEPVYPGPATPEQLNRGVSFAEEDEKLDDQNISIAGHTFIDRPHYQFTNLKAAKKGSKVYFKVGNETRKYKMTSIRDVNPDDVKVLDEHKGETNQLTLITCDNYNEQTGIWEKRKIFVAKQIN

SEQ ID NO: 17 - protein, 146 aa, synthetic construct
ETPTIPKDKSKMAGYISIPDAEIKEPVYPGPATPEQLDRGVSFAEEDEKLDDQNISIAGHTFIDRPHYQFTNLKAAKKGSKVYFKVGNETRKYKMTSIRDVNPDDVKVLDEHKGETNQLTLITCDNYNEQTGIWEKRKIFIAKQIN

SEQ ID NO: 18 - protein, 146 aa, synthetic construct
EKPTIPKDKSKMAGYISVPDAEIKEPVYPGPATPEQLNRGVSFAEGDESLDDQNISIAGHTFTDRPHYQFTNLKAAKKGSKVYFKVGDETREYKMTSIRDVDPEDVQVLDEHKGETNQLTLITCDNYNQQTGVWEKRKIFVAKQIK

SEQ ID NO: 19 - protein, 146 aa, synthetic construct
ERPTIPKNKSEMAGYISIPDAEIKEPVYPGPATLEQLNRGVSFAEGDESLDDQNISIAGHTFTDRPHYQFTNLKAAKKGSKVYFKVGDETREYKMTSIRDVDPEDVQVLDEHKGETNQLTLITCDNYNQQTGVWEKRKIFVAKQIK

SEQ ID NO: 20 - protein, 146 aa, synthetic construct
QTPTIPKDKSKMAGYISVPDAEIKEPVYPGPATPEQLNRGVSFAEEDESLSDQNISIAGHTFTDRPHYQFTNLKAAKKGSKVYFKIGNETREYKMTSIRDVNPDQVEVLNEHKGEKNQLTLITCDDYNEQTGVWEKRKIFVAKQVK

SEQ ID NO: 21 - protein, 146 aa, synthetic construct
QTPTIPKDKTKMAGYISVPDAEIKEPVYPGPATPEQLNRGVSFAEKDESLSDQNISIAGHTFTDRPHYQFTNLKAAKKGSKVYFKIGNETREYKMTSIRDVNPDEVEVLDEHKGEKNQLTLITCDDYNEQTGVWEKRKIFVAKQVK

SEQ ID NO: 22 - protein, 146 aa, synthetic construct
ERPTIPKDKSKMAGYISVPDAEIKEPVYPGPATLEQLNRGVSFAEGDESLDDQNISIAGHTFTDRPHYQFTNLKAAKKGSKVYFKVGDETREYKMTSIRDVNPEDVQVLDEHEGETNQLTLITCDNYNQQTGVWEKRKIFVAKQIK

SEQ ID NO: 23 - protein, 146 aa, synthetic construct
QTPTIPKDKTKMAGYISVPDAEIKEPVYPGPATPEQLNRGVSFAEKDESLSDQNISIAGHTFTDRPHYQFTNLKAAKKGSKVYFKIGNETREYKMTSIRDVNPDEVEVLDEHKGEKNQLTLITCDDYNEQTGVWEKRKIFVAKQVN

SEQ ID NO: 24 - protein, 146 aa, synthetic construct
DKPTIPKDKAEMAGYLRIPDADINEPVYPGPATPEQLNRGVSFAEEQESLDDQNIAIAGHTYIGRPHYQFTNLKAAKKGSKVYFKVGNETREYKMTTIRDVNPDEIDVLDEHRGDKNRLTLITCDDYNEKTGVWEKRKIFIAEQIK

SEQ ID NO: 25 - protein, 146 aa, synthetic construct
QTPTIPKDKTKMAGYISVPDAEIKEPVYPGPATPEQLNRGVSFTEKDESLSDQNISIAGHTFTDRPHYQFTNLKAAKKGSKVYFKIGNETREYKMTSIRDVNPDEVEVLDEHKGEKNQLTLITCDDYNEQTGVWEKRKIFVAKQVN

SEQ ID NO: 26 - protein, 146 aa, synthetic construct
DKPTIPKDKAEMAGYLRIPDADINEPVYPGPATPEQLNRGVSFAEEQESLDDQNIAIAGHTYIGRPHYQFTNLKAAKKGSKVYFKVGNETREYKMTTIRDVDPDEIDVLDEHRGDKNRLTLITCDDYNEKTGVWEKRKIFIAEQIK

SEQ ID NO: 27 - protein, 146 aa, synthetic construct
AKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTAVGVLDEQKGKDKQLTLITCDDYNEKTGVWETRKIFVATEVK

SEQ ID NO: 28 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 29 - protein, 453 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGSAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTAVGVLDEQKGKDKQLTLITCDDYNEKTGVWETRKIFVATEVK

SEQ ID NO: 30 - protein, 1160 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGSMDKHLLVKRTLGCVCAATLMGAALATHHDSLNTVKAEEKTVQTGKTDQQVGAKLVQEIREGKRGPLYAGYFRTWHDRASTGIDGKQQHPENTMAEVPKEVDILFVFHDHTASDSPFWSELKDSYVHKLHQQGTALVQTIGVNELNGRTGLSKDYPDTPEGNKALAAAIVKAFVTDRGVDGLDIDIEHEFTNKRTPEEDARALNVFKEIAQLIGKNGSDKSKLLIMDTTLSVENNPIFKGIAEDLDYLLRQYYGSQGGEAEVDTINSDWNQYQNYIDASQFMIGFSFFEESASKGNLWFDVNEYDPNNPEKGKDIEGTRAKKYAEWQPSTGGLKAGIFSYAIDRDGVAHVPSTYKNRTSTNLQRHEVDNISHTDYTVSRKLKTLMTEDKRYDVIDQKDIPDPALREQIIQQVGQYKGDLERYNKTLVLTGDKIQNLKGLEKLSKLQKLELRQLSNVKEITPELLPESMKKDAELVMVGMTGLEKLNLSGLNRQTLDGIDVNSITHLTSFDISHNSLDLSEKSEDRKLLMTLMEQVSNHQKITVKNTAFENQKPKGYYPQTYDTKEGHYDVDNAEHDILTDFVFGTVTKRNTFIGDEEAFAIYKEGAVDGRQYVSKDYTYEAFRKDYKGYKVHLTASNLGETVTSKVTATTDETYLVDVSDGEKVVHHMKLNIGSGAIMMENLAKGAKVIGTSGDFEQAKKIFDGEKSDRFFTWGQTNWIAFDLGEINLAKEWRLFNAETNTEIKTDSSLNVAKGRLQILKDTTIDLEKMDIKNRKEYLSNDENWTDVAQMDDAKAIFNSKLSNVLSRYWRFCVDGGASSYYPQYTELQILGQRLSNDVANTLKDHHHHHHHHHH

SEQ ID NO: 31 - protein, 222 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 32 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 228 aa, synthetic construct
DIEMTQSPDSLAVSLGERATINCRSSQSVLYSSSNRNYLAWYQQNPGQPPKLLIYWASTRESGVPDRFSGSGSGTDFTLTISSLQAEDVAVYYCQQYYSTPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGALPETGG

SEQ ID NO: 34 - protein, 447 aa, synthetic construct
QVQLQQWGAGLLKPSETLSLTCAVYGGSFSGYYWSWIRQPPGKGLEWIGEINHSGSTNYNPSLKSRVTISVETSKNQFSLKLSSVTAADTAVYYCARDKWTWYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 5 aa, synthetic construct
  VARIANT 3: note = X can be any single amino acid that is natural or unnatural
LPXTG

SEQ ID NO: 36 - protein, 4 aa, synthetic construct
GGGS

SEQ ID NO: 37 - protein, 10 aa, synthetic construct
GGGGSGGGGS

SEQ ID NO: 38 - protein, 5 aa, synthetic construct
  VARIANT 3: note = X can be any single amino acid that is natural or unnatural
NPXTG

SEQ ID NO: 39 - protein, 5 aa, synthetic construct
  VARIANT 3: note = X can be any single amino acid that is natural or unnatural
LPXTA

SEQ ID NO: 40 - protein, 5 aa, synthetic construct
  VARIANT 3: note = X can be any single amino acid that is natural or unnatural
LAXTG

SEQ ID NO: 41 - protein, 15 aa, synthetic construct
  VARIANT 3: note = X can be any single amino acid that is natural or unnatural
  VARIANT 6..15: note = up to 10 Xs could be deleted
LPXTGXXXXXXXXXX

SEQ ID NO: 42 - protein, 5 aa, synthetic construct
LPETG

SEQ ID NO: 43 - protein, 6 aa, synthetic construct
LPETGG

SEQ ID NO: 44 - protein, 5 aa, synthetic construct
NPQTN

SEQ ID NO: 45 - protein, 5 aa, synthetic construct
NPKTG

SEQ ID NO: 46 - protein, 7 aa, synthetic construct
LPQTSEQ

SEQ ID NO: 47 - protein, 14 aa, synthetic construct
  VARIANT 3: note = X can be any single amino acid that is natural or unnatural
  VARIANT 5..14: note = up to 7 Gs could be deleted
LPXTGGGGGGGGGG

SEQ ID NO: 48 - protein, 6 aa, synthetic construct
GLLQGA

SEQ ID NO: 49 - protein, 4 aa, synthetic construct
HHHH

SEQ ID NO: 50 - protein, 5 aa, synthetic construct
HHHHH

SEQ ID NO: 51 - protein, 6 aa, synthetic construct
HHHHHH

SEQ ID NO: 52 - protein, 10 aa, synthetic construct
HHHHHHHHHH

SEQ ID NO: 53 - protein, 4 aa, synthetic construct
SNAT

SEQ ID NO: 54 - protein, 4 aa, synthetic construct
YNAT

SEQ ID NO: 55 - protein, 4 aa, synthetic construct
WNDT

SEQ ID NO: 56 - protein, 4 aa, synthetic construct
VNNS

SEQ ID NO: 57 - protein, 5 aa, synthetic construct
  REGION >5: note = The entire sequence of amino acids 1-5 can be repeated 1-5 times.
GGGGS

SEQ ID NO: 58 - protein, 4 aa, synthetic construct
GFLG

SEQ ID NO: 59 - protein, 4 aa, synthetic construct
ALAL

SEQ ID NO: 60 - protein, 8 aa, synthetic construct
GALPETGG